SEQUENCE LISTING (90 sequences)

SEQ ID NO: 1 - protein, 82 aa, Homo sapiens
FPAMPLSSLFVNGPRTLCGAELVDALQFVCGDRGFYFNKPTGYGSSSRRAPQTGIVDECCFRSCDLRRLEMYCAPLKPAKSA

SEQ ID NO: 2 - protein, 22 aa, Unknown
  Description of Unknown PRELP heparin binding domain peptide
QPTRRPRPGTGPGRRPRPRPRP

SEQ ID NO: 3 - protein, 10 aa, Unknown
  Description of Unknown BMP-4 heparin binding domain peptide
RKKNPNCRRH

SEQ ID NO: 4 - protein, 8 aa, Unknown
  Description of Unknown Fibronectin heparin binding domain peptide
WQPPRARI

SEQ ID NO: 5 - protein, 21 aa, Unknown
  Description of Unknown Oncostatin M heparin binding domain peptide
LRKGVRRTRPSRKGKRLMTRG

SEQ ID NO: 6 - protein, 18 aa, Unknown
  Description of Unknown RAND1 heparin binding domain peptide
AVKRRPRFPAVKRRPRFP

SEQ ID NO: 7 - protein, 24 aa, Unknown
  Description of Unknown RAND2 heparin binding domain peptide
AKRRAARAAKRRAARAAKRRAARA

SEQ ID NO: 8 - protein, 13 aa, Unknown
  Description of Unknown Chondroadherin heparin binding domain peptide
KFPTKRSKKAGRH

SEQ ID NO: 9 - protein, 15 aa, Unknown
  Description of Unknown RAND3 heparin binding domain peptide
SKKARAGTGAKKARA

SEQ ID NO: 10 - protein, 19 aa, Unknown
  Description of Unknown RAND4 heparin binding domain peptide
ARKKAAKAGTGARKKAAKA

SEQ ID NO: 11 - protein, 19 aa, Unknown
  Description of Unknown Collagen IX heparin binding domain peptide
AVKRRPRFPVNSNSNGGNE

SEQ ID NO: 12 - protein, 18 aa, Unknown
  Description of Unknown RAND5 heparin binding domain peptide
AKKARAAKKARAAKKARA

SEQ ID NO: 13 - protein, 24 aa, Unknown
  Description of Unknown RAND6 heparin binding domain peptide
ARKKAAKAARKKAAKASRKKAAKA

SEQ ID NO: 14 - protein, 168 aa, Unknown
  Description of Unknown CNA-35 collagen binding domain polypeptide
ITSGNKSTNVTVHKSEAGTSSVFYYKTGDMLPEDTTHVRWFLNINNEKRYVSKDITIKDQIQGGQQLDLSTLNINVTGTHSNYYSGPNAITDFEKAFPGSKITVDNTKNTIDVTIPQGYGSLNSFSINYKTKITNEQQKEFVNNSQAWYQEHGKEEVNGKAFNHTVHN

SEQ ID NO: 15 - protein, 314 aa, Unknown
  Description of Unknown CNA-344 collagen binding domain polypeptide
RDISSTNVTDLTVSPSKIEDGGKTTVKMTFDDKNGKIQNGDTIKVAWPTSGTVKIEGYSKTVSLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKRYVSKDITIKDQIQGGQQLDLSTLNINVTGTHSNYYSGPNAITDFEKAFPGSKITVDNTKNTIDVTIPQGYGSLNSFSINYKTKITNEQQKEFVNNSQAWYQEHGKEEVNGKAFNHTVHNINANAGIEGTVKGELKVLKQDKDTKA

SEQ ID NO: 16 - protein, 81 aa, Unknown
  Description of Unknown Thrombospondin collagen binding domain polypeptide
KVSCPIMPCSNATVPDGECCPRCWPSDSADDGWSPWSEWTSCSTSCGNGIQQRGRSCDSLNNRCEGSSVQTRTCHIQECDK

SEQ ID NO: 17 - protein, 140 aa, Artificial Sequence
  Description of Artificial Sequence Synthetic polypeptide
MAWRLWWLLLLLLLLWPMVWAFPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSQPTRRPRPGTGPGRRPRPRPRP

SEQ ID NO: 18 - protein, 119 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSQPTRRPRPGTGPGRRPRPRPRP

SEQ ID NO: 19 - protein, 140 aa, Artificial Sequence
  Description of Artificial Sequence Synthetic polypeptide
MAWRLWWLLLLLLLLWPMVWAQPTRRPRPGTGPGRRPRPRPRPGGGGSGGGGSGGGGSFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 20 - protein, 119 aa, Artificial Sequence
  Description of Artificial Sequence Synthetic polypeptide
QPTRRPRPGTGPGRRPRPRPRPGGGGSGGGGSGGGGSFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 21 - protein, 228 aa, Unknown
  Description of Unknown Decorin collagen binding domain polypeptide
CPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKL

SEQ ID NO: 22 - protein, 233 aa, Unknown
  Description of Unknown Asporin collagen binding domain polypeptide
LFPMCPFGCQCYSRVVHCSDLGLTSVPTNIPFDTRMLDLQNNKIKEIKENDFKGLTSLYGLILNNNKLTKIHPKAFLTTKKLRRLYLSHNQLSEIPLNLPKSLAELRIHENKVKKIQKDTFKGMNALHVLEMSANPLDNNGIEPGAFEGVTVFHIRIAEAKLTSVPKGLPPTLLELHLDYNKISTVELEDFKRYKELQRLGLGNNKITDIENGSLANIPRVREIHLENNKLKK

SEQ ID NO: 23 - protein, 294 aa, Unknown
  Description of Unknown Chondroadherin collagen binding domain polypeptide
KLLNLQRNNFPVLAANSFRAMPNLVSLHLQHCQIREVAAGAFRGLKQLIYLYLSHNDIRVLRAGAFDDLTELTYLYLDHNKVTELPRGLLSPLVNLFILQLNNNKIRELRAGAFQGAKDLRWLYLSENALSSLQPGALDDVENLAKFHVDRNQLSSYPSAALSKLRVVEELKLSHNPLKSIPDNAFQSFGRYLETLWLDNTNLEKFSDGAFLGVTTLKHVHLENNRLNQLPSNFPFDSLETLALTNNPWKCTCQLRGLRRWLEAKASRPDATCASPAKFKGQHIRDTDAFRSCK

SEQ ID NO: 24 - protein, 350 aa, Unknown
  Description of Unknown Matrilin collagen binding domain polypeptide
RPLDLVFIIDSSRSVRPLEFTKVKTFVSRIIDTLDIGPADTRVAVVNYASTVKIEFQLQAYTDKQSLKQAVGRITPLSTGTMSGLAIQTAMDEAFTVEAGAREPSSNIPKVAIIVTDGRPQDQVNEVAARAQASGIELYAVGVDRADMASLKMMASEPLEEHVFYVETYGVIEKLSSRFQETFCALDPCVLGTHQCQHVCISDGEGKHHCECSQGYTLNADKKTCSALDRCALNTHGCEHICVNDRSGSYHCECYEGYTLNEDRKTCSAQDKCALGTHGCQHICVNDRTGSHHCECYEGYTLNADKKTCSVRDKCALGSHGCQHICVSDGAASYHCDCYPGYTLNEDKKT

SEQ ID NO: 25 - protein, 281 aa, Unknown
  Description of Unknown Fibromodulin collagen binding domain polypeptide
DCPQECDCPPNFLTAMYCDNRNLKYLPFVPSRMKYVYFQNNQITSIQEGVFDNATGLLWIALHGNQITSDKVGRKVFSKLRHLERLYLDHNNLTRMPGPLPRSLRELHLDHNQISRVPNNALEGLENLTALYLQHDEIQEVGSSMRGLRSLILLDLSYNHLRKVPDGLPSALEQLYMEHNNVYTVPDSYFRGAPKLLYVRLSHNSLTNNGLASNTFNSSSLLELDLSYNQLQKIPPVNTNLENLYLQGNRINEFSISSFCTVVDVVNFSKLQVVRLDGNEI

SEQ ID NO: 26 - protein, 291 aa, Unknown
  Description of Unknown PRELP collagen binding domain polypeptide
DCPRECYCPPDFPSALYCDSRNLRKVPVIPPRIHYLYLQSNFITELPVESFQNATGLRWINLDNNRIRKIDQRVLEKLPGLVFLYMEKNQLEEVPSALPRNLEQLRLSQNHISRIPPGVFSKLENLLLLDLQHNRLSDGVFKPDTFHGLKNLMQLNLAHNILRKMPPRVPTAIHQLYLDSNKIETIPNGYFKSFPNLAFIRLNYNKLTDRGLPKNSFNISNLLVLHLSHNRISSVPAINNRLEHLYLNNNSIEKINGTQICPNDLVAFHDFSSDLENVPHLRYLRLDGNYL

SEQ ID NO: 27 - protein, 716 aa, Unknown
  Description of Unknown COMP collagen binding domain polypeptide
DLGPQMLRELQETNAALQDVRELLRQQVREITFLKNTVMECDACGMQQSVRTGLPSVRPLLHCAPGFCFPGVACIQTESGARCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRRAQRFCPDGSPSECHEHADCVLERDGSRSCVCAVGWAGNGILCGRDTDLDGFPDEKLRCPERQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNGE

SEQ ID NO: 28 - protein, 116 aa, Artificial Sequence
  Description of Artificial Sequence Synthetic polypeptide
MAWRLWWLLLLLLLLWPMVWAFPAMPLSSLFVNGPRTLCGAELVDALQFVCGDRGFYFNKPTGYGSSSRRAPQTGIVDECCFRSCDLRRLEMYCAPLKPAKSAKFPTKRSKKAGRH

SEQ ID NO: 29 - protein, 95 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAKFPTKRSKKAGRH

SEQ ID NO: 30 - protein, 116 aa, Artificial Sequence
  Description of Artificial Sequence Synthetic polypeptide
MAWRLWWLLLLLLLLWPMVWAKFPTKRSKKAGRHFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 31 - protein, 95 aa, Artificial Sequence
  Description of Artificial Sequence Synthetic polypeptide
KFPTKRSKKAGRHFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 32 - protein, 92 aa, Artificial Sequence
  Description of Artificial Sequence Synthetic polypeptide
QPTRRPRPGTGPGRRPRPRPRPGPETLCGAXLVDALQFVCGDRGFYFNKPTGYGSSSRRAPQTGIVDXCCFRSCDLRRLEMYCAPLKPAKSA

SEQ ID NO: 33 - protein, 118 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASAVKRRPRFPVNSNSNGGNE

SEQ ID NO: 34 - protein, 267 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASITSGNKSTNVTVHKSEAGTSSVFYYKTGDMLPEDTTHVRWFLNINNEKRYVSKDITIKDQIQGGQQLDLSTLNINVTGTHSNYYSGPNAITDFEKAFPGSKITVDNTKNTIDVTIPQGYGSLNSFSINYKTKITNEQQKEFVNNSQAWYQEHGKEEVNGKAFNHTVHN

SEQ ID NO: 35 - protein, 413 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASRDISSTNVTDLTVSPSKIEDGGKTTVKMTFDDKNGKIQNGDTIKVAWPTSGTVKIEGYSKTVSLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKRYVSKDITIKDQIQGGQQLDLSTLNINVTGTHSNYYSGPNAITDFEKAFPGSKITVDNTKNTIDVTIPQGYGSLNSFSINYKTKITNEQQKEFVNNSQAWYQEHGKEEVNGKAFNHTVHNINANAGIEGTVKGELKVLKQDKDTKA

SEQ ID NO: 36 - protein, 109 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASRKKNPNCRRH

SEQ ID NO: 37 - protein, 107 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASWQPPRARI

SEQ ID NO: 38 - protein, 120 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASLRKGVRRTRPSRKGKRLMTRG

SEQ ID NO: 39 - protein, 115 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSAVKRRPRFPAVKRRPRFP

SEQ ID NO: 40 - protein, 121 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSAKRRAARAAKRRAARAAKRRAARA

SEQ ID NO: 41 - protein, 112 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASKFPTKRSKKAGRH

SEQ ID NO: 42 - protein, 112 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSSKKARAGTGAKKARA

SEQ ID NO: 43 - protein, 116 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSARKKAAKAGTGARKKAAKA

SEQ ID NO: 44 - protein, 115 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSAKKARAAKKARAAKKARA

SEQ ID NO: 45 - protein, 121 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSARKKAAKAARKKAAKASRKKAAKA

SEQ ID NO: 46 - protein, 180 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASKVSCPIMPCSNATVPDGECCPRCWPSDSADDGWSPWSEWTSCSTSCGNGIQQRGRSCDSLNNRCEGSSVQTRTCHIQECDK

SEQ ID NO: 47 - protein, 327 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKL

SEQ ID NO: 48 - protein, 332 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASLFPMCPFGCQCYSRVVHCSDLGLTSVPTNIPFDTRMLDLQNNKIKEIKENDFKGLTSLYGLILNNNKLTKIHPKAFLTTKKLRRLYLSHNQLSEIPLNLPKSLAELRIHENKVKKIQKDTFKGMNALHVLEMSANPLDNNGIEPGAFEGVTVFHIRIAEAKLTSVPKGLPPTLLELHLDYNKISTVELEDFKRYKELQRLGLGNNKITDIENGSLANIPRVREIHLENNKLKK

SEQ ID NO: 49 - protein, 393 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASKLLNLQRNNFPVLAANSFRAMPNLVSLHLQHCQIREVAAGAFRGLKQLIYLYLSHNDIRVLRAGAFDDLTELTYLYLDHNKVTELPRGLLSPLVNLFILQLNNNKIRELRAGAFQGAKDLRWLYLSENALSSLQPGALDDVENLAKFHVDRNQLSSYPSAALSKLRVVEELKLSHNPLKSIPDNAFQSFGRYLETLWLDNTNLEKFSDGAFLGVTTLKHVHLENNRLNQLPSNFPFDSLETLALTNNPWKCTCQLRGLRRWLEAKASRPDATCASPAKFKGQHIRDTDAFRSCK

SEQ ID NO: 50 - protein, 449 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASRPLDLVFIIDSSRSVRPLEFTKVKTFVSRIIDTLDIGPADTRVAVVNYASTVKIEFQLQAYTDKQSLKQAVGRITPLSTGTMSGLAIQTAMDEAFTVEAGAREPSSNIPKVAIIVTDGRPQDQVNEVAARAQASGIELYAVGVDRADMASLKMMASEPLEEHVFYVETYGVIEKLSSRFQETFCALDPCVLGTHQCQHVCISDGEGKHHCECSQGYTLNADKKTCSALDRCALNTHGCEHICVNDRSGSYHCECYEGYTLNEDRKTCSAQDKCALGTHGCQHICVNDRTGSHHCECYEGYTLNADKKTCSVRDKCALGSHGCQHICVSDGAASYHCDCYPGYTLNEDKKT

SEQ ID NO: 51 - protein, 380 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASDCPQECDCPPNFLTAMYCDNRNLKYLPFVPSRMKYVYFQNNQITSIQEGVFDNATGLLWIALHGNQITSDKVGRKVFSKLRHLERLYLDHNNLTRMPGPLPRSLRELHLDHNQISRVPNNALEGLENLTALYLQHDEIQEVGSSMRGLRSLILLDLSYNHLRKVPDGLPSALEQLYMEHNNVYTVPDSYFRGAPKLLYVRLSHNSLTNNGLASNTFNSSSLLELDLSYNQLQKIPPVNTNLENLYLQGNRINEFSISSFCTVVDVVNFSKLQVVRLDGNEI

SEQ ID NO: 52 - protein, 428 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSASDCPRECYCPPDFPSALYCDSRNLRKVPVIPPRIHYLYLDCPRECYCPPDFPSALYCDSRNLRKVPVIPPRIHYLYLQSNFITELPVESFQNATGLRWINLDNNRIRKIDQRVLEKLPGLVFLYMEKNQLEEVPSALPRNLEQLRLSQNHISRIPPGVFSKLENLLLLDLQHNRLSDGVFKPDTFHGLKNLMQLNLAHNILRKMPPRVPTAIHQLYLDSNKIETIPNGYFKSFPNLAFIRLNYNKLTDRGLPKNSFNISNLLVLHLSHNRISSVPAINNRLEHLYLNNNSIEKINGTQICPNDLVAFHDFSSDLENVPHLRYLRLDGNYL

SEQ ID NO: 53 - protein, 815 aa, Artificial Sequence [sequence omitted]

SEQ ID NO: 54 - protein, 15 aa, Unknown
  Description of Unknown AKK15 heparin binding domain peptide
AKKQRFRHRNRKGYR

SEQ ID NO: 55 - protein, 22 aa, Unknown
  Description of Unknown RLR22 heparin binding domain peptide
RLRAQSRQRSRPGRWHKVSVRW

SEQ ID NO: 56 - protein, 18 aa, Unknown
  Description of Unknown R1Q17 heparin binding domain peptide
RIQNLLKITNLRIKFVKL

SEQ ID NO: 57 - protein, 20 aa, Unknown
  Description of Unknown SEK20 heparin binding domain peptide
SEKTLRKWLKMFKKRQLELY

SEQ ID NO: 58 - protein, 24 aa, Unknown
  Description of Unknown ARK24 heparin binding domain peptide
ARKKAAKAARKKAAKAARKKAAKA

SEQ ID NO: 59 - protein, 24 aa, Unknown
  Description of Unknown AKK24 heparin binding domain peptide
AKKARAAKKARAAKKARAAKKARA

SEQ ID NO: 60 - protein, 28 aa, Unknown
  Description of Unknown AL1 heparin binding domain peptide
RPLREKMKPERRRPKGRGKRRREKQRPT

SEQ ID NO: 61 - protein, 21 aa, Unknown
  Description of Unknown AL2 heparin binding domain peptide
RRPKGRGKRRREKQRPTDAHL

SEQ ID NO: 62 - protein, 49 aa, Unknown
  Description of Unknown AL3 heparin binding domain polypeptide
QPTRRPRPGTGPGRRPRPRPRPTPSAPQPTRRPRPGTGPGRRPRPRPRP

SEQ ID NO: 63 - protein, 25 aa, Unknown
  Description of Unknown LGT25 heparin binding domain peptide
LGTRLRAQSRQRSRPGRWHKVSVRW

SEQ ID NO: 64 - protein, 12 aa, Unknown
  Description of Unknown Pep184 heparin binding domain peptide
SPWSEWTSSSTS

SEQ ID NO: 65 - protein, 12 aa, Unknown
  Description of Unknown Pep186 heparin binding domain peptide
GPWSPWDISSVT

SEQ ID NO: 66 - protein, 12 aa, Unknown
  Description of Unknown Pep185 heparin binding domain peptide
SHWSPWSSSSVT

SEQ ID NO: 67 - protein, 8 aa, Unknown
  Description of Unknown Pep239 heparin binding domain peptide
SHWSPWSS

SEQ ID NO: 68 - protein, 10 aa, Unknown
  Description of Unknown Pep246 heparin binding domain peptide
WSPWSSSSVT

SEQ ID NO: 69 - protein, 27 aa, Unknown
  Description of Unknown ATIII heparin binding domain peptide
AKLNSRLYRKANKSSKLVSANRLFGDK

SEQ ID NO: 70 - protein, 35 aa, Unknown
  Description of Unknown FibBeta heparin binding domain polypeptide
QGVNDNEEGFFSARGHRPLDKKREEAPSLRPAPPP

SEQ ID NO: 71 - protein, 112 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSAKKQRFRHRNRKGYR

SEQ ID NO: 72 - protein, 119 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSRLRAQSRQRSRPGRWHKVSVRW

SEQ ID NO: 73 - protein, 115 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSRIQNLLKITNLRIKFVKL

SEQ ID NO: 74 - protein, 117 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSSEKTLRKWLKMFKKRQLELY

SEQ ID NO: 75 - protein, 121 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSARKKAAKAARKKAAKAARKKAAKA

SEQ ID NO: 76 - protein, 121 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSAKKARAAKKARAAKKARAAKKARA

SEQ ID NO: 77 - protein, 125 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSRPLREKMKPERRRPKGRGKRRREKQRPT

SEQ ID NO: 78 - protein, 118 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSRRPKGRGKRRREKQRPTDAHL

SEQ ID NO: 79 - protein, 146 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSQPTRRPRPGTGPGRRPRPRPRPTPSAPQPTRRPRPGTGPGRRPRPRPRP

SEQ ID NO: 80 - protein, 122 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSLGTRLRAQSRQRSRPGRWHKVSVRW

SEQ ID NO: 81 - protein, 109 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSSPWSEWTSSSTS

SEQ ID NO: 82 - protein, 109 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSGPWSPWDISSVT

SEQ ID NO: 83 - protein, 109 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSSHWSPWSSSSVT

SEQ ID NO: 84 - protein, 105 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSSHWSPWSS

SEQ ID NO: 85 - protein, 107 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSWSPWSSSSVT

SEQ ID NO: 86 - protein, 124 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSAKLNSRLYRKANKSSKLVSANRLFGDK

SEQ ID NO: 87 - protein, 132 aa, Artificial Sequence
  Description of Artificial Sequence Synthetic polypeptide
FPAMPLSSLFVNGPRTLCGAELVDALQFVCGDRGFYFNKPTGYGSSSRRAPQTGIVDECCFRSCDLRRLEMYCAPLKPAKSAGGGGSGGGGSGGGGSQGVNDNEEGFFSARGHRPLDKKREEAPSLRPAPPP

SEQ ID NO: 88 - protein, 6 aa, Artificial Sequence
  Description of Artificial Sequence Synthetic 6xHis tag
HHHHHH

SEQ ID NO: 89 - protein, 11 aa, Artificial Sequence
  Description of Artificial Sequence Synthetic peptide
GGSGGHHHHHH

SEQ ID NO: 90 - protein, 15 aa, Artificial Sequence
  Description of Artificial Sequence Synthetic peptide
GGGGSGGGGSGGGGS

(19) United States
(12) Patent Application Publication
(10) Pub. No.: US 2016/0122411 A1
(43) Pub. Date: May 5, 2016

(54) CARTILAGE-BINDING FUSION PROTEINS

(71) Applicant: MERRIMACK PHARMACEUTICALS, INC., Cambridge, MA (US)

(72) Inventors: Emily Florine, Cambridge, MA (US); Dmitri B. Kirpotin, Revere, MA (US); Paul Kopesky, Lexington, MA (US); Alexey Lugovskoy, Woburn, MA (US); Rachel Rennard, Stoneham, MA (US); Birait Schoeberl, Cambridge, MA (US)

(73) Assignee: Merrimack Pharmaceuticals, Inc., Cambridge, MA (US)

(21) Appl. No.: 14/770,749

(22) PCT Filed: Mar. 28, 2014
(86) PCT No.: PCT/US14/32205

§ 371 Date: Aug. 26, 2015

Related U.S. Application Data
(60) Provisional application No. 61/806,599, filed on Mar. 29, 2013.

(51) Int. Cl. C07K 14/65; A61K 38/30; A61K 9/00; A61K 31/573; A61K 9/50; A61K 9/127; C07K 14/47; A61K 38/17

(52) U.S. Cl. CPC C07K 14/65; C07K 14/47; A61K 38/30; A61K 38/1709; A61K 31/573; A61K 9/5015; A61K 9/127; A61K 9/0019; C07K 2319/74

(57) ABSTRACT

Provided herein are fusion proteins comprising a first domain that specifically binds to the extracellular domain of a growth factor receptor, and a second domain that specifically binds to a cartilage matrix component, and pharmaceutical composition comprising these fusion proteins. Methods of treating musculoskeletal diseases using the fusion proteins and pharmaceutical composition disclosed herein are also provided.

### RELATED APPLICATIONS

This application claims priority to U.S. provisional application 61/806,599, filed Mar. 29, 2013, which is incorporated by reference herein in its entirety.

### BACKGROUND

Traumatic joint injury (e.g., tearing of ligaments, tendons, and cartilage) initiates a multi-factorial degenerative cascade within joint tissues that includes a chronic cycle of suppression of tissue repair, upregulation of extracellular matrix catabolism, cell death and joint degeneration. While some aspects of joint injury can be repaired by surgical tissue grafting procedures, these approaches can only partially restore the biomechanical stability of the joint. Current therapeutic approaches, including surgical and palliative therapies, are not sufficient to block permanent alteration of joint kinematics. Such alteration impacts the effects of physical forces and changes in cell or tissue mechanics (i.e., mechanobiological effects), including alteration of cell signaling, which contributes to joint pathophysiology. There are no existing pharmaceutical therapies that protect the joint tissues from the consequences of the altered cell signaling resulting from these mechanobiological effects. As a result, the risk of developing degenerative joint disease is dramatically increased in the years following a traumatic injury to a joint.

According to the CDC, in 2003, arthritis and other rheumatic conditions cost the United States $127.8 billion ($80.8 billion in medical care expenditures and $47.0 billion in lost earnings), or 1.2% of the Gross Domestic Product. Degenerative joint diseases resulting from traumatic joint injury account for more than 10% of the total burden of arthritis. Thus, there is a significant unmet need for effective treatments for degenerative joint disease resulting from traumatic joint injury. The following disclosure addresses this need and provides other benefits.

### SUMMARY

Delivering drugs directly to a diseased or damaged joint in a way that provides acceptable and effective therapy remains a significant challenge, as illustrated by the following statement in a recent publication:
“intra-articular therapy is challenging because of the rapid egress of injected materials from the joint space; this elimination is true of both small molecules, which exit via synovial capillaries, and of macromolecules, which are cleared by the lymphatic system. In general, soluble materials have an intra-articular dwell time measured only in hours.”
(Evans, et al., Nat. Rev. Rheumatol. 2014 January; 10(1):11-22)

That there has been a long felt and unmet need to meet this challenge is illustrated by the same problem being highlighted in another report published almost eight years earlier:
“A major improvement that should be targeted in future IA [intra-articular] treatment is a longer duration of action, since it is desirable to limit the number of IA injections per year due to the discomfort/pain associated with administration, as well as the possible risk of infection.”
(Gerwin, et al., Adv. Drug Deliv. Rev. 2006 May 20; 58(2):226-42)

Accordingly, provided herein are pharmaceutically active proteins that can be delivered directly to diseased or damaged so as to provide acceptable and effective therapy. These pharmaceutically active proteins are fusion proteins comprising a first domain that specifically binds to the extracellular domain of a growth factor receptor (e.g., IGF-1 receptor), and a second domain that specifically binds to a cartilage matrix component (e.g., sulfated glycosaminoglycan and collagen), and pharmaceutical composition comprising these fusion proteins. These fusion proteins and pharmaceutical compositions are particularly useful for treating degenerative joint diseases, such as osteoarthritis. Methods of treating musculoskeletal diseases using the fusion proteins and pharmaceutical composition disclosed herein are also provided.

In one aspect, the disclosure provides a fusion protein comprising a first binding domain and a second binding domain, wherein the first domain binds specifically to an extracellular domain of a growth factor receptor, and the second domain binds specifically to a cartilage matrix component, and within (i.e., when present in) the fusion protein, each binding domain exhibits specific binding activity.

In certain embodiments, the fusion protein is comprised of a single polypeptide chain. In certain embodiments, within (i.e., when present in) the fusion protein, each binding domain exhibits native binding activity.

In certain embodiments, the first domain is an IGF-1 receptor binding domain. In certain embodiments, the IGF-1 receptor binding domain has an amino acid sequence that comprises human IGF-1. In certain embodiments, the IGF-1 receptor binding domain has an amino acid sequence that comprises SEQ ID NO:1.

In certain embodiments, the second domain is an sGAG (sulfated glycosaminoglycan) binding domain. In certain embodiments, the sGAG binding domain has a sequence of, or a sequence homologous to, or substantially homologous to an sGAG binding domain of proline-arginine-rich end leucine-rich repeat protein (PRELP), chondroadherin (CHAD), oncostatin M, collagen IX, BMP-4, fibronectin, RAND1, RAND2, RAND3, RAND4, RAND5, RAND6, AKK15, RLR22, R1Q17, SEK20, ARK24, AKK24, AL1, AL2, AL3, LGT25, Pep184, Pep186, Pep185, Pep239, Pep246, ATIII, or FibBeta. In certain embodiments, the sGAG binding domain comprises an amino acid sequence selected from the group consisting of SEQ ID NO: 2-13, and 54-70 (see Table 1). In one particular embodiment, the sGAG binding domain comprises SEQ ID NO: 2. In one particular embodiment, the sGAG binding domain consists of SEQ ID NO: 2.

In certain embodiments, the second domain is a collagen binding domain. In certain embodiments, the collagen binding domain has a sequence of, or a sequence homologous to, or substantially homologous to the sequence of a collagen binding domain of matrilin, cartilage oligomeric matrix protein, PRELP, chondroadherin, fibromodulin, decorin, or asporin. In certain embodiments, the collagen binding domain comprises an amino acid sequence selected from the group consisting of SEQ ID NOs:14-16, and 21-27 (see Table 2).

In certain embodiments, the fusion protein comprises an amino acid sequence selected from SEQ ID NO: 17-20, 28-53, and 71-87 (see Table 3). In one particular embodiment, the fusion protein comprises the amino acid sequence set forth in SEQ ID NO:18. In one particular embodiment, the fusion protein consists of the amino acid sequence set forth in SEQ ID NO:18.

In certain embodiments, when present in the fusion protein, each binding domain exhibits native binding activity.

In certain embodiments, the fusion protein comprises fewer than 40,000, 35,000 30,000, 25,000, 20,000, 15,000, 10,000, 7,500, 5,000, 2,500, 1,000, 500, or 250 amino acids.

In certain embodiments, upon injection into an intra-articular space of a joint of a mammal, the fusion protein is retained within cartilage tissue of the joint for a period of time that is at least: 1.5 times, 2 times, 3 times, four times, five times, six times, seven times, eight times, nine times, ten times, twenty times, forty times, fifty times, sixty times, seventy times, eighty times, ninety times, or one hundred times longer than a fusion mutein which differs from the fusion protein only in that the second binding domain is a mutant domain that does not specifically bind to the cartilage matrix component. In certain embodiments, the joint is an injured joint or a diseased joint, and the amount of fusion protein retained in the cartilage tissue is at least about 5, about 10, about 20, or about 50 pmol/g of tissue. In certain embodiments, the mammal is a rat or a horse, the joint is an injured joint or a diseased joint, and 8, 9, 10, 11, 12, 13, or 14 days following the injection, the joint exhibits a reduction in loss of 1) sGAG from the cartilage tissue, 2) cell content, 3) total cartilage tissue, or 4) bone quality, when compared to loss of 1), 2), 3) or 4) of a matched control joint that has been injected with a control protein. In certain embodiments, the mammal is a rat or a horse, the joint is an injured joint or a diseased joint, and 8, 9, 10, 11, 12 13, or 14 days following the injection the cartilage tissue is characterized by an increase in production of sGAG in the cartilage tissue, when compared to production of sGAG in cartilage tissue of a matched control joint that has been injected with a control protein. In certain embodiments, the mammal is a rat or a horse, the joint is an injured joint or a diseased joint, and 8, 9, 10, 11, 12 13, or 14 days following the injection the cartilage tissue is characterized by an increase in levels of sGAG in the cartilage tissue, when compared to levels of sGAG in cartilage tissue of a matched control joint that has been injected with a control protein.

In certain embodiments, upon injection of the fusion protein into an intra-articular space of a joint of a mammal, the fusion protein is retained within cartilage tissue of the joint for a period of at least 8, at least 9, or at least 10 days. In certain embodiments, the joint is an injured joint, and the amount of fusion protein retained in the cartilage tissue is at least about 5, about 10, about 20, or about 50 pmol/g of tissue. In certain embodiments, the mammal is a rat or a horse, the joint is a diseased or injured joint, and 8, 9, 10, 11, 12, 13, or 14 days following the injection, the joint exhibits a reduction in loss of sGAG from the cartilage tissue, when compared to loss of sGAG in cartilage tissue of a matched control joint that has been injected with a control protein. In certain embodiments, the mammal is a rat or a horse, the joint is an injured joint, and 8, 9, 10, 11, 12 13, or 14 days following the injection the cartilage tissue is characterized by an increase in production of sGAG in the cartilage tissue, when compared to production of sGAG in cartilage tissue of a matched control joint that has been injected with a control protein. In certain embodiments, the mammal is a rat or a horse, the joint is an injured joint, and 8, 9, 10, 11, 12 13, or 14 days following the injection the cartilage tissue is characterized by an increase in levels of sGAG in the cartilage tissue, when compared to levels of sGAG in cartilage tissue of a matched control joint that has been injected with a control protein.

In another aspect, the disclosure provides a composition comprising one or more of the fusion proteins disclosed herein and a glucocorticoid. Suitable glucocorticoids include, without limitation, alclometasone, beclometasone, betamethasone, budesonide, chloroprednisone, ciclesonide, cortisol, cortisporin, cortivazol, deflazacort, dexamethasone, fludroxycortide, flunisolide, fluocinonide, fluocortolone, fluorometholone, fluticasone, hexacetonhydrocortamate, hydrocortisone, meprednisone, methylprednisolone, mometasone, paramethasone, prednisolone, prednisone, prednylidene, pregnadiene, pregnatriene, pregnene, proctosedyl, rimexolone, tetrahydrocorticosterone, triamcinolone and ulobetasol, and pharmaceutically acceptable salts, hydrates and esters thereof. In certain embodiments, the glucocorticoid is present at a concentration of 1-1000 μg/g of the composition. In certain embodiments, the in the glucocorticoid is conjugated to a fatty acid and the conjugation to the fatty acid is optionally via an ester bond. In certain embodiments, the fatty acid comprises palmitic acid.

In certain embodiments, the glucocorticoid is contained in a microparticle carrier. In certain embodiments, the microparticle carrier is a liposome. In certain embodiments, the microparticle carrier is a multilamellar vesicle. In certain embodiments, the microparticle carrier comprises a high melting temperature lipid. In certain embodiments, the lipid comprises distearoylphosphatidylcholine (DSPC), Dipalmitoylphosphatidylcholine (DPPC) or Hydro Soy phosphatidylcholine (HSPC). In certain embodiments, the glucocorticoid is present in the microparticle carrier at a concentration of between 0.1-20 molar percent of the microparticle carrier lipid.

In certain embodiments, the invention provided a composition comprising a fusion protein having the amino acid sequence set forth in SEQ ID NO:18 and dexamethasone 21-palmitate, wherein the dexamethasone21-palmitate is contained in a HSPC-containing multilamellar vesicle.

In certain embodiments, after injection of a fusion protein/glucocorticoid composition disclosed herein into an intra-articular space of an injured joint or a diseased joint, cartilage matrix synthesis readouts or cartilage degradation readouts are obtained, and the readouts show improvement over control readouts obtained after matched injection of a matched composition without glucocorticoid.

In another aspect, the disclosure provides a method of treatment of a joint injury or disease, the method comprising administration into an intra-articular space of a joint, a therapeutically effective amount of a fusion protein or composition disclosed herein. In certain embodiments, the joint injury or disease is selected from osteoarthritis, rheumatoid arthritis, cartilage degradation, acute inflammatory arthritis, infectious arthritis, osteoporosis, a drug toxicity-related cartilage defect, or a traumatic cartilage injury.

In another aspect, the disclosure provides a composition comprising one or more of the fusion proteins disclosed herein in a biocompatible hydrogel.

In certain embodiments, the hydrogel comprises one or more of hyaluronic acid (HA), an HA derivative, a cellulose derivative, and a heparin-like domain polymer.

In certain embodiments, the hydrogel comprises methylcellulose. Any molecular weight of methylcellulose can be employed, e.g., between about 5 kDa and about 500 kDa. Any amount of methylcellulose can be employed in the hydrogels. In certain embodiments, the amount of methylcellulose is between about 1 and about 10% by weight of the hydrogel.

In certain embodiments, the hydrogel comprises HA (e.g., sodium hyaluronate). Any molecular weight of HA can be employed, e.g., between about 10 kDa to about 1.8 MDa. Any amount of HA can be employed in the hydrogels. In certain embodiments, the amount of HA is between about 1 and about 10% by weight of the hydrogel.

In certain embodiments, the hydrogel comprises a heparin-like domain polymer that comprises chondroitin sulfate, heparan sulfate, or heparin. Any amount of heparin-like domain polymer can be employed in the hydrogels. In certain embodiments, the amount of heparin-like domain polymer is between about 0.05% and 2% by weight of the hydrogel.

In certain embodiments, the hydrogel is thermo-setting above a certain temperature (e.g., above 35° C.). In certain embodiments, the hydrogel is fluid or shear-thinning below a certain temperature (e.g., below 35° C.).

In certain embodiments, the fusion protein is present at a concentration of between about 1 and about 1000 μg/g of a hydrogel disclosed herein. In certain embodiments, the fusion protein is present at a concentration of between about 100 and about 10,000 μg/g of a hydrogel disclosed herein.

In certain embodiments, the hydrogel further comprise a glucocorticoid. Suitable glucocorticoids include, without limitation, alclometasone, beclometasone, betamethasone, budesonide, chloroprednisone, ciclesonide, cortisol, cortisporin, cortivazol, deflazacort, dexamethasone, fludroxycortide, flunisolide, fluocinonide, fluocortolone, fluorometholone, fluticasone, hexacetonhydrocortamate, hydrocortisone, meprednisone, methylprednisolone, mometasone, paramethasone, prednisolone, prednisone, prednylidene, pregnadiene, pregnatriene, pregnene, proctosedyl, rimexolone, tetrahydrocorticosterone, triamcinolone and ulobetasol, and pharmaceutically acceptable salts, hydrates and esters thereof. Modified glucocorticoids can also be employed. In certain embodiments, the glucocorticoid is conjugated to a fatty acid (e.g., palmitic acid) via an ester bond. In certain embodiments, the glucocorticoid is contained in a microparticle carrier, such as a liposome or multilamellar vesicle. Liposomal microparticle can comprise a high melting temperature (Tₘ) lipid e.g., DSPC (distearoyl phosphatidylcholine), DPPC (dipalmitoyl phosphatidylcholine) or HSPC (hydrogenated soy phosphatidylcholine). In certain embodiments, the glucocorticoid is contained in a liposomal microparticle and is present at between 0.1-20 molar percent of the liposome lipid. In certain embodiments, glucocorticoid is contained in a liposomal microparticle and the liposome lipid is between 0.01%-10% by weight of the hydrogel. In certain embodiments, the glucocorticoid is present in the hydrogel at a concentration sufficient to stimulate cartilage matrix synthesis or stimulate cell survival or prevent cartilage matrix degradation or prevent cell death when the pharmaceutical composition (e.g., a hydrogel) is injected into a joint. In certain embodiments, the glucocorticoid is present at a concentration between 1-1000 μg/g of hydrogel.

In certain embodiments, after injection of the composition into an intra-articular space of a joint, the cartilage matrix synthesis or degradation readouts of the joint show improvement over the readouts after injection of the fusion protein or the combination of the fusion protein plus glucocorticoid alone.

In certain embodiments, after injection of the composition into an intra-articular space of a joint, the glucocorticoid is present in the joint with a half-life of at least about 8 days (e.g., 9, 10, 11, or 12 days).

In certain embodiments, after injection of the composition into an intra-articular space of a joint, the fusion protein is retained in the intra-articular space of the joint for a longer time than either the fusion protein or glucocorticoid when injected alone.

In another aspect, the disclosure provides a method of treatment of a musculoskeletal disease, comprising the administration into an intra-articular space of a joint a therapeutically effective amount of one or more of the fusion proteins disclosed herein. In certain embodiments, the musculoskeletal disease comprises osteoarthritis, rheumatoid arthritis, post-injury cartilage degradation, acute inflammatory arthritis, infectious arthritis, osteoporosis, or is a result of drug toxicity.

### BRIEF DESCRIPTION OF THE DRAWINGS

FIG. 1 depicts graphs of fusion proteins disclosed herein binding to (A) heparan sulfate, and (B) chondroitin sulfate showing binding of GF-Fus3 (SEQ ID:18), but not GF-Fus1 (SEQ ID:1) or GF-Fus4 (SEQ ID:33) to heparan and chondroitin sulfate.

FIG. 2 depicts a graph of fusion proteins disclosed herein binding to collagen showing greater binding of GF-Fus5 (SEQ ID:34) than GF-Fus6 (SEQ ID:35) to collagen.

FIG. 3 depicts two graphs showing stimulation of AKT phosphorylation in bovine chondrocytes (A) and BXPC-3 cells (B) by GF-Fus1, GF-Fus2 (SEQ ID:32), GF-Fus3, GF-Fus4, GF-Fus5, GF-Fus6 fusion proteins and wild-type IGF, showing that all fusion proteins upregulated pAKT to a level comparable to upregulation by wild-type IGF. Data are mean±SEM.

FIG. 4 depicts a graph of sGAG loss against time (days) showing sGAG loss from bovine cartilage explants is reduced by GF-Fus1, GF-Fus2, and GF-Fus3 and by wild-type IGF. Data are mean±SEM.

FIG. 5 depicts two graphs of ³⁵S-sulfate incorporation into bovine cartilage explants showing an increase vs Disease control obtained by continuously adding GF-Fus1, GF-Fus2, GF-Fus3 and wild-type IGF (black bars). 4 or 8 days after removal from the culture medium (white bars), GF-Fus3 stimulated the largest increase in proteoglycan biosynthesis. ³⁵S-sulfate incorporation was measured during the final 48 hours of cultures ending on day 8 (FIG. 5A) and day 12 (FIG. 5B). Data are mean±SEM. No treatment control (Healthy) ³⁵S-sulfate incorporation rates were 132.2±3.6 and 140.3±11.0 (mean±SEM) pmol/hr/μg DNA at day 8 and day 12, respectively.

FIG. 6. is a graph of % sGAG loss against time (days) showing that % sGAG loss from bovine cartilage explants is reduced by GF-Fus1, GF-Fus3, GF-Fus5, and GF-Fus6 when these fusion proteins are supplied in every medium change. Data are mean±SEM.

FIG. 7. is a graph of % sGAG loss against time (days) showing a greater reduction in % sGAG loss from bovine cartilage explants for GF-Fus3 than for GF-Fus1 when added to the medium for day 0-4 only. Data are mean±SEM.

FIG. 8. presents two graphs of ³⁵S-sulfate incorporation into bovine cartilage explants showing an increase of such incorporation vs. Disease control by GF-Fus1, GF-Fus3, GF-Fus5, and GF-Fus6 when added in every medium change. GF-Fus3 stimulated the largest increase in ³⁵S-sulfate incorporation when added from day 0-4 only. ³⁵S-sulfate incorporation was measured during the final 48 hours of cultures ending on day 8 (FIG. 8A) and day 12 (FIG. 8B). Data are mean±SEM. No treatment control (Healthy)³⁵S-sulfate incorporation rates were 0.117±0.0099 and 0.083±0.0047 (mean±SEM) nmol/hr/μg DNA at day 8 and day 12, respectively.

FIG. 9: (A) is a graph of % sGAG loss against time (days) and (B) is a graph of ³⁵S-sulfate incorporation against time (days) for bovine explants treated with GF-Fus3 and Anti-Infl-1 (dexamethasone) singly and in combination. The largest reduction in % sGAG loss and increase in ³⁵S-sulfate incorporation vs. disease control was obtained with the combination of GF-Fus3 with Anti-Infl-2 (dexamethasone-21-palmitate). ³⁵S-sulfate incorporation was measured during the final 48 hours of cultures ending on day 8 and 12 (FIG. 9B). Data are mean±SEM. No treatment control (Healthy)³⁵S-sulfate incorporation rates were 153.5±9.1 and 123.2±8.8 (mean±SEM) pmol/hr/μg DNA at day 8 and day 12, respectively.

FIG. 10 presents graphs of: (A) cumulative release of GF-Fus2 from Gel 4; (B) cumulative release of GF-Fus2 from Gel 3; (C) per time point release of GF-Fus2 from Gel 4; (D) per time point release of GF-Fus2 from Gel 3; (E) cumulative release of wild type IGF from Gel 4; (F) cumulative release of wild type IGF from Gel 3; (G) per time point release of wild type IGF from Gel 4; (H) per time point release of wild type IGF from Gel 3. GF-Fus2 and wild type IGF were released from both Gel 3 and Gel 4 at similar rates from day 0-3 with no further release after day 4. Data are mean±SEM.

FIGS. 11 A, B, and C are graphs of cumulative release of Anti-Infl-2 (dexamethasone-21-palmitate) against time (days) from hydrogel formulations disclosed herein using the naming convention GelX-Y, where X is 1 or 2 for Gel 1 and 2, respectively, and Y is 1-5 to indicate nanoparticle type. The release rate of Anti-Infl-2 was varied to achieve a 4-fold difference in cumulative release at day 9 between the fastest (Gel2-3) and slowest (Gels1-1 and 1-3) releasing formulations. Data are mean±SEM.

FIG. 12 presents graphs of the % sGAG loss from (A) human ankle dome of talus cartilage explants; (B) human ankle posterior talus cartilage explants; (C) human ankle cartilage explants pooled from the head of the talus and the tibial and fibular malleolus; and (D) human knee femoral-patellar groove cartilage explants. Explants were treated with GF-Fus3 and Anti-Infl-1 (dexamethethasone) singly and in combination during 16 days (16 D) of culture with cytokines (Disease). No cytokine control (Healthy). Anti-Infl-1 reduced % sGAG loss for all tissues both singly and in combination with GF-Fus3. Data are mean±SEM.

FIG. 13 presents graphs of sulfated matrix biosynthesis as determined by ³⁵S-sulfate incorporation for treatments with GF-Fus3 and Anti-Infl-1 (dexamethasone) for 16 days (16 D) both singly and in combination. Incorporation was measured during the final 48 hours of a 16 day culture with cytokines (Disease) for (A) human ankle dome of talus cartilage explants; (B) human ankle posterior talus cartilage explants; (C) human ankle cartilage explants pooled from the head of the talus and the tibial and fibular malleolus; (D) human knee femoral-patellar groove cartilage explants; and (E) human knee chondyle cartilage explants. GF-Fus3 increased matrix biosynthesis vs. Disease control both singly and in combination with Anti-Infl-1 (dexamethasone). Healthy control incorporation rates were 89.3±13.0, 83.1±9.8, 72.6±8.8, 88.8±13.8, and 82.2±9.9 pmol/hr/μg DNA for the tissues in 13A-E, respectively. Data are mean±SEM.

FIG. 14 presents graphs of sulfated matrix biosynthesis for 8 and 16 day treatments (8 D and 16 D, respectively) with each of GF-Fus1 and GF-Fus3 in combination with Anti-Infl-1 (dexamethasone) for 16 days in the presence of cytokines (Disease) as determined by ³⁵S-sulfate incorporation for (A) human ankle dome of talus cartilage, (B) human ankle posterior talus cartilage, (C) human ankle head of talus and tibial and fibular malleolus cartilage, and (D) human knee femoral-patellar groove cartilage. ³⁵S-sulfate incorporation was measured during the final 48 hours of a 16 day culture with cytokines (Disease). Healthy control incorporation rates were 89.3±13.0, 83.1±9.8, 72.6±8.8, and 88.8±13.8 pmol/hr/μg DNA for the tissues in 14A-D, respectively. 16 day treatments with each of GF-Fus1 and GF-Fus3 (black bars) stimulated ³⁵S-sulfate incorporation vs. Disease control, but only GF-Fus3 stimulated ³⁵S-sulfate incorporation with 8 days of treatment (white bars) (E) Sulfated matrix biosynthesis for 8 and 16 day treatments with each of GF-Fus1 and GF-Fus3 in combination with Anti-Infl-1 for 16 days in the absence of cytokines as determined by ³⁵S-sulfate incorporation. Human knee chondyle cartilage explant incorporation was measured during final 48 hours of a 16 day culture. No cytokine (Healthy), cytokine (Disease) and Anti-Infl-1 alone included as controls. 16 day treatments with each of GF-Fus1 and GF-Fus3 (black bars) stimulated ³⁵S-sulfate incorporation vs. Disease control and vs. Anti-Infl-1 alone, but only GF-Fus3 stimulated ³⁵S-sulfate incorporation with 8 days of treatment (white bars). Data are mean±SEM.

FIG. 15 is a series of graphs depicting: (A) dexamethasone concentration in cartilage lysates; (B) dexamethasone concentration in meniscus lysates. (C) dexamethasone concentration in ligament lysates; D) dexamethasone concentration in patella plus surrounding synovium lysates; (E) dexamethasone concentration in serum; (F) dexamethasone concentration in lavage; (G) IGF concentration in cartilage lysate; (H) IGF concentration in meniscus lysate; (I) IGF concentration in ligament lysate; (J) IGF concentration in patella plus surrounding synovium lysate; (K) IGF concentration in serum; and (L) IGF concentration in lavage. Immediate time point samples are plotted at 0.1 hr. Data are mean±SEM. Missing points were below the limit of detection.

FIG. 16 is a graph of sulfated matrix biosynthesis measured by ³⁵S-sulfate incorporation for 4 and 12 day (4 D, white bars, and 12 D, black bars, respectively) treatments with GF-Fus1, GF-Fus3-His, or GF-Fus3 in the presence of cytokines (Disease). Cytokine treatment alone (Disease) included as a control. GF-Fus3 stimulated equivalent cartilage matrix synthesis to GF-Fus3-His. Data are mean±SEM.

FIG. 17 depicts two gel images showing fusion protein stability in synovial fluid from a 63 year old female with grade 1 cartilage (17A) and a 76 year old female with grade 3 cartilage (17B). The five lanes on the left were loaded with 45 ng of GF-Fus3 after incubation in synovial fluid at 37° C. for the indicated times. The five lanes on the right were loaded with stock GF-Fus3 standards (ng) which were not incubated in synovial fluid. A faint band at less than 7.5 kDa showed minimal protein degradation that was equal or less than 1 ng (i.e., less than 2% of loaded protein was degraded).

### DETAILED DESCRIPTION

The present disclosure provides fusion proteins comprising a first domain that specifically binds to the extracellular domain of a growth factor receptor (e.g., IGF-1 receptor), and a second domain that specifically binds to a cartilage matrix component (e.g., proteoglycan subunits such as a sulfated glycosaminoglycan (sGAG), a chondroitin sulfate and a collagen), and pharmaceutical compositions comprising these fusion proteins. Methods of treating musculoskeletal diseases, e.g., arthritis (e.g., osteoarthritis), traumatic joint injury, and related conditions using the fusion proteins and pharmaceutical composition disclosed herein are also provided.

### I. Definitions

As used herein the terms “Long [R³]-IGF-1,” “LR3” “IGF(LR3)” and “GF-Fus1” are used synonymously to refer to the IGF-1 variant polypeptide having the amino acid sequence: FPAMPLSSLFVNGPRTLCGAELVDALQFVCGDRGFYFNKPTGYGSSSRRA PQTGIVDECCFRSCDLRRLEMYCAPLKPAKSA (SEQ ID NO:1)

The terms “polypeptide” and “protein” are used interchangeably to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational and post-translational (C-terminal peptide cleavage) modifications of the polypeptide, such as, for example, disulfide-bond formation, glycosylation, acetylation, phosphorylation, proteolytic cleavage (e.g., cleavage by furins or metalloproteases), and the like. Furthermore, for purposes of the present disclosure, the terms “polypeptide” and “protein” include variants and derivatives with modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e g, similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

The term “substantial identity” as used herein denotes a characteristic of a polynucleotide or amino acid sequence, wherein the polynucleotide or amino acid comprises a sequence that has at least 85% sequence identity, preferably at least 90% to 95% sequence identity, more usually at least 99% sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan.

The term “substantially homologous” refers to sequences that are at least 90%, at least 95% identical, at least 96%, identical at least 97% identical, at least 98% identical or at least 99% identical. Homologous sequences can be the same functional gene in different species. Determination of homologs of the genes or peptides of the present invention can be easily ascertained by the skilled artisan.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), by the homology alignment algorithm of Needleman and Wunsch (J. Mol. Biol. 48:443-53 (1970)), by the search for similarity method of Pearson and Lipman (Proc. Natl. Acad. Sci. USA 85:2444-48 (1988)), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendrogram showing the clustering relationships used to create the alignment. PILEUP uses a simplification of the progressive alignment method of Feng and Doolittle (J. Mol. Evol. 25:351-60 (1987)). The method used is similar to the method described by Higgins and Sharp (Comput. Appl. Biosci. 5:151-53 (1989)). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990)). (See also Zhang et al., Nucleic Acid Res. 26:3986-90 (1998); Altschul et al., Nucleic Acid Res. 25:3389-402 (1997)). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992)) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

“Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to penetrate the BBB). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In certain embodiments, one can select the amino acid which will substitute an existing amino acid based on the location of the existing amino acid, i.e. its exposure to solvents (i.e. if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. Mol Biol, 1999, 217, 721-739 and Taylor et al, J. Theor. Biol. 119(1986); 205-218 and S. French and B. Robson, J. Mol. Evol. 19(1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

The term “derivative” as used herein refers to polypeptides which have been chemically modified, for example but not limited to by techniques such as ubiquitination, labeling, pegylation (derivatization with polyethylene glycol), lipidation, glycosylation, or addition of other molecules. A molecule is also a “derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990), incorporated herein, by reference, in its entirety.

The term “insertions” or “deletions” are typically in the range of about 1 to 5 amino acids. The variation allowed can be experimentally determined by producing the peptide synthetically while systematically making insertions, deletions, or substitutions of nucleotides in the sequence using recombinant DNA techniques.

The term “substitution” when referring to a peptide, refers to a change in an amino acid for a different entity, for example another amino acid or amino-acid moiety. Substitutions can be conservative or non-conservative substitutions.

By “covalently bonded” is meant joined either directly or indirectly (e.g., through a linker) by a covalent chemical bond.

The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. Fusion proteins can include an epitope tag or a half-life extender. Epitope tags include biotin, FLAG tag, c-myc, hemaglutinin, His₆, digoxigenin, FITC, Cy3, Cy5, green fluorescent protein, V5 epitope tags, GST, β-galactosidase, AU1, AUS, and avidin. Half-life extenders include Fc domain and serum albumin

The terms “subject” and “individual” and “patient” are used interchangeably herein, and refer to an animal, for example a human or non-human animal (e.g., a mammal), to whom treatment, including prophylactic treatment, with a pharmaceutical composition as disclosed herein, is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dogs, rodents (e.g. mouse or rat), guinea pigs, goats, pigs, cats, rabbits, cows, and non-mammals such as chickens, amphibians, reptiles etc. In one embodiment, the subject is human. In another embodiment, the subject is an experimental animal or animal substitute as a disease model.

“Treating” a disease or condition in a subject or “treating” a patient having a disease or condition refers to subjecting the individual to a pharmaceutical treatment, e.g., the administration of a drug, such that at least one symptom of the disease or condition is decreased, stabilized, or prevented.

By “specifically binds” or “specific binding” is meant a compound or antibody that recognizes and binds a desired polypeptide but that does not substantially recognize and bind other molecules in a sample, for example, a biological sample, which naturally includes a polypeptide of the invention. Specific binding can be characterized by a dissociation constant of at least about 1×10⁻⁶M or smaller. In other embodiments, the dissociation constant is at least about 1×10⁻⁷ M, 1×10⁻⁸ M, or 1×10⁻⁹ M. Methods for determining whether two molecules specifically bind are well known in the art and include, for example, equilibrium dialysis, surface plasmon resonance, and the like.

The term “readout” as used herein refers to any qualitative or quantitative measurement. In certain embodiments, the readout is a qualitative measurement. In certain embodiments, the readout is a quantitative measurement.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims Other features and advantages of the invention will be apparent from the following Detailed Description, the drawings, and the claims.

### II. Fusion Proteins

In one aspect, the present disclosure provides fusion proteins comprising a first domain that specifically binds to the extracellular domain of a growth factor receptor, and a second domain that specifically binds to a cartilage matrix component.

The first domain can target any desired receptor (e.g., a growth factor receptor). In certain embodiments, the first domain targets a growth factor receptor implicated in musculoskeletal disease (e.g., the IGF-1 receptor). The first domain can comprise a natural or artificial ligand for the growth factor receptor. The first domain can be an agonist or antagonist of the targeted growth factor receptor, as desired. In certain embodiments, the first domain comprises an IGF-1 receptor ligand (e.g., a human IGF-1 receptor ligand). In one particular embodiment, the first domain comprises the human IGF-1 sequence. In one particular embodiment, the first domain comprises a Long [R³]-IGF-1 sequence (e.g., the human Long [R³]-IGF-1 sequence set forth in SEQ ID NO:1). In one particular embodiment, the first domain comprises a polypeptide having at least 80% amino acid identity (e.g., at least 81, 82, 83, 84, 85, 86, 87, 88, 89, 90, 91, 92, 93, 94, 95, 96, 97, 98, or 99% identity), with the human Long [R³]-IGF-1 sequence set forth in SEQ ID NO:1.

The second type of domain can target any cartilage matrix component, including without limitation, sGAG (e.g., heparan sulfate, chondroitin, dermatan sulfate, and keratan sulfate) and/or collagen or hyaluronic acid. Suitable sGAG binding domains that can be used in the second domain include without limitation the sGAG binding domain of: epidermal growth factor (EGF), proline-arginine-rich end leucine-rich repeat protein (PRELP), chondroadherin, oncostatin M, collagen IX, BMP-4, fibronectin, RAND1, RAND2, RAND3, RAND4, RAND5, RAND6, AKK15, RLR22, R1Q17, SEK20, ARK24, AKK24, AL1, AL2, AL3, LGT25, Pep184, Pep186, Pep185, Pep239, Pep246, ATIII, or FibBeta. Suitable collagen binding domains that can be used in the second domain include without limitation the collagen binding domain of: thrombospondin, matrilin, cartilage oligomeric matrix protein, PRELP, chondroadherin, fibromodulin, decorin, or asporin. Exemplary sGAG and collagen binding domains are set forth in Tables 1 and 2 herein.

In certain embodiments, the second domain is fused to the N-terminus of the first domain. In other embodiments, the second domain is fused to the C-terminus of the first domain. The fusion proteins may further comprise a linker between the domains. In certain embodiments, the fusion proteins comprise more than one domain that specifically binds to a cartilage matrix component. The more than one cartilage matrix binding domains may comprise the same binding domains or alternatively may each comprise a different type of cartilage matrix binding (i.e., second) domain.

In certain embodiments, the second domain comprises a sGAG binding domain having an amino acid sequence selected from the group consisting of SEQ ID NOs: 2-13, and 54-70 (see Table 1). In certain embodiments, the second domain comprises a sGAG binding domain having at least 80% amino acid identity (e.g., at least 81, 82, 83, 84, 85, 86, 87, 88, 89, 90, 91, 92, 93, 94, 95, 96, 97, 98, or 99% identity) to an amino acid sequence selected from the group consisting of SEQ ID NO: 2-13, and 54-70 (see Table 1).

In certain embodiments, the second domain comprises a collagen binding domain having an amino acid sequence selected from the group consisting of SEQ ID NO: 14-16, and 21-27 (see Table 2). In certain embodiments, the second domain comprises a collagen binding domain having at least 80% amino acid identity (e.g., at least 81, 82, 83, 84, 85, 86, 87, 88, 89, 90, 91, 92, 93, 94, 95, 96, 97, 98, or 99% identity) to an amino acid sequence selected from the group consisting of SEQ ID NO: 14-16, and 21-27 (see Table 2).

In certain embodiments, the first binding domain binds to a receptor (e.g., a growth factor receptor) with a Kd of less than 1000 nM (e.g., less than 100, 10, 1, 0.1, 0.01, 0.001, or 0.0001 nM). Note that a lower Kd corresponds to a higher binding affinity. In certain embodiments, the second binding domain binds to a cartilage matrix component (e.g., sGAG or collagen) with a Kd of less than 1000 nM (e.g., less than 100, 10, 1, 0.1, 0.01, 0.001, or 0.0001 nM).

In certain embodiments, the fusion protein comprises an amino acid sequence selected from the group consisting of SEQ ID NO: 17-20, 28-53, 71-87 (see Table 3). In one particular embodiment, the fusion protein comprises the amino acid sequence set forth in SEQ ID NO: 18. In one particular embodiment, the fusion protein consists of the amino acid sequence set forth in SEQ ID NO: 18. In certain embodiments, the fusion protein comprises an amino acid sequence having at least 80% amino acid identity (e.g., at least 81, 82, 83, 84, 85, 86, 87, 88, 89, 90, 91, 92, 93, 94, 95, 96, 97, 98, or 99% identity) to an amino acid sequence selected from the group consisting of SEQ ID NO: 17-20, 28-53, 71-87 (see Table 3). In certain embodiments, the fusion protein comprises a Histidine tag. As used herein, a histidine tag, or 6× histidine tag, comprises a peptide with the sequence GGSGGHHHHHH (SEQ ID NO:89) fused to the c-terminus of the fusion protein.

In certain embodiments, the fusion proteins disclosed herein are retained within cartilage tissue of a joint for a time period of at least 8 days (e.g., 8 days, 9 days, 10 days, 11 days, or 12 days) after injection into an intra-articular space (e.g. synovial fluid) of a joint of a mammal so that a detectable level of the fusion protein can be found in a biopsy of cartilage tissue taken at said time period. In certain embodiments, the detectable level (amount) of fusion protein retained in the cartilage tissue can be at least about 5 (e.g., about 10, 20, 30, 40, or 50 pmol/g) of tissue.

In certain embodiments, when administered to a joint, the fusion proteins disclosed herein result in a reduction in loss of sGAG from the joint cartilage tissue, when compared to loss of sGAG in cartilage tissue of a matched control joint that has been injected with an innocuous control protein (such as serum albumin) In certain embodiments, when administered to an injured joint, the fusion proteins disclosed herein result in an increase in production of sGAG in the joint cartilage tissue, when compared to production of sGAG in cartilage tissue of an injured joint that has been injected with the innocuous control protein. In certain embodiments, when administered to a joint, the fusion proteins disclosed herein result an increase in the content of sGAG in the cartilage tissue, when compared to the content of sGAG in cartilage tissue of an injured joint that has been injected with the innocuous control protein.

TABLE 1
Exemplary Glycosaminoglycan (GAG) Binding Domain Sequences

| Name | Heparin Binding Domain Sequence | SEQ ID NO: |
|---|---|---|
| PRELP | QPTRRPRPGTGPGRRPRPRPRP | 2 |
| BMP-4 | RKKNPNCRRH | 3 |
| Fibronectin | WQPPRARI | 4 |
| Oncostatin M | LRKGVRRTRPSRKGKRLMTRG | 5 |
| RAND1 | AVKRRPRFPAVKRRPRFP | 6 |
| RAND2 | AKRRAARAAKRRAARAAKRRAARA | 7 |
| Chondroadherin | KFPTKRSKKAGRH | 8 |
| RAND3 | SKKARAGTGAKKARA | 9 |
| RAND4 | ARKKAAKAGTGARKKAAKA | 10 |
| Collagen IX | AVKRRPRFPVNSNSNGGNE | 11 |
| RAND5 | AKKARAAKKARAAKKARA | 12 |
| RAND6 | ARKKAAKAARKKAAKASRKKAAKA | 13 |
| AKK15 | AKKQRFRHRNRKGYR | 54 |
| RLR22 | RLRAQSRQRSRPGRWHKVSVRW | 55 |
| R1Q17 | RIQNLLKITNLRIKFVKL | 56 |
| SEK20 | SEKTLRKWLKMFKKRQLELY | 57 |
| ARK24 | ARKKAAKAARKKAAKAARKKAAKA | 58 |
| AKK24 | AKKARAAKKARAAKKARAAKKARA | 59 |
| AL1 | RPLREKMKPERRRPKGRGKRRREKQRPT | 60 |
| AL2 | RRPKGRGKRRREKQRPTDAHL | 61 |
| AL3 | QPTRRPRPGTGPGRRPRPRPRPTPSAPQPTRRPRPGTGP | 62 |
| | GRRPRPRPRP | |
| LGT25 | LGTRLRAQSRQRSRPGRWHKVSVRW | 63 |
| Pep184 | SPWSEWTSSSTS | 64 |
| Pep186 | GPWSPWDISSVT | 65 |
| Pep185 | SHWSPWSSSSVT | 66 |
| Pep239 | SHWSPWSS | 67 |
| Pep246 | WSPWSSSSVT | 68 |
| ATIII | AKLNSRLYRKANKSSKLVSANRLFGDK | 69 |
| FibBeta | QGVNDNEEGFFSARGHRPLDKKREEAPSLRPAPPP | 70 |

“RAND” as used herein describes random generation of sequences with specific patterns of positive charges. The above proteins are described at least in, e.g., Martino et al., Science v343, 885 (2014); Tillgren et al., J. Biol Chem. v284 No. 42 (2009); Andersson et al., Eur. J. Biochem. 271, 1219-1226 (2004); Hileman et al., BioEssays 20:156-167, (1998); and Guo et al., PNAS v89, 3040-3044 (1992).

TABLE 2
Exemplary Collagen Binding Domain Sequences

| Name | Collagen Binding Domain | SEQ ID NO: |
|---|---|---|
| CNA-35 | ITSGNKSTNVTVHKSEAGTSSVFYYKTGDMLPEDT | 14 |
| | THVRWFLNINNEKRYVSKDITIKDQIQGGQQLDLST | |
| | LNINVTGTHSNYYSGPNAITDFEKAFPGSKITVDNT | |
| | KNTIDVTIPQGYGSLNSFSINYKTKITNEQQKEFVN | |
| | NSQAWYQEHGKEEVNGKAFNHTVHN | |
| CNA-344 | RDISSTNVTDLTVSPSKIEDGGKTTVKMTFDDKNG | 15 |
| | KIQNGDTIKVAWPTSGTVKIEGYSKTVSLTVKGEQ | |
| | VGQAVITPDGATITFNDKVEKLSDVSGFAEFEVQG | |
| | RNLTQTNTSDDKVATITSGNKSTNVTVHKSEAGTS | |
| | SVFYYKTGDMLPEDTTHVRWFLNINNEKRYVSKDI | |
| | TIKDQIQGGQQLDLSTLNINVTGTHSNYYSGPNAIT | |
| | DFEKAFPGSKITVDNTKNTIDVTIPQGYGSLNSFSIN | |
| | YKTKITNEQQKEFVNNSQAWYQEHGKEEVNGKAF | |
| | NHTVHNINANAGIEGTVKGELKVLKQDKDTKA | |
| Thrombospondin | KVSCPIMPCSNATVPDGECCPRCWPSDSADDGWSP | 16 |
| | WSEWTSCSTSCGNGIQQRGRSCDSLNNRCEGSSVQ | |
| | TRTCHIQECDK | |
| Decorin | CPFRCQCHLRVVQCSDLGLDKVPKDLPPDTTLLDL | 21 |
| | QNNKITEIKDGDFKNLKNLHALILVNNKISKVSPGA | |
| | FTPLVKLERLYLSKNQLKELPEKMPKTLQELRAHE | |
| | NEITKVRKVTFNGLNQMIVIELGTNPLKSSGIENGA | |
| | FQGMKKLSYIRIADTNITSIPQGLPPSLTELHLDGNK | |
| | ISRVDAASLKGLNNLAKLGLSFNSISAVDNGSLANT | |
| | PHLRELHLDNNKL | |
| Asporin | LFPMCPFGCQCYSRVVHCSDLGLTSVPTNIPFDTRM | 22 |
| | LDLQNNKIKEIKENDFKGLTSLYGLILNNNKLTKIH | |
| | PKAFLTTKKLRRLYLSHNQLSEIPLNLPKSLAELRIH | |
| | ENKVKKIQKDTFKGMNALHVLEMSANPLDNNGIE | |
| | PGAFEGVTVFHIRIAEAKLTSVPKGLPPTLLELHLD | |
| | YNKISTVELEDFKRYKELQRLGLGNNKITDIENGSL | |
| | ANIPRVREIHLENNKLKK | |
| Chondroadherin | KLLNLQRNNFPVLAANSFRAMPNLVSLHLQHCQIR | 23 |
| | EVAAGAFRGLKQLIYLYLSHNDIRVLRAGAFDDLT | |
| | ELTYLYLDHNKVTELPRGLLSPLVNLFILQLNNNKI | |
| | RELRAGAFQGAKDLRWLYLSENALSSLQPGALDD | |
| | VENLAKFHVDRNQLSSYPSAALSKLRVVEELKLSH | |
| | NPLKSIPDNAFQSFGRYLETLWLDNTNLEKFSDGAF | |
| | LGVTTLKHVHLENNRLNQLPSNFPFDSLETLALTN | |
| | NPWKCTCQLRGLRRWLEAKASRPDATCASPAKFK | |
| | GQHIRDTDAFRSCK | |
| Matrilin | RPLDLVFIIDSSRSVRPLEFTKVKTFVSRIIDTLDIGP | 24 |
| | ADTRVAVVNYASTVKIEFQLQAYTDKQSLKQAVG | |
| | RITPLSTGTMSGLAIQTAMDEAFTVEAGAREPSSNIP | |
| | KVAIIVTDGRPQDQVNEVAARAQASGIELYAVGVD | |
| | RADMASLKMMASEPLEEHVFYVETYGVIEKLSSRF | |
| | QETFCALDPCVLGTHQCQHVCISDGEGKHHCECSQ | |
| | GYTLNADKKTCSALDRCALNTHGCEHICVNDRSGS | |
| | YHCECYEGYTLNEDRKTCSAQDKCALGTHGCQHI | |
| | CVNDRTGSHHCECYEGYTLNADKKTCSVRDKCAL | |
| | GSHGCQHICVSDGAASYHCDCYPGYTLNEDKKT | |
| Fibromodulin | DCPQECDCPPNFLTAMYCDNRNLKYLPFVPSRMK | 25 |
| | YVYFQNNQITSIQEGVFDNATGLLWIALHGNQITSD | |
| | KVGRKVFSKLRHLERLYLDHNNLTRMPGPLPRSLR | |
| | ELHLDHNQISRVPNNALEGLENLTALYLQHDEIQEV | |
| | GSSMRGLRSLILLDLSYNHLRKVPDGLPSALEQLY | |
| | MEHNNVYTVPDSYFRGAPKLLYVRLSHNSLTNNG | |
| | LASNTFNSSSLLELDLSYNQLQKIPPVNTNLENLYL | |
| | QGNRINEFSISSFCTVVDVVNFSKLQVVRLDGNEI | |
| PRELP | DCPRECYCPPDFPSALYCDSRNLRKVPVIPPRIHYL | 26 |
| | YLQSNFITELPVESFQNATGLRWINLDNNRIRKIDQ | |
| | RVLEKLPGLVFLYMEKNQLEEVPSALPRNLEQLRL | |
| | SQNHISRIPPGVFSKLENLLLLDLQHNRLSDGVFKP | |
| | DTFHGLKNLMQLNLAHNILRKMPPRVPTAIHQLYL | |
| | DSNKIETIPNGYFKSFPNLAFIRLNYNKLTDRGLPKN | |
| | SFNISNLLVLHLSHNRISSVPAINNRLEHLYLNNNSI | |
| | EKINGTQICPNDLVAFHDFSSDLENVPHLRYLRLDG | |
| | NYL | |
| COMP | DLGPQMLRELQETNAALQDVRELLRQQVREITFLK | 27 |
| (cartilage | NTVMECDACGMQQSVRTGLPSVRPLLHCAPGFCFP | |
| oligomeric | GVACIQTESGARCGPCPAGFTGNGSHCTDVNECNA | |
| protein) | HPCFPRVRCINTSPGFRCEACPPGYSGPTHQGVGLA | |
| | FAKANKQVCTDINECETGQHNCVPNSVCINTRGSF | |
| | QCGPCQPGFVGDQASGCQRRAQRFCPDGSPSECHE | |
| | HADCVLERDGSRSCVCAVGWAGNGILCGRDTDLD | |
| | GFPDEKLRCPERQCRKDNCVTVPNSGQEDVDRDGI | |
| | GDACDPDADGDGVPNEKDNCPLVRNPDQRNTDED | |
| | KWGDACDNCRSQKNDDQKDTDQDGRGDACDDDI | |
| | DGDRIRNQADNCPRVPNSDQKDSDGDGIGDACDN | |
| | CPQKSNPDQADVDHDFVGDACDSDQDQDGDGHQ | |
| | DSRDNCPTVPNSAQEDSDHIDGQGDACDDDDDNDG | |
| | VPDSRDNCRLVPNPGQEDADRDGVGDVCQDDFDA | |
| | DKVVDKIDVCPENAEVTLTDFRAFQTVVLDPEGDA | |
| | QIDPNWVVLNQGREIVQTMNSDPGLAVGYTAFNG | |
| | VDFEGTFHVNTVTDDDYAGFIFGYQDSSSFYVVM | |
| | WKQMEQTYWQANPFRAVAEPGIQLKAVKSSTGPG | |
| | EQLRNALWHTGDTESQVRLLWKDPRNVGWKDKK | |
| | SYRWFLQHRPQVGYIRVRFYEGPELVADSNVVLDT | |
| | TMRGGRLGVFCFSQENIIWANLRYRCNGE | |

TABLE 3
Exemplary Fusion Protein Sequences

| Name | Collagen Binding Domain | SEQ ID NO: |
|---|---|---|
| C-terminal | MAWRLWWLLLLLLLLWPMVWAFPAMPLSSLFVN | 17 |
| fusion IGF- | GPRTLCGAELVDALQFVCGDRGFYFNKPTGYGSSS | |
| 1(LR3)-PRELP | RRAPQTGIVDECCFRSCDLRRLEMYCAPLKPAKSA | |
| with signal | GGGGSGGGGSGGGGSQPTRRPRPGTGPGRRPRPRP | |
| sequence | RP | |
| GF-Fus3 (C- | FPAMPLSSLFVNGPRTLCGAELVDALQFVCGDRGF | 18 |
| terminal fusion | YFNKPTGYGSSSRRAPQTGIVDECCFRSCDLRRLEM | |
| IGF-1(LR3)- | YCAPLKPAKSAGGGGSGGGGSGGGGSQPTRRPRPG | |
| PRELP) | TGPGRRPRPRPRP | |
| N-terminal | MAWRLWWLLLLLLLLWPMVWAQPTRRPRPGTGP | 19 |
| fusion IGF- | GRRPRPRPRPGGGGSGGGGSGGGGSFPAMPLSSLF | |
| 1(LR3)-PRELP | VNGPRTLCGAELVDALQFVCGDRGFYFNKPTGYG | |
| with signal | SSSRRAPQTGIVDECCFRSCDLRRLEMYCAPLKPAK | |
| sequence | SA | |
| N-terminal | QPTRRPRPGTGPGRRPRPRPRPGGGGSGGGGSGGG | 20 |
| fusion IGF- | GSFPAMPLSSLFVNGPRTLCGAELVDALQFVCGDR | |
| 1(LR3)-PRELP | GFYFNKPTGYGSSSRRAPQTGIVDECCFRSCDLRRL | |
| | EMYCAPLKPAKSA | |
| C-terminal | MAWRLWWLLLLLLLLWPMVWAFPAMPLSSLFVN | 28 |
| direct fusion | GPRTLCGAELVDALQFVCGDRGFYFNKPTGYGSSS | |
| IGF-1(LR3)- | RRAPQTGIVDECCFRSCDLRRLEMYCAPLKPAKSA | |
| CHAD with | KFPTKRSKKAGRH | |
| signal sequence | | |
| C-terminal | FPAMPLSSLFVNGPRTLCGAELVDALQFVCGDRGF | 29 |
| direct fusion | YFNKPTGYGSSSRRAPQTGIVDECCFRSCDLRRLEM | |
| IGF-1(LR3)- | YCAPLKPAKSAKFPTKRSKKAGRH | |
| CHAD | | |
| N-terminal | MAWRLWVVLLLLLLLLWPMVVVAKFPTKRSKKAGR | 30 |
| direct fusion | HFPAMPLSSLFVNGPRTLCGAELVDALQFVCGDRG | |
| CHAD-IGF- | FYFNKPTGYGSSSRRAPQTGIVDECCFRSCDLRRLE | |
| 1(LR3) with | MYCAPLKPAKSA | |
| signal sequence | | |
| N-terminal | KFPTKRSKKAGRHFPAMPLSSLFVNGPRTLCGAEL | 31 |
| direct fusion | VDALQFVCGDRGFYFNKPTGYGSSSRRAPQTGIVD | |
| CHAD-IGF- | ECCFRSCDLRRLEMYCAPLKPAKSA | |
| 1(LR3) | | |
| GF-Fus2 (N- | QPTRRPRPGTGPGRRPRPRPRPGPETLCGAXLVDAL | 32 |
| terminal Prelp | QFVCGDRGFYFNKPTGYGSSSRRAPQTGIVDXCCF | |
| HB domain | RSCDLRRLEMYCAPLKPAKSA | |
| fused to wild- | | |
| type IGF) | | |
| GF-Fus4 (IGF- | FPAMPLSSLFVNGPRTLCGAELVDALQFVCGDRGF | 33 |
| 1(LR3) fused to | YFNKPTGYGSSSRRAPQTGIVDECCFRSCDLRRLEM | |
| Collagen IX HB | YCAPLKPAKSAGGGGSGGGGSGGGGSASAVKRRP | |
| domain) | RFPVNSNSNGGNE | |
| GF-Fus5 (IGF- | FPAMPLSSLFVNGPRTLCGAELVDALQFVCGDRGF | 34 |
| 1(LR3) fused to | YFNKPTGYGSSSRRAPQTGIVDECCFRSCDLRRLEM | |
| CNA35 CB | YCAPLKPAKSAGGGGSGGGGSGGGGSASITSGNKS | |
| domain) | TNVTVHKSEAGTSSVFYYKTGDMLPEDTTHVRWF | |
| | LNINNEKRYVSKDITIKDQIQGGQQLDLSTLNINVT | |
| | GTHSNYYSGPNAITDFEKAFPGSKITVDNTKNTIDV | |
| | TIPQGYGSLNSFSINYKTKITNEQQKEFVNNSQAWY | |
| | QEHGKEEVNGKAFNHTVHN | |
| GF-Fus6 (IGF- | FPAMPLSSLFVNGPRTLCGAELVDALQFVCGDRGF | 35 |
| 1(LR3) fused to | YFNKPTGYGSSSRRAPQTGIVDECCFRSCDLRRLEM | |
| CNA344 CB | YCAPLKPAKSAGGGGSGGGGSGGGGSASRDISSTN | |
| domain) | VTDLTVSPSKIEDGGKTTVKMTFDDKNGKIQNGDT | |
| | IKVAWPTSGTVKIEGYSKTVSLTVKGEQVGQAVITP | |
| | DGATITFNDKVEKLSDVSGFAEFEVQGRNLTQTNT | |
| | SDDKVATITSGNKSTNVTVHKSEAGTSSVFYYKTG | |
| | DMLPEDTTHVRWFLNINNEKRYVSKDITIKDQIQG | |
| | GQQLDLSTLNINVTGTHSNYYSGPNAITDFEKAFPG | |
| | SKITVDNTKNTIDVTIPQGYGSLNSFSINYKTKITNE | |
| | QQKEFVNNSQAWYQEHGKEEVNGKAFNHTVHNIN | |
| | ANAGIEGTVKGELKVLKQDKDTKA | |
| IGF-1(LR3) | FPAMPLSSLFVNGPRTLCGAELVDALQFVCGDRGF | 36 |
| fused to BMP-4 | YFNKPTGYGSSSRRAPQTGIVDECCFRSCDLRRLEM | |
| HB domain | YCAPLKPAKSAGGGGSGGGGSGGGGSASRKKNPN | |
| | CRRH | |
| IGF-1(LR3) | FPAMPLSSLFVNGPRTLCGAELVDALQFVCGDRGF | 37 |
| fused to | YFNKPTGYGSSSRRAPQTGIVDECCFRSCDLRRLEM | |
| Fibronectin HB | YCAPLKPAKSAGGGGSGGGGSGGGGSASWQPPRA | |
| domain | RI | |
| IGF-1(LR3) | FPAMPLSSLFVNGPRTLCGAELVDALQFVCGDRGF | 38 |
| fused to | YFNKPTGYGSSSRRAPQTGIVDECCFRSCDLRRLEM | |
| Oncostatin M | YCAPLKPAKSAGGGGSGGGGSGGGGSASLRKGVR | |
| HB domain | RTRPSRKGKRLMTRG | |
| IGF-1(LR3) | FPAMPLSSLFVNGPRTLCGAELVDALQFVCGDRGF | 39 |
| fused to | YFNKPTGYGSSSRRAPQTGIVDECCFRSCDLRRLEM | |
| RAND1 HB | YCAPLKPAKSAGGGGSGGGGSGGGGSAVKRRPRF | |
| domain | PAVKRRPRFP | |
| IGF-1(LR3) | FPAMPLSSLFVNGPRTLCGAELVDALQFVCGDRGF | 40 |
| fused to | YFNKPTGYGSSSRRAPQTGIVDECCFRSCDLRRLEM | |
| RAND2 HB | YCAPLKPAKSAGGGGSGGGGSGGGGSAKRRAARA | |
| domain | AKRRAARAAKRRAARA | |
| IGF-1(LR3) | FPAMPLSSLFVNGPRTLCGAELVDALQFVCGDRGF | 41 |
| fused to | YFNKPTGYGSSSRRAPQTGIVDECCFRSCDLRRLEM | |
| Chondroadherin | YCAPLKPAKSAGGGGSGGGGSGGGGSASKFPTKRS | |
| HB domain | KKAGRH | |
| IGF-1(LR3) | FPAMPLSSLFVNGPRTLCGAELVDALQFVCGDRGF | 42 |
| fused to | YFNKPTGYGSSSRRAPQTGIVDECCFRSCDLRRLEM | |
| RAND3 HB | YCAPLKPAKSAGGGGSGGGGSGGGGSSKKARAGT | |
| domain | GAKKARA | |
| IGF-1(LR3) | FPAMPLSSLFVNGPRTLCGAELVDALQFVCGDRGF | 43 |
| fused to | YFNKPTGYGSSSRRAPQTGIVDECCFRSCDLRRLEM | |
| RAND4 HB | YCAPLKPAKSAGGGGSGGGGSGGGGSARKKAAKA | |
| domain | GTGARKKAAKA | |
| IGF-1(LR3) | FPAMPLSSLFVNGPRTLCGAELVDALQFVCGDRGF | 44 |
| fused to | YFNKPTGYGSSSRRAPQTGIVDECCFRSCDLRRLEM | |
| RANDS HB | YCAPLKPAKSAGGGGSGGGGSGGGGSAKKARAAK | |
| domain | KARAAKKARA | |
| IGF-1(LR3) | FPAMPLSSLFVNGPRTLCGAELVDALQFVCGDRGF | 45 |
| fused to | YFNKPTGYGSSSRRAPQTGIVDECCFRSCDLRRLEM | |
| RAND6 HB | YCAPLKPAKSAGGGGSGGGGSGGGGSARKKAAKA | |
| domain | ARKKAAKASRKKAAKA | |
| IGF-1(LR3) | FPAMPLSSLFVNGPRTLCGAELVDALQFVCGDRGF | 46 |
| fused to | YFNKPTGYGSSSRRAPQTGIVDECCFRSCDLRRLEM | |
| thrombospondin | YCAPLKPAKSAGGGGSGGGGSGGGGSASKVSCPIM | |
| CB domain | PCSNATVPDGECCPRCWPSDSADDGWSPWSEWTS | |
| | CSTSCGNGIQQRGRSCDSLNNRCEGSSVQTRTCHIQ | |
| | ECDK | |
| IGF-1(LR3) | FPAMPLSSLFVNGPRTLCGAELVDALQFVCGDRGF | 47 |
| fused to Decorin | YFNKPTGYGSSSRRAPQTGIVDECCFRSCDLRRLEM | |
| CB domain | YCAPLKPAKSAGGGGSGGGGSGGGGSASCPFRCQ | |
| | CHLRVVQCSDLGLDKVPKDLPPDTTLLDLQNNKIT | |
| | EIKDGDFKNLKNLHALILVNNKISKVSPGAFTPLVK | |
| | LERLYLSKNQLKELPEKMPKTLQELRAHENEITKV | |
| | RKVTFNGLNQMIVIELGTNPLKSSGIENGAFQGMK | |
| | KLSYIRIADTNITSIPQGLPPSLTELHLDGNKISRVDA | |
| | ASLKGLNNLAKLGLSFNSISAVDNGSLANTPHLREL | |
| | HLDNNKL | |
| IGF-1(LR3) | FPAMPLSSLFVNGPRTLCGAELVDALQFVCGDRGF | 48 |
| fused to Asporin | YFNKPTGYGSSSRRAPQTGIVDECCFRSCDLRRLEM | |
| CB domain | YCAPLKPAKSAGGGGSGGGGSGGGGSASLFPMCPF | |
| | GCQCYSRVVHCSDLGLTSVPTNIPFDTRMLDLQNN | |
| | KIKEIKENDFKGLTSLYGLILNNNKLTKIHPKAFLTT | |
| | KKLRRLYLSHNQLSEIPLNLPKSLAELRIHENKVKKI | |
| | QKDTFKGMNALHVLEMSANPLDNNGIEPGAFEGV | |
| | TVFHIRIAEAKLTSVPKGLPPTLLELHLDYNKISTVE | |
| | LEDFKRYKELQRLGLGNNKITDIENGSLANIPRVREI | |
| | HLENNKLKK | |
| IGF-1(LR3) | FPAMPLSSLFVNGPRTLCGAELVDALQFVCGDRGF | 49 |
| fused to | YFNKPTGYGSSSRRAPQTGIVDECCFRSCDLRRLEM | |
| Chondroadherin | YCAPLKPAKSAGGGGSGGGGSGGGGSASKLLNLQ | |
| CB domain | RNNFPVLAANSFRAMPNLVSLHLQHCQIREVAAGA | |
| | FRGLKQLIYLYLSHNDIRVLRAGAFDDLTELTYLYL | |
| | DHNKVTELPRGLLSPLVNLFILQLNNNKIRELRAGA | |
| | FQGAKDLRWLYLSENALSSLQPGALDDVENLAKF | |
| | HVDRNQLSSYPSAALSKLRVVEELKLSHNPLKSIPD | |
| | NAFQSFGRYLETLWLDNTNLEKFSDGAFLGVTTLK | |
| | HVHLENNRLNQLPSNFPFDSLETLALTNNPWKCTC | |
| | QLRGLRRWLEAKASRPDATCASPAKFKGQHIRDTD | |
| | AFRSCK | |
| IGF-1(LR3) | FPAMPLSSLFVNGPRTLCGAELVDALQFVCGDRGF | 50 |
| fused to Matrilin | YFNKPTGYGSSSRRAPQTGIVDECCFRSCDLRRLEM | |
| CB domain | YCAPLKPAKSAGGGGSGGGGSGGGGSASRPLDLVF | |
| | IIDSSRSVRPLEFTKVKTFVSRIIDTLDIGPADTRVAV | |
| | VNYASTVKIEFQLQAYTDKQSLKQAVGRITPLSTGT | |
| | MSGLAIQTAMDEAFTVEAGAREPSSNIPKVAIIVTD | |
| | GRPQDQVNEVAARAQASGIELYAVGVDRADMASL | |
| | KMMASEPLEEHVFYVETYGVIEKLSSRFQETFCAL | |
| | DPCVLGTHQCQHVCISDGEGKHHCECSQGYTLNA | |
| | DKKTCSALDRCALNTHGCEHICVNDRSGSYHCECY | |
| | EGYTLNEDRKTCSAQDKCALGTHGCQHICVNDRT | |
| | GSHHCECYEGYTLNADKKTCSVRDKCALGSHGCQ | |
| | HICVSDGAASYHCDCYPGYTLNEDKKT | |
| IGF-1(LR3) | FPAMPLSSLFVNGPRTLCGAELVDALQFVCGDRGF | 51 |
| fused to | YFNKPTGYGSSSRRAPQTGIVDECCFRSCDLRRLEM | |
| Fibromodulin | YCAPLKPAKSAGGGGSGGGGSGGGGSASDCPQEC | |
| CB domain | DCPPNFLTAMYCDNRNLKYLPFVPSRMKYVYFQN | |
| | NQITSIQEGVFDNATGLLWIALHGNQITSDKVGRKV | |
| | FSKLRHLERLYLDHNNLTRMPGPLPRSLRELHLDH | |
| | NQISRVPNNALEGLENLTALYLQHDEIQEVGSSMR | |
| | GLRSLILLDLSYNHLRKVPDGLPSALEQLYMEHNN | |
| | VYTVPDSYFRGAPKLLYVRLSHNSLTNNGLASNTF | |
| | NSSSLLELDLSYNQLQKIPPVNTNLENLYLQGNRIN | |
| | EFSISSFCTVVDVVNFSKLQVVRLDGNEI | |
| IGF-1(LR3) | FPAMPLSSLFVNGPRTLCGAELVDALQFVCGDRGF | 52 |
| fused to PRELP | YFNKPTGYGSSSRRAPQTGIVDECCFRSCDLRRLEM | |
| CB domain | YCAPLKPAKSAGGGGSGGGGSGGGGSASDCPREC | |
| | YCPPDFPSALYCDSRNLRKVPVIPPRIHYLYLDCPRE | |
| | CYCPPDFPSALYCDSRNLRKVPVIPPRIHYLYLQSNF | |
| | ITELPVESFQNATGLRWINLDNNRIRKIDQRVLEKLP | |
| | GLVFLYMEKNQLEEVPSALPRNLEQLRLSQNHISRI | |
| | PPGVFSKLENLLLLDLQHNRLSDGVFKPDTFHGLK | |
| | NLMQLNLAHNILRKMPPRVPTAIHQLYLDSNKIETI | |
| | PNG | |
| | YFKSFPNLAFIRLNYNKLTDRGLPKNSFNISNLLVL | |
| | HLSHNRISSVPAINNRLEHLYLNNNSIEKINGTQICP | |
| | NDLVAFHDFSSDLENVPHLRYLRLDGNYL | |
| IGF-1(LR3) | FPAMPLSSLFVNGPRTLCGAELVDALQFVCGDRGF | 53 |
| fused to | YFNKPTGYGSSSRRAPQTGIVDECCFRSCDLRRLEM | |
| Cartilage | YCAPLKPAKSAGGGGSGGGGSGGGGSASDLGPQM | |
| Oligomeric | LRELQETNAALQDVRELLRQQVREITFLKNTVMEC | |
| Protein CB | DACGMQQSVRTGLPSVRPLLHCAPGFCFPGVACIQ | |
| domain | TESGARCGPCPAGFTGNGSHCTDVNECNAHPCFPR | |
| | VRCINTSPGFRCEACPPGYSGPTHQGVGLAFAKAN | |
| | KQVCTD | |
| | INECETGQHNCVPNSVCINTRGSFQCGPCQPGFVGD | |
| | QASGCQRRAQRFCPDGSPSECHEHADCVLERDGSR | |
| | SCVCAVGWAGNGILCGRDTDLDGFPDEKLRCPER | |
| | QCRKDNCVTVPNSGQEDVDRDGIGDACDPDADGD | |
| | GVPNEKDNCPLVRNPDQRNTDEDKWGDACDNCRS | |
| | QKNDDQKDTDQDGRGDA | |
| | CDDDIDGDRIRNQADNCPRVPNSDQKDSDGDGIGD | |
| | ACDNCPQKSNPDQADVDHDFVGDACDSDQDQDG | |
| | DGHQDSRDNCPTVPNSAQEDSDHDGQGDACDDDD | |
| | DNDGVPDSRDNCRLVPNPGQEDADRDGVGDVCQ | |
| | DDFDADKVVDKIDVCPENAEVTLTDFRAFQTVVLD | |
| | PEGDAQIDPNWVVLNQGREIVQTMNSDPGLAVGY | |
| | TAFNGVDFEGTFHVNTVTDDDYAGFIFGYQDSSSF | |
| | YVVMWKQMEQTYWQANPFRAVAEPGIQLKAVKS | |
| | STGPGEQLRNALWHTGDTESQVRLLWKDPRNVGW | |
| | KDKKSYRWFLQHRPQVGYIRVRFYEGPELVADSN | |
| | VVLDTTMRGGRLGVFCFSQENIIWANLRYRCNGE | |
| LR3_IGF_G4S3_AKK15 | FPAMPLSSLFVNGPRTLCGAELVDALQFVCGDRGF | 71 |
| | YFNKPTGYGSSSRRAPQTGIVDECCFRSCDLRRLEM | |
| | YCAPLKPAKSAGGGGSGGGGSGGGGSAKKQRFRH | |
| | RNRKGYR | |
| LR3_IGF_G4S3_RLR22 | FPAMPLSSLFVNGPRTLCGAELVDALQFVCGDRGF | 72 |
| | YFNKPTGYGSSSRRAPQTGIVDECCFRSCDLRRLEM | |
| | YCAPLKPAKSAGGGGSGGGGSGGGGSRLRAQSRQ | |
| | RSRPGRWHKVSVRW | |
| LR3_IGF_G4S3_R1Q17 | FPAMPLSSLFVNGPRTLCGAELVDALQFVCGDRGF | 73 |
| | YFNKPTGYGSSSRRAPQTGIVDECCFRSCDLRRLEM | |
| | YCAPLKPAKSAGGGGSGGGGSGGGGSRIQNLLKIT | |
| | NLRIKFVKL | |
| LR3_IGF_G4S3_SEK20 | FPAMPLSSLFVNGPRTLCGAELVDALQFVCGDRGF | 74 |
| | YFNKPTGYGSSSRRAPQTGIVDECCFRSCDLRRLEM | |
| | YCAPLKPAKSAGGGGSGGGGSGGGGSSEKTLRKW | |
| | LKMFKKRQLELY | |
| LR3_IGF_G4S3_ARK24 | FPAMPLSSLFVNGPRTLCGAELVDALQFVCGDRGF | 75 |
| | YFNKPTGYGSSSRRAPQTGIVDECCFRSCDLRRLEM | |
| | YCAPLKPAKSAGGGGSGGGGSGGGGSARKKAAKA | |
| | ARKKAAKAARKKAAKA | |
| LR3_IGF_G4S3_AKK24 | FPAMPLSSLFVNGPRTLCGAELVDALQFVCGDRGF | 76 |
| | YFNKPTGYGSSSRRAPQTGIVDECCFRSCDLRRLEM | |
| | YCAPLKPAKSAGGGGSGGGGSGGGGSAKKARAAK | |
| | KARAAKKARAAKKARA | |
| LR3_IGF_G4S3_AL1 | FPAMPLSSLFVNGPRTLCGAELVDALQFVCGDRGF | 77 |
| | YFNKPTGYGSSSRRAPQTGIVDECCFRSCDLRRLEM | |
| | YCAPLKPAKSAGGGGSGGGGSGGGGSRPLREKMK | |
| | PERRRPKGRGKRRREKQRPT | |
| LR3_IGF_G4S3_AL2 | FPAMPLSSLFVNGPRTLCGAELVDALQFVCGDRGF | 78 |
| | YFNKPTGYGSSSRRAPQTGIVDECCFRSCDLRRLEM | |
| | YCAPLKPAKSAGGGGSGGGGSGGGGSRRPKGRGK | |
| | RRREKQRPTDAHL | |
| LR3_IGF_G4S3_AL3 | FPAMPLSSLFVNGPRTLCGAELVDALQFVCGDRGF | 79 |
| | YFNKPTGYGSSSRRAPQTGIVDECCFRSCDLRRLEM | |
| | YCAPLKPAKSAGGGGSGGGGSGGGGSQPTRRPRPG | |
| | TGPGRRPRPRPRPTPSAPQPTRRPRPGTGPGRRPRPR | |
| | PRP | |
| LR3_IGF_G4S3_LGT25 | FPAMPLSSLFVNGPRTLCGAELVDALQFVCGDRGF | 80 |
| | YFNKPTGYGSSSRRAPQTGIVDECCFRSCDLRRLEM | |
| | YCAPLKPAKSAGGGGSGGGGSGGGGSLGTRLRAQ | |
| | SRQRSRPGRWHKVSVRW | |
| LR3_IGF_G4S3_Pep184 | FPAMPLSSLFVNGPRTLCGAELVDALQFVCGDRGF | 81 |
| | YFNKPTGYGSSSRRAPQTGIVDECCFRSCDLRRLEM | |
| | YCAPLKPAKSAGGGGSGGGGSGGGGSSPWSEWTS | |
| | SSTS | |
| LR3_IGF_G4S3_Pep186 | FPAMPLSSLFVNGPRTLCGAELVDALQFVCGDRGF | 82 |
| | YFNKPTGYGSSSRRAPQTGIVDECCFRSCDLRRLEM | |
| | YCAPLKPAKSAGGGGSGGGGSGGGGSGPWSPWDI | |
| | SSVT | |
| LR3_IGF_G4S3_Pep185 | FPAMPLSSLFVNGPRTLCGAELVDALQFVCGDRGF | 83 |
| | YFNKPTGYGSSSRRAPQTGIVDECCFRSCDLRRLEM | |
| | YCAPLKPAKSAGGGGSGGGGSGGGGSSHVVSPWSS | |
| | SSVT | |
| LR3_IGF_G4S3_Pep239 | FPAMPLSSLFVNGPRTLCGAELVDALQFVCGDRGF | 84 |
| | YFNKPTGYGSSSRRAPQTGIVDECCFRSCDLRRLEM | |
| | YCAPLKPAKSAGGGGSGGGGSGGGGSSHVVSPWSS | |
| LR3_IGF_G4S3_Pep246 | FPAMPLSSLFVNGPRTLCGAELVDALQFVCGDRGF | 85 |
| | YFNKPTGYGSSSRRAPQTGIVDECCFRSCDLRRLEM | |
| | YCAPLKPAKSAGGGGSGGGGSGGGGSWSPWSSSS | |
| | VT | |
| LR3_IGF_G4S3_ATIII | FPAMPLSSLFVNGPRTLCGAELVDALQFVCGDRGF | 86 |
| | YFNKPTGYGSSSRRAPQTGIVDECCFRSCDLRRLEM | |
| | YCAPLKPAKSAGGGGSGGGGSGGGGSAKLNSRLY | |
| | RKANKSSKLVSANRLFGDK | |
| LR3_IGF_G4S3_FibBeta | FPAMPLSSLFVNGPRTLCGAELVDALQFVCGDRGF | 87 |
| | YFNKPTGYGSSSRRAPQTGIVDECCFRSCDLRRLEM | |
| | YCAPLKPAKSAGGGGSGGGGSGGGGSQGVNDNEE | |
| | GFFSARGHRPLDKKREEAPSLRPAPPP | |

In certain embodiments, the fusion proteins comprise non-natural amino acids, including synthetic non-native amino acids, substituted amino acids, or one or more D-amino acids. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides.

In certain embodiments, the fusion proteins are retro-inverso polypeptides. A “retro-inverso polypeptide” refers to a polypeptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

In certain embodiments, the fusion proteins comprise amino acid insertions, deletions, and/or substitutions (e.g., conservative amino acid substitutions).

### III. Pharmaceutical Compositions

In one aspect, the present disclosure provides pharmaceutical compositions comprising one or more of the fusion proteins disclosed herein, and one or more pharmaceutically acceptable carriers or excipients.

The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York, incorporated, herein, by reference in its entirety).

In certain embodiments, pharmaceutical compositions according to the present disclosure are formulated to release the active agents (e.g., fusion proteins) immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the therapeutic to a particular target cell type.

Any of a number of strategies can be pursued in order to obtain controlled release in which the rate of release outweighs the rate of metabolism of the fusion proteins in question. In certain embodiments, controlled release is obtained by appropriate selection of various formulation parameters and ingredients, including, e.g., various types of controlled release compositions and coatings. Thus, the fusion protein is formulated with appropriate excipients into a pharmaceutical composition that, upon administration, releases the fusion proteins in a controlled manner. Examples include hydrogels, capsule compositions, oil solutions, suspensions, emulsions, microcapsules, molecular complexes, microspheres, nanoparticles, patches, liposomes or combinations thereof.

In certain embodiments the fusion proteins are formulated into biocompatible hydrogels. Any hydrogels that can be administered to a joint and achieve the desired release profile of a fusion proteins disclosed herein can be employed. In certain embodiments, the hydrogel comprises one or more of hyaluronic acid (HA), an HA derivative, a cellulose derivative, and a heparin-like domain polymer.

In certain embodiments, the hydrogel comprises methylcellulose. Any molecular weight of methylcellulose can be employed, e.g., between about 5 kDa and about 500 kDa. Any amount of methylcellulose can be employed in the hydrogels. In certain embodiments, the amount of methylcellulose is between about 1 and about 10% by weight of the hydrogel.

In certain embodiments, the hydrogel comprises HA (e.g, sodium hyaluronate). Any molecular weight of HA can be employed, e.g., between about 10 kDa to about 1.8 MDa. Any amount of HA can be employed in the hydrogels. In certain embodiments, the amount of HA is between about 1 and about 10% by weight of the hydrogel.

In certain embodiments, the hydrogel comprises a heparin-like domain polymer that comprises chondroitin sulfate, heparan sulfate, or heparin. Any amount of heparin-like domain polymer can be employed in the hydrogels. In certain embodiments, the amount of heparin-like domain polymer is between about 0.05% and 2% by weight of the hydrogel.

In certain embodiments, the hydrogel is thermo-setting above a certain temperature (e.g., above 35° C.). In certain embodiments, the hydrogel is fluid or shear-thinning below a certain temperature (e.g., below 35° C.).

In certain embodiments, the fusion protein is present at a concentration of between about 1 and about 1000 μg/g of a hydrogel disclosed herein. In certain embodiments, the fusion protein is present at a concentration of between about 100 and about 10,000 μg/g of a hydrogel disclosed herein. In certain embodiments, the hydrogel further comprise a glucocorticoid.

In another aspect, the present disclosure provides a composition (e.g., a pharmaceutical composition) comprising a fusion protein disclosed herein and a glucocorticoid. Suitable glucocorticoids include, without limitation, alclometasone, beclometasone, betamethasone, budesonide, chloroprednisone, ciclesonide, cortisol, cortisporin, cortivazol, deflazacort, dexamethasone, fludroxycortide, flunisolide, fluocinonide, fluocortolone, fluorometholone, fluticasone, hexacetonhydrocortamate, hydrocortisone, meprednisone, methylprednisolone, mometasone, paramethasone, prednisolone, prednisone, prednylidene, pregnadiene, pregnatriene, pregnene, proctosedyl, rimexolone, tetrahydrocorticosterone, triamcinolone and ulobetasol. Such compounds may be in the form of any and all pharmaceutically acceptable salts, hydrates and esters of such compounds including acetates (including diacetates), acetonides (including hexacetonides), furoates, phosphates and propionates (including dipropionates). In certain embodiments, the glucocorticoid is conjugated to a fatty acid (e.g., palmitic acid) via an ester bond. In certain embodiments, the glucocorticoid is contained in a microparticle carrier, such as a liposome or multilamellar vesicle. Liposomal microparticle can comprise a high melting temperature (Tₘ) lipid e.g., DSPC, DPPC or HSPC. In certain embodiments, the glucocorticoid is contained in a liposomal microparticle and is present at between 0.1-20 molar percent of the liposome lipid. In certain embodiments, glucocorticoid is contained in a liposomal microparticle and the liposome lipid is between 0.01%-10% by weight of the hydrogel. In certain embodiments, the glucocorticoid is present in the hydrogel at a concentration sufficient to stimulate cartilage matrix synthesis or stimulate cell survival or prevent cartilage matrix degradation or prevent cell death when the pharmaceutical composition (e.g., a hydrogel) is injected into a joint. In certain embodiments, the glucocorticoid is present at a concentration between 1-1000 μg/g of hydrogel.

In certain embodiments, after injection of the composition into the intra-articular space (e.g., synovial fluid) of a joint, the cartilage matrix synthesis or degradation readouts of the joint show improvement over the readouts after injection of the fusion protein or the combination of the fusion protein plus glucocorticoid alone.

In certain embodiments, after injection of the composition into the intra-articular space (e.g., synovial fluid) of a joint, the glucocorticoid is present in the joint with a half-life of at least about 8 days (e.g., 9, 10, 11, or 12 days).

In certain embodiments, after injection of the composition into the intra-articular space (e.g., synovial fluid) of a joint, the fusion protein is retained in the intra-articular space of the joint for a longer time than either the fusion protein or glucocorticoid when injected alone.

### IV. Methods of Treatment

In one aspect, the present disclosure provides methods of treating musculoskeletal condition (e.g., osteoarthritis) by administering the fusion proteins and pharmaceutical compositions disclosed herein to a subject.

In certain embodiments, the present disclosure provides a method of treatment of a musculoskeletal condition, comprising administrating a therapeutically effective amount of a fusion protein or pharmaceutical composition thereof disclosed herein into a joint cavity of a subject. Suitable musculoskeletal conditions include, without limitation, osteoarthritis, one or more cartilage defects, rheumatoid arthritis, post-injury cartilage degradation, acute inflammatory arthritis, infectious arthritis, osteoporosis, or side-effects from other pharmacologic interventions.

In certain embodiments, the methods of treatment described herein, further comprise selection of such a subject suffering from a musculoskeletal condition. Such selection is performed by the skilled practitioner by a number of available methods, for instance, assessment of symptoms which are described herein.

Successful treatment is evidenced by amelioration of one or more symptoms of the musculoskeletal condition. Administering a fusion protein disclosed herein to subject in need thereof is expected to prevent or retard the development of the musculoskeletal disease. The term “prevention” is used to refer to a situation wherein a subject does not yet have the specific condition being prevented, meaning that it has not manifested in any appreciable form. Prevention encompasses prevention or slowing of onset and/or severity of a symptom, (including where the subject already has one or more symptoms of another condition). Prevention is performed generally in a subject who is at risk for development of a condition or physical dysfunction. Such subjects are said to be in need of prevention.

In certain embodiments, the methods of prevention described herein, further comprise selection of such a subject at risk for a musculoskeletal condition, prior to administering a fusion protein to the subject, to thereby prevent the musculoskeletal condition. Such selection is performed by the skilled practitioner by a number of available methods. For instance, assessment of risk factors or diagnosis of a disease which is known to cause the condition or dysfunction, or treatment or therapy known to cause the condition. Subjects which have a disease or injury or a relevant family history which is known to contribute to the condition are generally considered to be at increased risk.

As used herein, the terms “treat” or “treatment” or “treating” refers to both therapeutic treatment and prophylactic (i.e. preventative) measures, wherein the object is to prevent or slow the development of the disease, such as reducing at least one effect or symptom of a musculoskeletal condition. Treatment is generally “effective” if one or more symptoms or clinical markers are reduced as that term is defined herein. Alternatively, treatment is “effective” if the progression of a musculoskeletal condition is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “effective amount” as used herein refers to the amount of a pharmaceutical composition comprising one or more fusion proteins disclosed herein, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylactically significant reduction in a symptom or clinical marker associated with a musculoskeletal condition.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated.

With reference to the treatment of a subject with a musculoskeletal condition, the term “therapeutically effective amount” refers to the amount that is safe and sufficient to prevent or delay the development and further decrease the musculoskeletal condition in patients. The amount can thus cure or cause a decrease in at least one symptom of the musculoskeletal condition. The effective amount for the treatment of a disease depends on the type of disease, the species being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of musculoskeletal disease. When using an experimental animal model, efficacy of treatment is evidenced when a reduction in a symptom of musculoskeletal disease is shown versus untreated animals.

As used herein, the terms “administering,” and “introducing” are used interchangeably herein and refer to the placement of the therapeutic agents such as one or more fusion proteins to a subject by a method or route which results in delivering of such agent(s) at a desired site. The fusion proteins can be administered by any appropriate route which results in an effective treatment in the subject.

The one or more fusion proteins or compositions thereof as disclosed herein may be administered by any route known in the art or described herein, for example, oral, parenteral (e.g., intravenously or intramuscularly), intra-peritoneal, rectal, cutaneous, nasal, vaginal, inhalant, skin (patch), or ocular. In certain embodiment, the fusion proteins or compositions thereof are administered by direct intra-articular injection. The fusion proteins or compositions disclosed herein may be administered in any dose or dosing regimen.

The fusion proteins or compositions may be administered to the patient in a single dose or in multiple doses. When multiple doses are administered, the doses may be separated from one another by, for example, one hour, three hours, six hours, eight hours, one day, two days, one week, two weeks, or one month. For example, the fusion proteins or compositions disclosed herein may be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the fusion proteins or compositions disclosed herein can be increased if the lower dose does not provide sufficient therapeutic activity.

While the attending physician ultimately will decide the appropriate amount and dosage regimen, therapeutically effective amounts of the fusion proteins may be provided at a dose of 0.0001, 0.01, 0.01 0.1, 1, 5, 10, 25, 50, 100, 500, or 1,000 mg/kg. Effective doses may be extrapolated from dose-response curves derived from in vitro or animal model test bioassays or systems.

Dosages for a particular patient or subject can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or half-life of the fusion proteins and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions are optionally tested in one or more appropriate in vitro and/or in vivo animal models of disease, such as models of musculoskeletal disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the fusion proteins. Administration can be accomplished via single or divided doses.

In determining the effective amount of the fusion proteins to be administered in the treatment or prophylaxis of disease the physician evaluates circulating plasma levels, formulation toxicities, and progression of the disease.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred.

Actual dosage levels of the active ingredients in the pharmaceutical compositions of this disclosure may be varied so as to obtain an amount of the active ingredient which is effective to achieve the desired therapeutic response for a particular subject, composition, and mode of administration, without being toxic to the subject.

The selected dosage level will depend upon a variety of factors including the activity of the particular fusion protein employed, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

### V. Other Embodiments

From the foregoing description, it will be apparent that variations and modifications may be made to the invention described herein to adopt it to various usages and conditions. Such embodiments are also within the scope of the following claims.

The disclosure also contemplates an article of manufacture which is a labeled container for providing the fusion proteins disclosed herein. An article of manufacture comprises packaging material and a pharmaceutical agent of the fusion proteins disclosed herein contained within the packaging material.

The pharmaceutical agent in an article of manufacture is any composition suitable for providing the fusion proteins disclosed herein. Thus, the composition can comprise the one or more polypepetides as disclosed herein or a mutant, or derivative thereof or a DNA molecule which is capable of expressing such a peptide.

The article of manufacture contains an amount of pharmaceutical agent sufficient for use in treating a condition indicated herein, either in unit or multiple dosages. The packaging material comprises a label which indicates the use of the pharmaceutical agent contained therein.

The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain the pharmaceutical agent.

In preferred embodiments, the packaging material includes a label that is a tangible expression describing the contents of the article of manufacture and the use of the pharmaceutical agent contained therein.

### VI. Examples

The following examples should not be construed as limiting the scope of this disclosure.

### Example 1

### Methods for Protein Expression and Purification

Methods for producing proteins transiently in 293F cells Nucleic acids encoding the desired protein sequence are cloned into pCep4 vector (Invitrogen) using standard recombinant DNA techniques. Cloned vectors are amplified by growing transformed NEB 5-alpha Competent E. coli (New England Biolabs) in 1 L Luria Broth with ampicillin selection overnight at 37° C. shaking at 2000 rpm. Cells are harvested by spinning at 5000 g for 20 minutes and vector DNA is extracted from the bacterial pellet using QIAfilter® Plasmid Mega Kit (Qiagen). 293F cell culture media is prepared by adding 20 mL of 200 mM L-Glutamine (source?) and 10 mL of 10% Pluronic F-68 to 1 L of F17 media (Invitrogen®). For transient transfections, 1 L of 293F cells is grown to a density of 1.5-2.0 million/mL at 37° C. and 5% CO₂. One mg of total protein and 2.5 mL of polyethileneimine solution (1 mg/mL) are mixed in 50 mL of cell culture media, vortexed, and added to the cells after 15 minutes of incubation. Transfected cells are fed at 24 and 72 hours post transfection with peptone (20% w/v stock solution in F17 medium sterilized through 0.22 μm filter) to a final concentration of 0.5%. After cell viability drops to below 80% (generally one week), cells are harvested by centrifugation at 4000 g for 20 minutes. Resultant supernatant is filtered through a 0.22 μm filter.

Methods for Producing Proteins from Stably Transfected CHO Pools

Nucleic acids encoding the desired protein sequence synthesized at DNA 2.0 are cloned into pMP10K (an in-house proprietary vector) using standard recombinant DNA techniques. Cloned vectors are amplified in 10 mL Luria broth with ampicillin selection overnight at 37° C. shaking at 2000 rpm. Vector DNA is extracted from bacteria using QIAprep® Spin Miniprep Kit (Qiagen). Suspension adapted CHO K1 cells are grown in EX-CELL® CD CHO Serum-Free Medium (Sigma-Aldrich®) in a baffled shake flask to a density of no more than 2 million/mL at 37° C., 5% CO₂. On the day of transfection, cells are resuspended in Opti-MEM® I Serum-Free Medium (Invitrogen®) to a density of 80,000 cells/mL and 500 μL of cells is distributed in a 24-well tissue culture plate (one well per transfection). The cells are then transfected with 1 μg total DNA, including 10 ng of selectable pNeo vector (carrying the neomycin selection marker) using 2.75 μL of Lipofectamine® LTX (Life Technologies™). Three hours post-transfection, 1 mL of HAMS-F12 (Invitrogen®) media supplemented with 10% FBS is added and the cells are allowed to recover in a 37° C., 5% CO₂ incubator for 48 hours. Cell media is then replaced with HAMS-F12 plus Geneticin® at 0.5 mg/mL and the cells are incubated for four days under selection. Media is replaced with EX-CELL® CD CHO, plus Geneticin®, and cells are incubated for 2 to 3 weeks until colonies form and all untransfected cells have died off. Selected transfected cells are then expanded into 25 mL flask until there are enough cells to seed a 125 mL shake flask with 25 mL of 0.3×10⁶ cells/mL. Expansion of cells is continued with seedings at 0.3×10⁶ cells/mL until desired volume is reached. When cell density reaches over 5×10⁶ cells/ml, Hyclone® Cell Boost 5 (Thermo Scientific) is added at 10% total volume. Cells are harvested by centrifugation at 6000 g when viability drops below 60%. Supernatant is filtered through AcroPak™ 1000 0.8/0.2 μm Capsules (Pall Corporation).

### Protein Purification on Nickel Charged Resin

6×-Histidine-tagged proteins are purified using AKTA™ FPLC™ (GE Healthcare Life Sciences). 5 mM imidazole and 500 mM of NaCl is added to filtered supernatant containing protein to be purified. A freshly packed 25 mL column (1.6 cm inner diameter) of Ni-NTA Superflow (Qiagen®) nickel charged resin is equilibrated with the running buffer provided (PBS, plus 0.5 M NaCl, pH 7.4). Supernatant is loaded onto the column at 10 mL/minute. The column is washed with 6× column volumes of PBS, 500 mM NaCl. Bound protein is eluted from column with 300 mM imidazole. Fractions containing protein are pulled and dialyzed overnight in PBS.

### Protein Purification of IGF(LR3)-PRELP

IGF(LR3)-PRELP is purified from 0.2 μM filtered supernatant using two chromatography steps: cation exchange and anion exchange. In the first chromatography step, a SPFF™ cation exchange column (inner diameter 1.6 cm, bed height 10 cm, GE Healthcare Sciences) is equilibrated with 0.5×PBS (pH 7.4). The filtered supernatant is diluted 1:1 with distilled water, and loaded on the cation exchange column The bound material is washed with 0.5×PBS (pH 7.4), and eluted using a step gradient (1×PBS+500 mM NaCl, pH 7.4). All chromatography steps are performed at a flowrate of 500 cm/hr. Eluted fractions containing protein are pooled, diluted with distilled water to a conductance of 10 mS/cm, and pH adjusted with 1M Tris Base to 8.0.

In the second chromatography step, a QSFF™ anion exchange column (inner diameter 1.6 cm, bed height 10 cm, GE Healthcare Sciences) is equilibrated with 0.5×PBS (pH 8.0). The cation exchange pool (pH and conductance adjusted) is loaded on the anion exchange column at a flowrate of 300 cm/hr. The flow through is collected, concentrated and dialyzed against 2×PBS (pH 7.4).

### SDS-PAGE Analysis

Proteins are run on a 4-12% SDS-PAGE gel in reduced and non-reduced conditions and visualized by staining with SimplyBlue™ SafeStain (Invitrogen®). Gels are microwaved in water for one minute and then in stain for two minutes to expedite the staining process. Gels are destained by microwaving in water for two minutes. This method is used to determine whether the purified protein is the correct size and if it is pure.

### Size Exclusion Chromatography

Size Exclusion Chromatography (SEC) is used to assess the purity and monomeric state of a protein. 50 μg of protein is injected on a TSKgel® SuperSW3000 column (4.6 mm ID×30 cm) (Tosoh Bioscience) in 10 mM phosphate buffer with 450 mM NaCl at 0.35 mL/minute. All measurements are performed on an Agilent 1100 HPLC, equipped with an auto sampler, a binary pump and a diode array detector. Data is analyzed using ChemStation® software (Agilent Technologies).

### Example 2

### Binding of Fusion Proteins to Heparan Sulfate and Chondroitin Sulfate

The specificity of binding of fusion proteins to the polysaccharides heparan sulfate and chondroitin sulfate may be determined by measuring the ability of the proteins to bind polysaccharides coated on an ELISA plate. Heparin Binding Plates (BD Biosciences) are coated with 50 μl of 2-10 μg/mL concentration of heparan sulfate or chondroitin sulfate (Sigma-Aldrich®) and incubated overnight at room temperature. Plates are washed with PBS and blocked with 250 μl of 0.2% gelatin in PBS for 1 hour at 37 C. Plates are then washed with PBS and tapped dry. 50 μl of protein in a dilution series is added to the wells and incubated for 2 hours at 37 C. The protein dilution series starts from 100 nM and includes ten additional three-fold dilutions in PBS, 0.2% gelatin and one blank (PBS, 0.2% gelatin only). After the plates are washed in PBS, 50 μl of anti-human IGF-1 (Abcam) at 1:250 in PBST is added and the plates are incubated for 1 hour rotating at room temperature. Plates are washed with PBST and 100 μl of 1:1000 anti-Rabbit-HRP (Cell Signaling Technology®) in PBST is added to each well and the plate is incubated for 1 hour at room temperature. Plates are then washed with PBST and incubated with 100 μl TMB substrate for 5-10 minutes at room temperature and the reaction is stopped by adding 100 μl Stop solution. The absorbance is measured at 450 nm and the resulting data is analyzed using GraphPad Prism® (GraphPad Software, San Diego, Calif.).

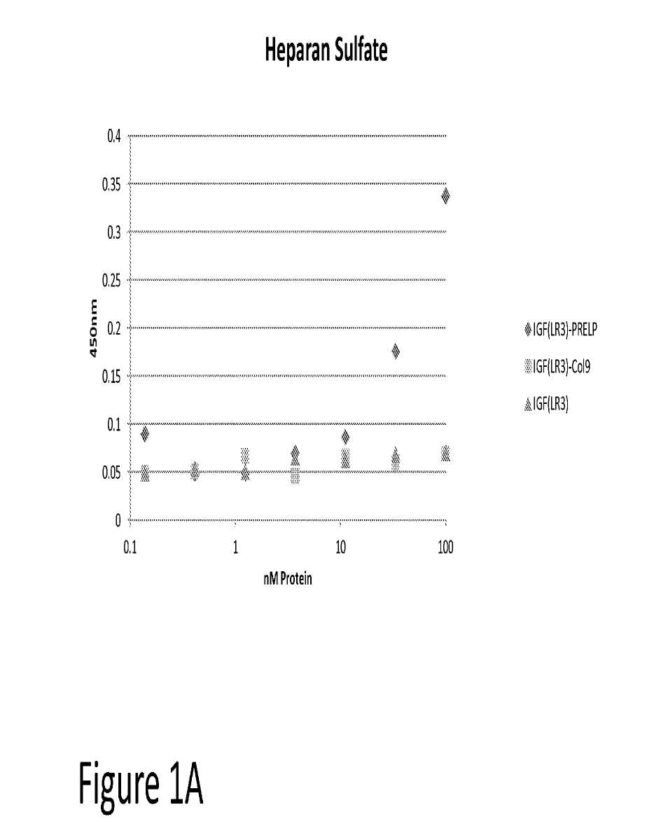

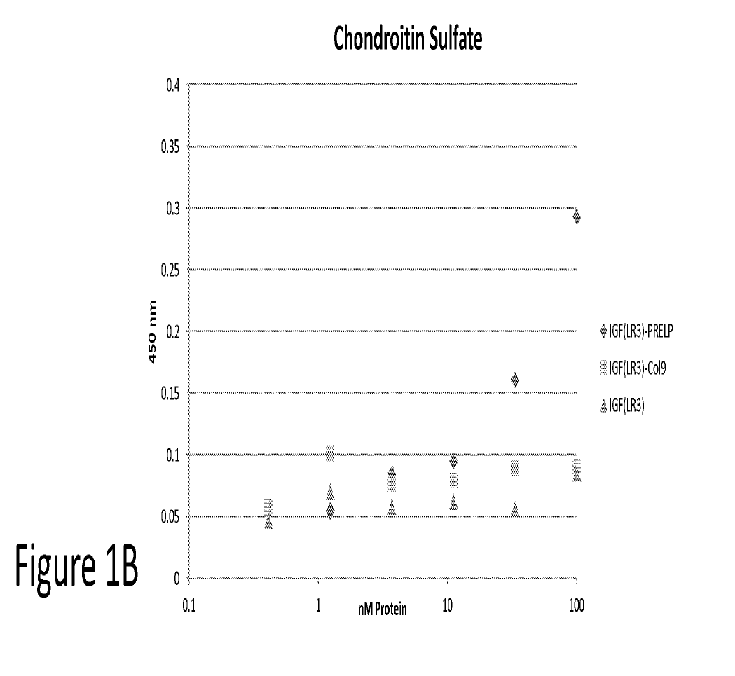

The method above was used to determine the specificity of two fusion proteins, GF-Fus3 (6×-Histidine-tagged) and GF-Fus4 (6×-Histidine-tagged). GF-Fus1 (6×-Histidine-tagged) with no fused binding domain, was used as a negative control. As shown in FIG. 1, GF-Fus3 bound to plates coated with heparan sulfate (FIG. 1A) and chondroitin sulfate (FIG. 1B), whereas GF-Fus4 did not bind.

### Example 3

### Binding of Fusion Proteins to Collagen II

Specificity of proteins to collagen type II may be determined by measuring the ability of the protein to bind the collagen coated on an ELISA plate. Reacti-Bind® 96 well plates are coated overnight at 4° C. with 100 μl of collagen type II (Chondrex Products, Redmond, Wash.) with 1× supplied buffer. Plates are washed with PBS, 0.05% Tween-20 (PBST) and blocked for 1 hour at room temperature with 100 μl of Protein-Free Blocking Buffer (Pierce, Thermo Scientific). Plates are then washed with PBST and tapped dry. 50 μL of protein in a dilution series is added to the wells and incubated for 1 hour at room temperature. The protein dilution series starts from 100 μM and includes ten additional three-fold dilutions in PBS and one blank (PBS). After plates are washed in PBS, 50 μL of anti-human IGF-1 (Abcam) at 1:250 in PBST is added and plates are incubated for 1 hour rotating at room temperature. The plates are then washed with PBST and 100 μL of 1:1000 anti-Rabbit-HRP (Cell Signaling Technology) in PBST for 1 hour at room temperature. Plates are washed with PBST and incubated with 100 μL TMB substrate for 5-10 minutes at room temperature and the reaction is stopped by adding 100 μL Stop Solution. The absorbance is measured at 450 nm and the resulting data is analyzed using GraphPad Prism®.

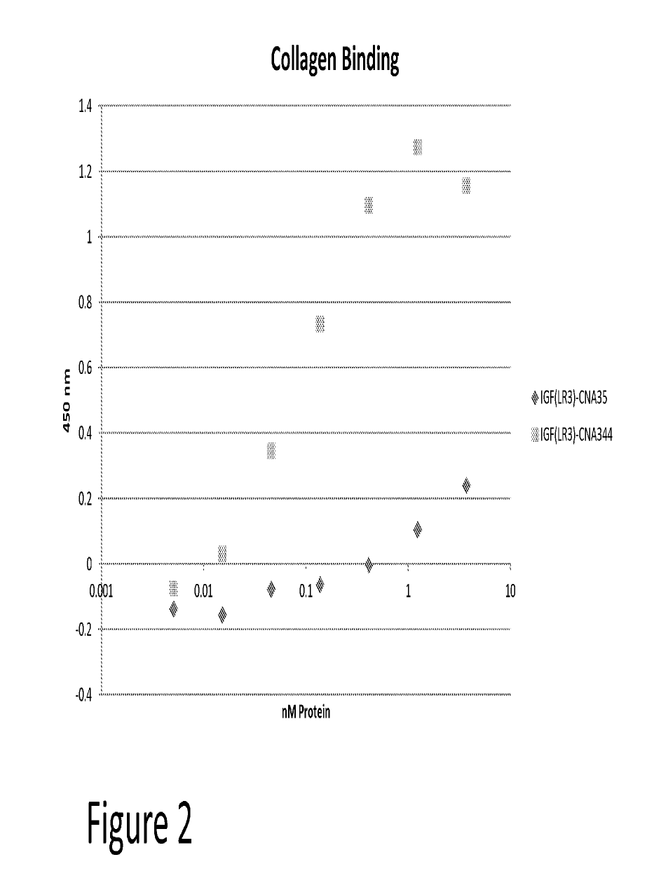

The binding of two anticipated type II collagen binding fusion proteins, GF-Fus5 (6×-Histidine-tagged) and GF-Fus6 (6×-Histidine-tagged) was measured as described above. As shown in FIG. 2, both GF-Fus5 and GF-Fus6 bound to collagen type II, with GF-Fus6 binding more strongly.

### Example 4

### Stimulation of AKT Phosphorylation by Fusion-Protein-Stimulated Primary Bovine Chondrocytes in High Density Culture

Fusion proteins comprising IGF-1 and a cartilage matrix binding domain were prepared as described above. Six fusion proteins were prepared: GF-Fus1 (6×-Histidine-tagged), GF-Fus2(6×-Histidine-tagged), GF-Fus3 (6×-Histidine-tagged), GF-Fus4 (6×-Histidine-tagged), GF-Fus5 (6×-Histidine-tagged), and GF-Fus6 (6×-Histidine-tagged). In order to ensure that the growth factor portion of the fusion protein was active in the fusion proteins, each construct was tested for its ability to stimulate AKT phosphorylation. Wild-type IGF-1 (wtIGF) was included as a control. Bovine chondrocytes were stimulated with a range of doses of each fusion protein, and pAKT levels were measured by ELISA.

### Chondrocyte Isolation and Ligand Stimulation

Bovine chondrocytes are isolated from the femoral chondyles of 2-4 week old bovine calves. Knee joints are mounted by removing all tissue surrounding the femur, removing the femoral head with a bone saw or hack saw, and clamping in a tissue vice. Joints are aseptically opened, removing the patella, tibia and fibula. Using a scalpel, cartilage is sliced off the femoral chondyles and placed in sterile PBS (pH=7.4) containing penicillin-streptomycin (1×, Gibco 15140-122). PBS is subsequently removed and pronase solution (50 mL per 5 g of tissue), consisting of high glucose DMEM (Life Technologies Cat#11965-092), fetal bovine serum (10% v/v, Life Technologies Cat#16140071), HEPES (100 mM, Gibco 15630-080), non-essential amino acids (1×, Sigma M7145), penicillin-streptomycin (1×, Gibco 15140-122), proline (400 μM, Sigma P5607-256), Protease Type XIV (2 mg/mL, Sigma Cat# P5147), is added for 1 hour with stirring. After rinsing twice with sterile PBS (pH=7.4), collagenase solution (50 mL per 5 g of tissue), consisting of high glucose DMEM (Life Technologies Cat#11965-092), fetal bovine serum (10% v/v, Life Technologies Cat#16140071), HEPES (100 mM, Gibco 15630-080), NEAA (1×, Sigma M7145), Penicillin-Streptomycin (1×, Gibco 15140-122), 0.25 mg/mL Collagenase P (Roche Cat#11 249 002 001), is added for 18 hours with stirring. Cell are strained, washed, and resuspended in chondrocyte culture medium (low glucose DMEM (1× Gibco 11885-084), Penicillin-Streptomycin (1×, Gibco 15140-122), non-essential amino acids (1×, Sigma M7145), and HEPES (100 mM, Gibco 15630-080)) with fetal bovine serum (10% v/v, Life Technologies Cat#16140071).

For ligand stimulation, chondrocytes were seeded at 200,000 cells/well into 96-well tissue culture plates with 100 μL of chondrocyte culture medium with fetal bovine serum (10% v/v, Life Technologies Cat#16140071). Alternatively, BXPC-3 cells, a pancreatic adenocarcinoma cell line (ATCC® CRL-1687™), were seeded at 30,000 cells/well into 96-well tissue culture plates with 100 μL of BXPC-3 medium (RPMI-1640, e.g., ATCC® 30-2001™), with L-glutamine (2 mM), Penicillin-Streptomycin (1×, Gibco® 15140-122), Fetal Bovine Serum (10% v/v, Life Technologies Cat#16140071)). After 24 hrs, both chondrocytes and BXPC-3 cells were rinsed with 100 μL of sterile PBS (pH=7.4) per well and 100 μl of chondrocyte or BXPC-3 culture medium (without fetal bovine serum) was added to the corresponding cell type. After an additional 24 hrs, cells were stimulated with the doses of fusion proteins set forth in Table 4 by adding 25 μL of fusion protein at a concentration 5 times greater than the concentration listed in Table 4 to the 100 μL of medium already in the wells. After 10 min of stimulation, fusion protein was removed and wells were rinsed in ice cold PBS (pH=7.4). 50 μL/well of cell extraction buffer (Invitrogen cat# FNN0011) was added and incubated with shaking at 4° C. for 30 minutes. Lysates were then frozen at −80° C.

TABLE 4
Ligand doses for chondrocyte stimulation with wtIGF, GF-Fus1, GF-Fus2,
GF-Fus3, GF-Fus4, GF-Fus5, and GF-Fus6

| | Final 1X Concentration (M) | | | | | | | | | |
|---|---|---|---|---|---|---|---|---|---|---|
| Dose | 2.67E−07 | 6.67E−08 | 1.67E−08 | 4.17E−09 | 1.04E−09 | 2.6E−10 | 6.51E−11 | 1.63E−11 | 4.07E−12 | 0 |

Quantification of pAKT by ELISA

Corning high binding 384 well black ELISA plates (cat#3577) are coated with 30 μL of capture antibody at 4 μg/mL (anti-AKT1 total capture antibody, Upstate, Cat#05-591MG) in PBS (pH=7.4) for 16 hours at room temperature, washed and blocked with 50 μl per well 2% bovine serum albumin (Sigma Cat# A3294) in PBS (pH=7.4) for 1 hour at room temperature. 20 μL of thawed lysates or recombinant pAKT standards (ten 2-fold serial dilutions of 400 ng recombinant human AKT, active (Upstate, Cat#14-276) in PBS (pH=7.4) containing 50% v/v Cell extraction buffer (Invitrogen Cat#FNN0011), 1% bovine serum albumin (Sigma Cat# A3294), 0.05% Tween20) are applied to the coated plate and incubated for 2 hours at room temperature. After washing 3 times with 100 μL/well of 0.05% Tween-20/PBS (pH=7.4) bound phospho-AKT is detected using Phospho-AKT (Ser473)(587F11)-biotinylated (Cell Signaling, Cat#5102), streptavidin-HRP (R&D Systems Cat# DY998, Part #890803), SuperSignal ELISA PicoChemiluminescent Substrate (Thermo Scientific Cat#37069), using a luminometer to detect light emissions at 425 nm.

### Results

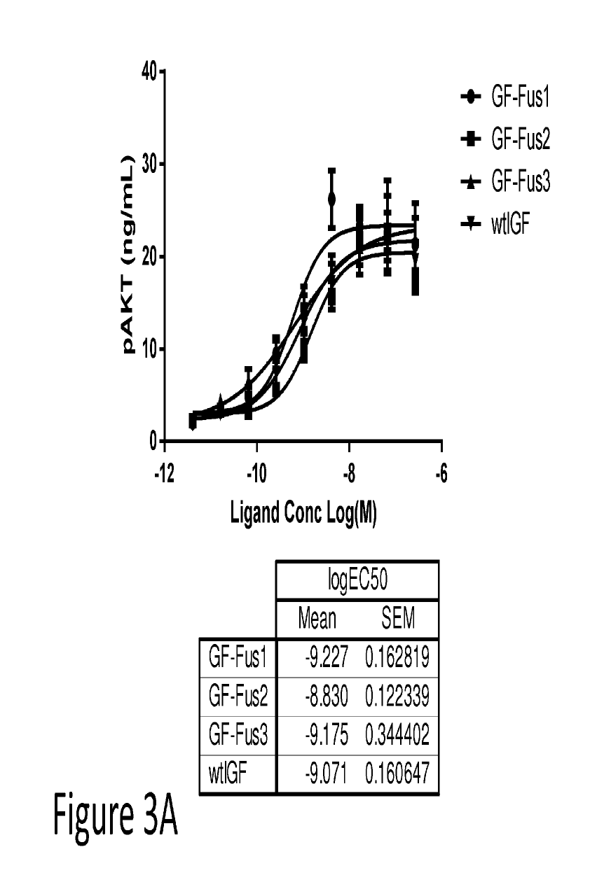

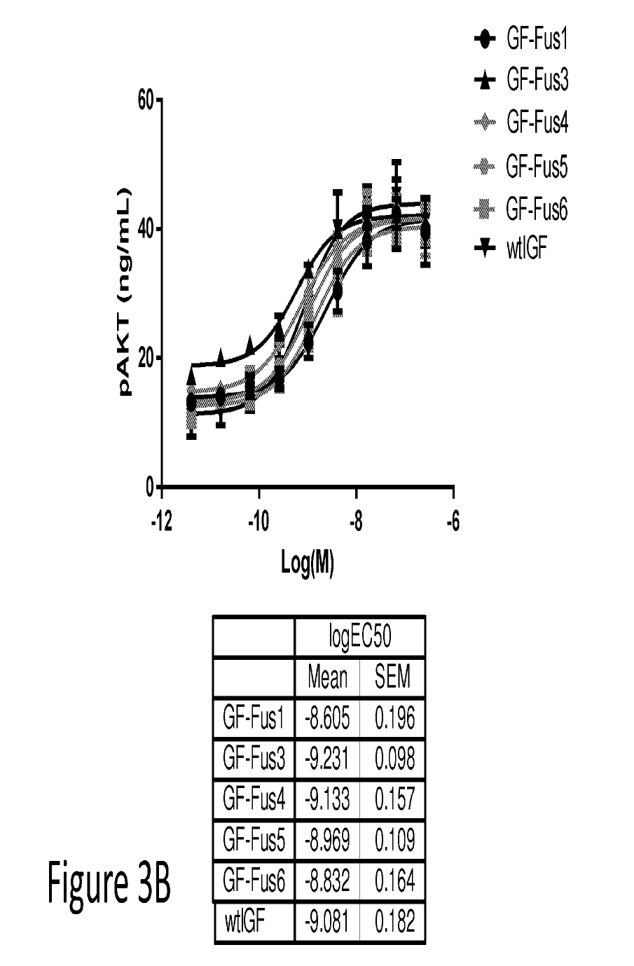

As shown in FIG. 3A, GF-Fus1, GF-Fus2, and GF-Fus3 stimulated phosphorylation of AKT to a similar extent as wild-type IGF. The obtained EC₅₀ values (shown in Table 5A) demonstrate that the fusion proteins are functionally equivalent to wild-type IGF in this assay. In FIG. 3B, GF-Fus1, GF-Fus3, GF-Fus4, GF-Fus5, and GF-Fus6 stimulated phosphorylation of AKT to a similar extent as wild-type IGF. The obtained EC₅₀ values (shown in Table 5B) demonstrate that the fusion proteins are functionally equivalent to wild-type IGF in this assay.

TABLE 5A
EC₅₀ of fusion proteins

| | logEC₅₀ | |
|---|---|---|
| | Mean | SEM |
| GF-Fus1 | −9.227 | 0.162819 |
| GF-Fus2 | −8.830 | 0.122339 |
| GF-Fus3 | −9.175 | 0.344402 |
| wtIGF | −9.071 | 0.160647 |

TABLE 5B
EC₅₀ of fusion protein stimulation of BXPC-3 cells

| | logEC₅₀ | |
|---|---|---|
| | Mean | SEM |
| GF-Fus1 | −8.605 | 0.196 |
| GF-Fus3 | −9.231 | 0.098 |
| GF-Fus4 | −9.133 | 0.157 |
| GF-Fus5 | −8.969 | 0.109 |
| GF-Fus6 | −8.832 | 0.164 |
| wtIGF | −9.081 | 0.182 |

### Example 5

### Sustained Activity of GF-Fus3 and GF-Fus 2 in an In Vitro Joint Disease Model Washout Experiment Using Explanted Bovine Cartilage

The activity of both the matrix binding arm and the growth factor arm of fusion proteins containing the heparin binding domain from PRELP were assessed in an in vitro joint disease model washout experiment using explanted bovine cartilage as described below.

### Methods

Bovine cartilage explants (3 mm diameter, 1.2 mm thick) are harvested from the femoral patellar groove of 2-4 week old bovine calves. Knee joints are mounted by removing all tissue surrounding the femur, removing the femoral head with a bone saw or hack saw, and clamping in a tissue vice. Joints are aseptically opened, removing the patella, tibia and fibula. Using a 3 mm diameter disposable biopsy punch, approximately 80×3 mm diameter full thickness cartilage cores are punched from the femoral-patellar groove. A sterile knife is used to slice the cores at the cartilage-bone interface. Cores are then inserted into 3 mm diameter holes in a 1.2 mm thick sterile stainless steel plate and sliced flush with the plate using sterile razor blades to remove the excess core length, resulting in 3 mm diameter, 1.2 mm thick cartilage explants with the superficial zone cartilage intact.

Explants are cultured in 96-well plates in 300 μL of medium consisting of low glucose DMEM (1× Gibco 11885-084), Penicillin-Streptomycin (1×, Gibco 15140-122), ascorbic acid (20 μg/mL, Sigma A4403), proline (400 μM, Sigma P5607-256), non-essential amino acids (1×, Sigma M7145), and HEPES (100 mM, Gibco 15630-080). Explants from three animals (n=2-3 per animal) are used in each treatment condition for a total explant number of 8-9 per condition. Medium is changed every 2 days. On days 6 and 10, the medium is supplemented with 5 μCi/mL of ³⁵S-sodium sulfate (Perkin Elmer NEX041H001MC, 1 mCi).

Timepoints are taken at day 8 and 12: explant tissue is washed four times for 30 min each (2 hr total) in 1 mM unlabeled sodium sulfate in PBS. Each explant is weighed wet and frozen at −20° C. until digestion. Tissue digestion was performed with Proteinase K (Roche cat #3115879001) and each explant is digested in 1 mL 100 μg/mL Proteinase K in 50 mM Tris-HCL, 1 mM Calcium Chloride pH=8.0 buffer at 60° C. overnight. Measurement of sGAG content and DNA content is performed using standard assay methods such as those described in Hoemann, 2004, Methods in Molecular Medicine: Cartilage and Osteoarthritis. Totowa, N.J.: Humana Press Inc.; p. 127-52. ³⁵S-sulfate content of the digested cartilage explants was quantified by mixing 20 μL of digest with 250 μL of scintillation fluid (Perkin Elmer cat #1200-439) and counting with a WALLAC 1450 MICROBETA TRILUX scintillation counter.

The experimental design is summarized in Table 6 herein. Treatments were given at the following concentrations: wtIGF-1 13.3 nM=100 ng/mL (IGF-1 R&D systems 291-G1); GF-Fus1 (6×-Histidine tagged) 13.3 nM=135 ng/mL; GF-Fus2 (6×-Histidine tagged) 13.3 nM=140 ng/mL; and GF-Fus3 (6×-Histidine tagged) 13.3 nM=181 ng/mL. All treatments included IL-1 and were given for either 4 days or the entire culture duration. The outcomes measured were ³⁵S-sulfate incorporation, DNA content, and sGAG content of plug at endpoint, and sGAG released to media for all media changes. Controls were either no treatment (Healthy) or IL-1 alone at 1 ng/mL (Disease).

TABLE 6
Experimental design

| | Time point | | | | | | # explants |
|---|---|---|---|---|---|---|---|
| Control | (Day) | IL-1α | wtIGF | GF-Fus2 | GF-Fus1 | GF-Fus3 | (n) |
| Healthy | 8 | − | − | − | − | − | 9 |
| Disease | 8 | + | − | − | − | − | 9 |
| − | 8 | + | +4 days | − | − | − | 9 |
| − | 8 | + | +8 days | − | − | − | 9 |
| − | 8 | + | − | +4 days | − | − | 9 |
| − | 8 | + | − | +8 days | − | − | 9 |
| − | 8 | + | − | − | +4 days | − | 9 |
| − | 8 | + | − | − | +8 days | − | 9 |
| − | 8 | + | − | − | − | +4 days | 9 |
| − | 8 | + | − | − | − | +8 days | 9 |
| Healthy | 12 | − | − | − | − | − | 9 |
| Disease | 12 | + | − | − | − | − | 9 |
| − | 12 | + | +4 days | − | − | − | 8 |
| − | 12 | + | +12 days | − | − | − | 9 |
| − | 12 | + | − | +4 days | − | − | 9 |
| − | 12 | + | − | +12 days | − | − | 9 |
| − | 12 | + | − | − | +4 days | − | 9 |
| − | 12 | + | − | − | +12 days | − | 9 |
| − | 12 | + | − | − | − | +4 days | 9 |
| − | 12 | + | − | − | − | +12 days | 9 |

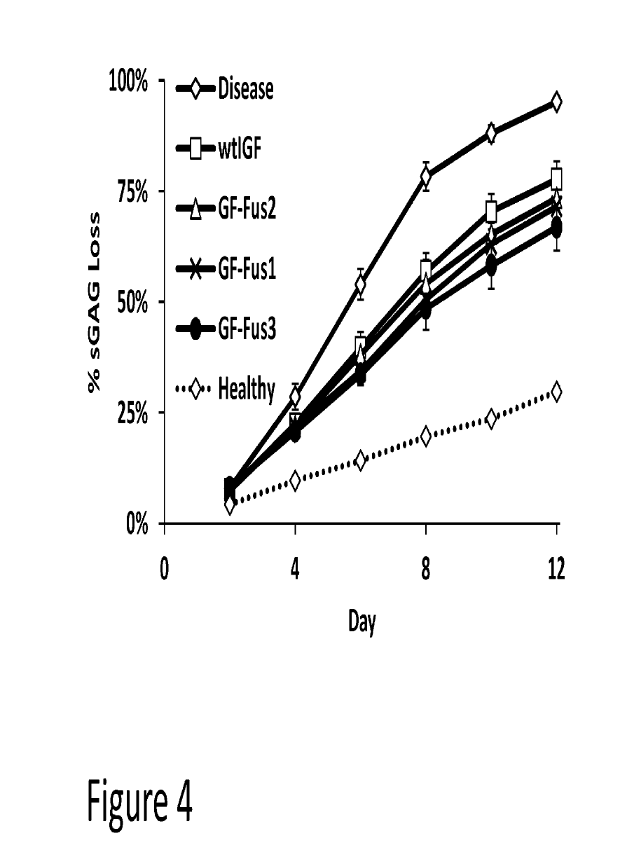

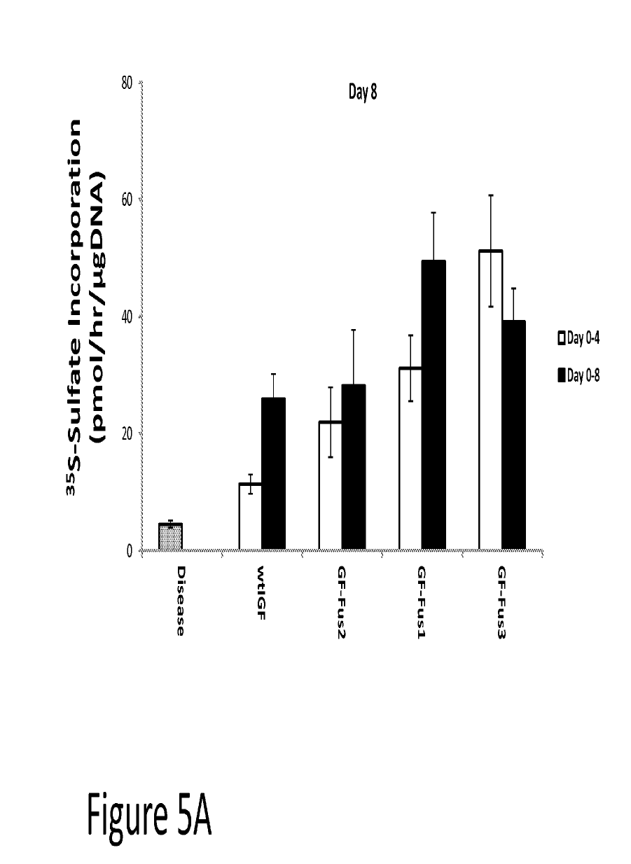

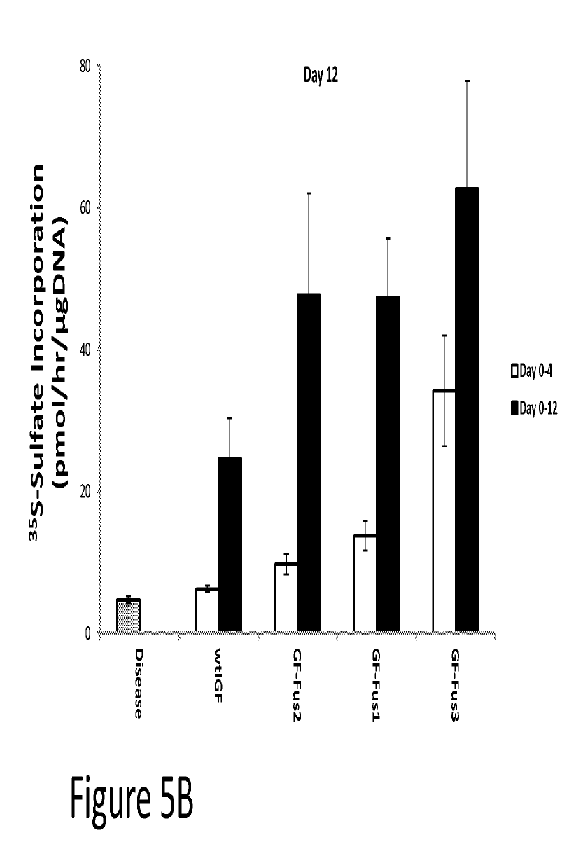

FIG. 4 depicts a graph of cartilage matrix loss as measured by the cumulative percentage of total sGAG lost to the culture medium against time (days). Percentage loss is calculated by dividing the cumulative sGAG in the culture medium by the total sGAG present in the medium over the culture period plus the sGAG remaining in the explant at the end of culture. Cartilage matrix loss was reduced by each IGF fusion protein relative to the disease control at a level similar to wild-type IGF. New cartilage matrix synthesis (i.e. sulfated proteoglycan synthesis is measured by ³⁵S-Sulfate incorporation) during the final 48 hrs of cultures terminated at day 8 and day 12 is shown in FIGS. 5A and 5B, respectively. Cartilage matrix synthesis was increased compared to disease control by all fusion proteins (GF-Fus1, GF-Fus2, and GF-Fus3) and wild-type IGF when the fusion proteins were supplied in every medium change for the entire culture duration of 8 and 12 days (black bars). However, stimulation of cartilage matrix synthesis 4 or 8 days after fusion protein removal was highest for the PRELP heparin binding domain fusion to LR3-IGF (GF-Fus3, FIGS. 5A and 5B).

### Example 6

### Activity of Collagen Binding Growth Factors in an In Vitro Joint Disease Model Washout Experiment Using Explanted Bovine Cartilage

Fusion proteins that bind to type II collagen (GF-Fus5, GF-Fus6) were prepared (both were 6×-Histidine tagged). The fusion proteins were characterized using the methods and outcome measures described above in Example 5, modified by treatments summarized in Table 7, herein. All conditions used explants harvested from a single animal.

TABLE 7
Experimental design

| | Time point | | | | | | # explants |
|---|---|---|---|---|---|---|---|
| Control | (Day) | IL1α | GF-Fus1 | GF-Fus3 | GF-Fus5 | GF-Fus6 | (n) |
| Healthy | 8 | − | − | − | − | − | 6 |
| Disease | 8 | + | − | − | − | − | 6 |
| − | 8 | + | +4 days | − | − | − | 6 |
| − | 8 | + | +8 days | − | − | − | 6 |
| − | 8 | + | − | +4 days | − | − | 6 |
| − | 8 | + | − | +8 days | − | − | 6 |
| − | 8 | + | − | − | +4 days | − | 6 |
| − | 8 | + | − | − | +8 days | − | 6 |
| − | 8 | + | − | − | − | +4 days | 6 |
| − | 8 | + | − | − | − | +8 days | 6 |
| Healthy | 12 | − | − | − | − | − | 6 |
| Disease | 12 | + | − | − | − | − | 6 |
| − | 12 | + | +4 days | − | − | − | 6 |
| − | 12 | + | +12 days | − | − | − | 6 |
| − | 12 | + | − | +4 days | − | − | 6 |
| − | 12 | + | − | +12 days | − | − | 6 |
| − | 12 | + | − | − | +4 days | − | 6 |
| − | 12 | + | − | − | +12 days | − | 6 |
| − | 12 | + | − | − | − | +4 days | 6 |
| − | 12 | + | − | − | − | +12 days | 6 |

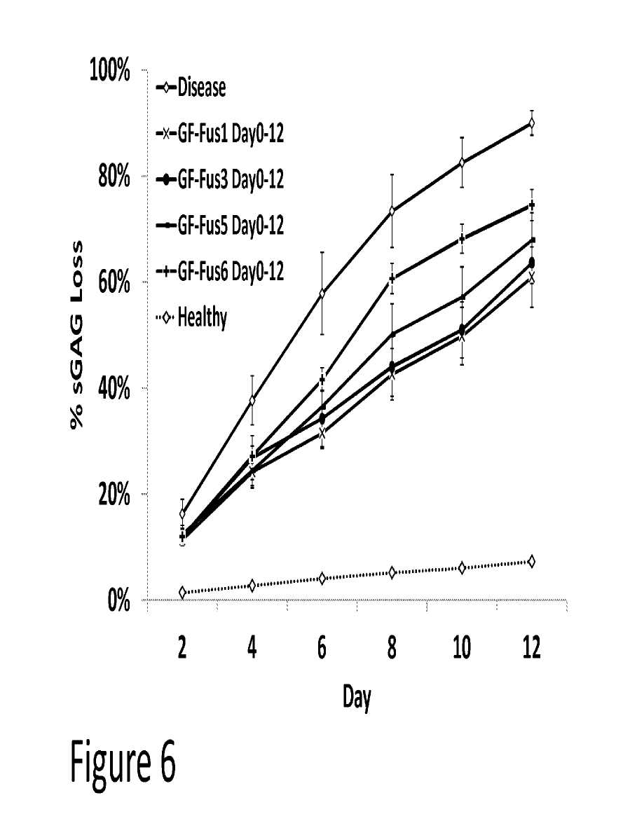

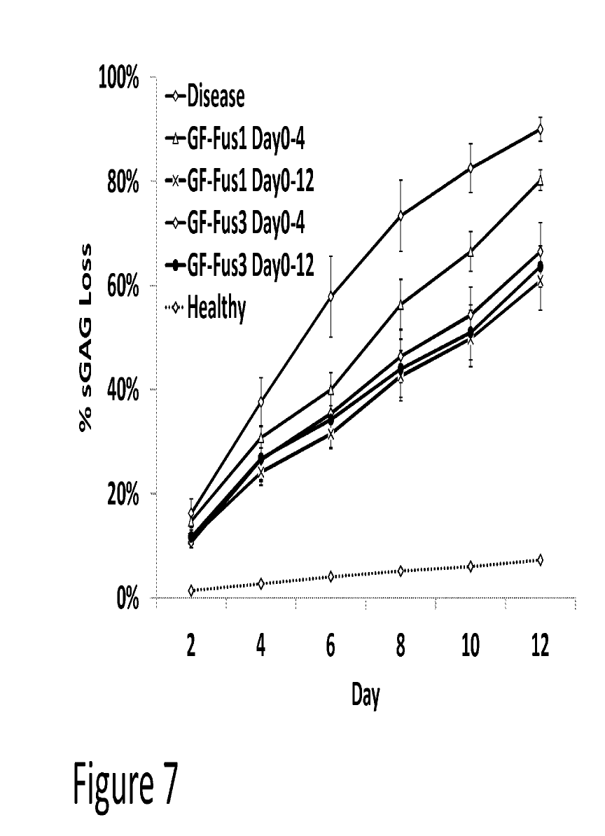

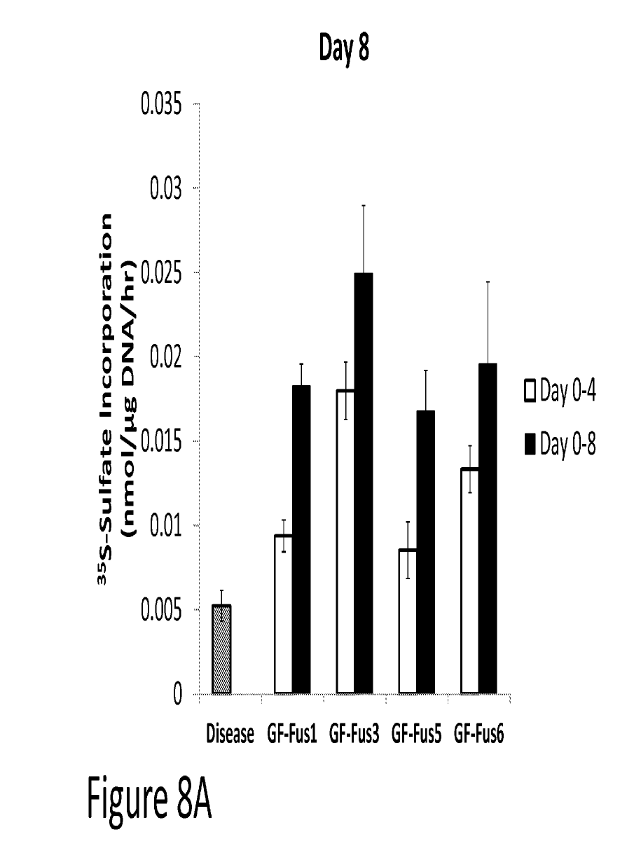

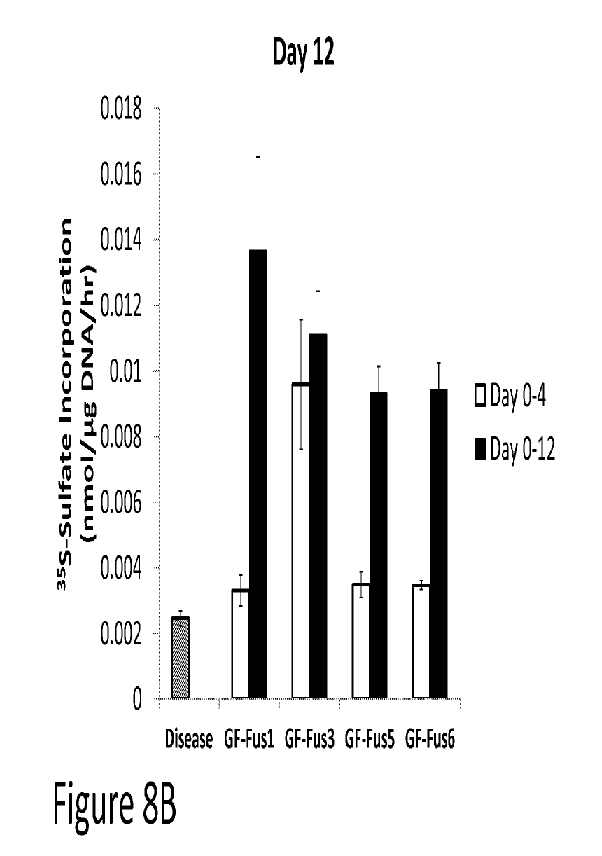

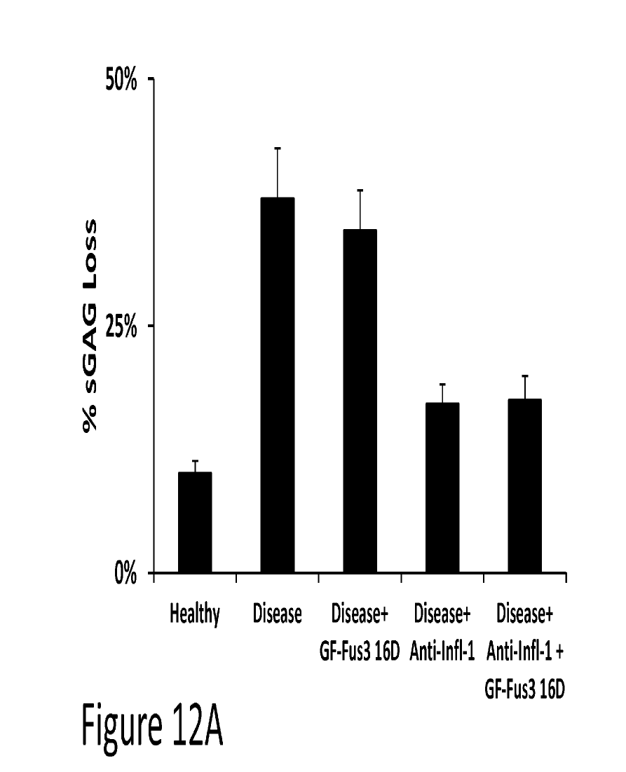

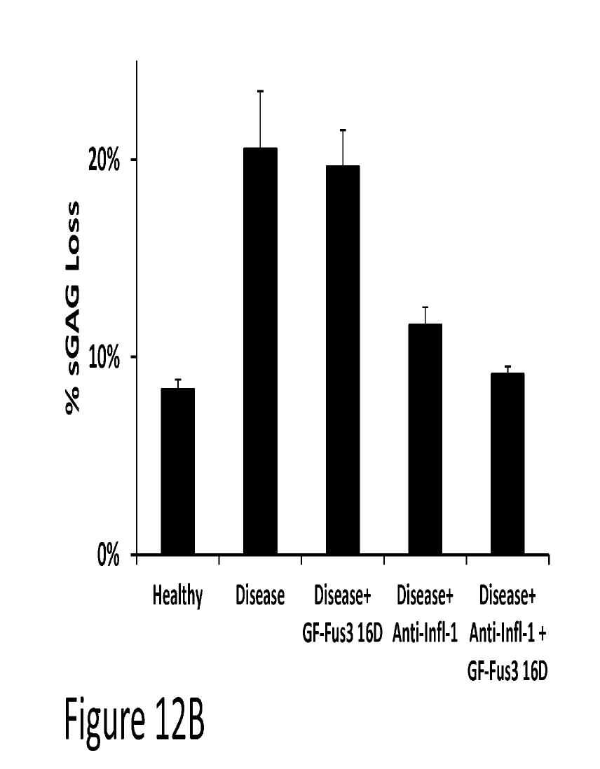

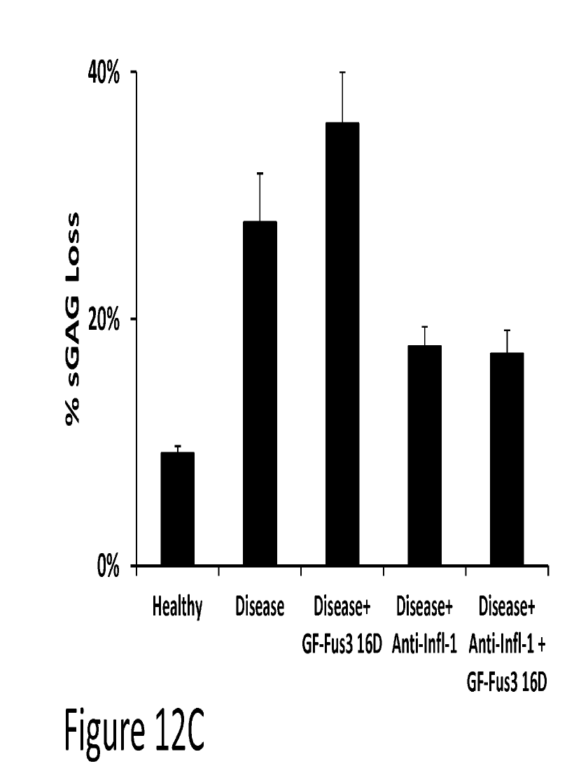

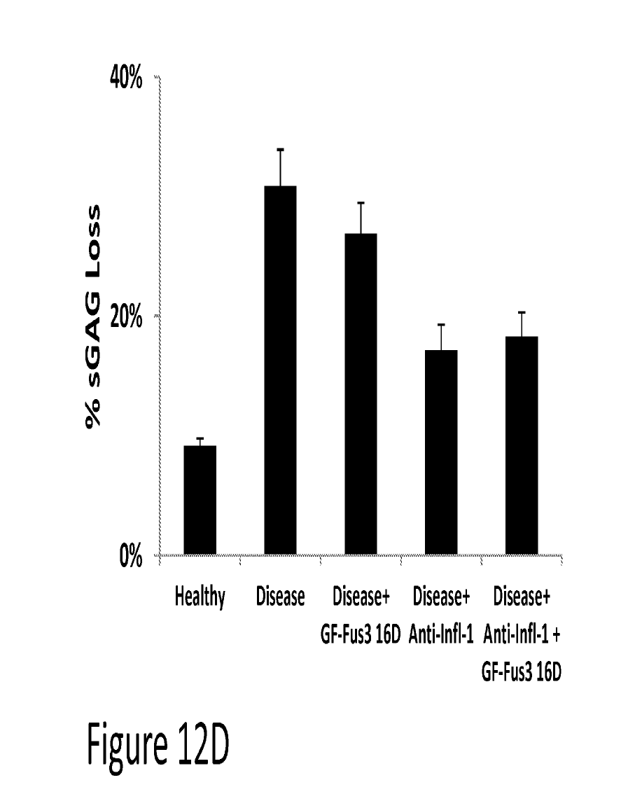

FIG. 6 shows cartilage matrix loss (% sGAG loss) against time (days) where cartilage matrix loss from bovine explants was reduced by each of the IGF fusion proteins tested (GF-Fus 1, 3, 5, and 6) relative to the Disease control. FIG. 7 shows sGAG loss against time (days) where cartilage matrix loss from bovine explants was reduced by 12 days of treatment with each of the IGF fusion proteins tested, relative to the no treatment control. Furthermore, for GF-Fus3, 4 days and 12 days of treatment reduced sGAG loss by an equivalent amount. However, for GF-Fus1 (a fusion protein without the Prelp heparin binding domain) 4 days of treatment resulted in a higher sGAG loss than 12 days of treatment. FIGS. 8A and 8B show cartilage matrix synthesis (³⁵S-sulfate incorporation) at day 8 and day 12, respectively, in bovine cartilage explants. Cartilage matrix synthesis is increased by both 8 (FIG. 8A) and 12 (FIG. 8B) days of treatment (black bars) with each of the IGF fusion proteins tested, relative to the Disease control. For GF-Fus3, 4 days and 12 days of treatment increased proteoglycan biosynthesis by an equivalent amount demonstrating sustained stimulation of cartilage matrix synthesis for 8 days of culture in medium without GF-Fus3 (FIG. 8B).

### Example 7

### Activity of the Combination of GF-Fus 3 with Dexamethasone (Anti-Infl-1) in an In Vitro Joint Disease Model Using Explanted Bovine Explant

The combination of GF-Fus3 (6×-Histidine tagged) with dexamethasone was characterized using the methods as described in Examples 5. The experimental design is summarized in Table 8 herein. All conditions used explants harvested from a single animal.

TABLE 8
Experimental design

| | Time | | | | |
|---|---|---|---|---|---|
| Control | point | IL1a | Dexamethasone | GF-Fus3 | # explants (n) |
| Healthy | 8 | − | − | − | 6 |
| Disease | 8 | + | − | − | 6 |
| — | 8 | + | +4 days | − | 6 |
| — | 8 | + | +8 days | − | 6 |
| — | 8 | + | − | + | 6 |
| — | 8 | + | +8 days | + | 6 |
| Healthy | 12 | − | − | − | 6 |
| Disease | 12 | + | − | − | 6 |
| — | 12 | + | +4 days | − | 6 |
| — | 12 | + | +12 days | − | 6 |
| — | 12 | + | − | + | 6 |
| — | 12 | + | +12 days | + | 6 |

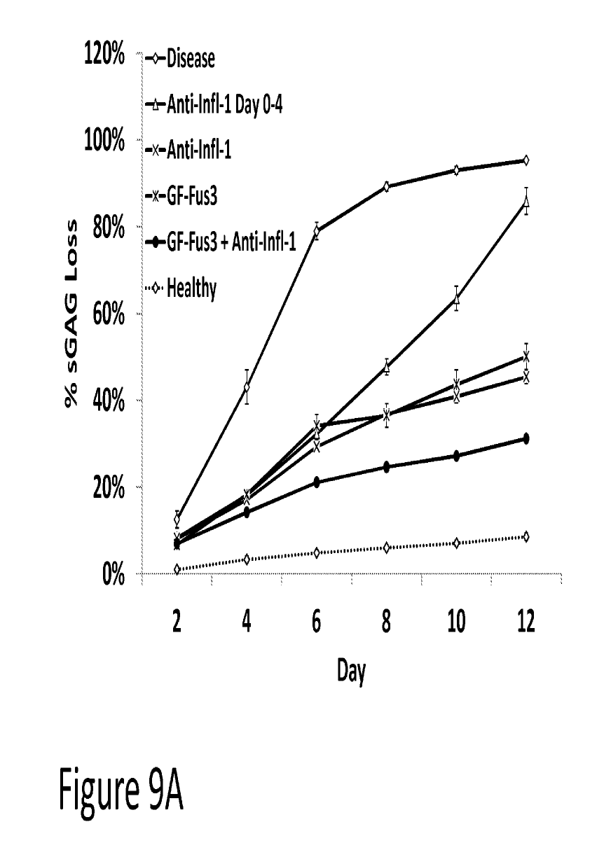

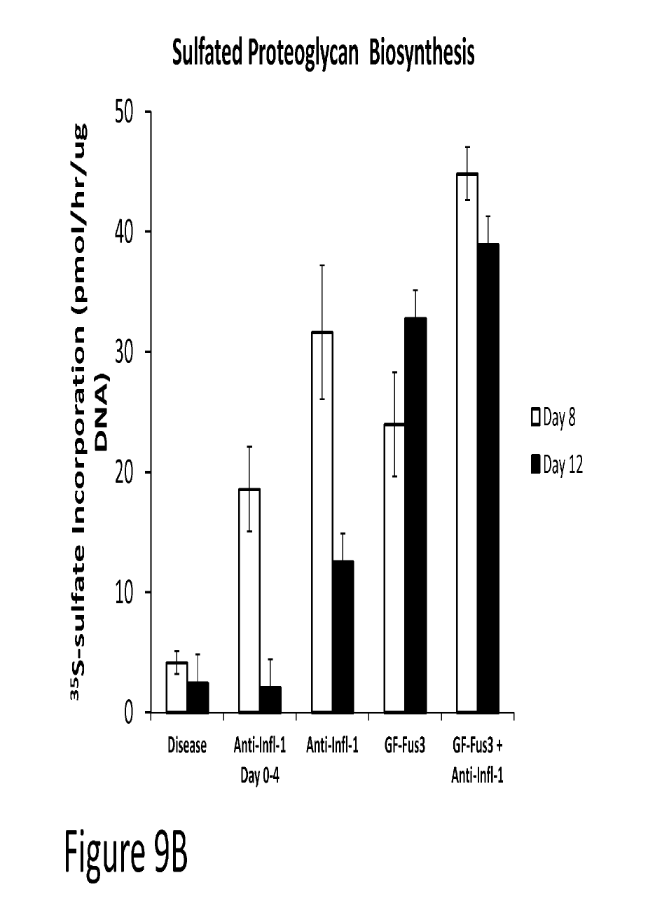

FIG. 9A shows cartilage matrix loss (% sGAG loss) against time (days). The combination of GF-Fus3 with dexamethasone was more effective at inhibiting IL-1 induced matrix loss in bovine explants than either GF-Fus3 or dexamethasone administered alone. FIG. 9B shows cartilage matrix synthesis (³⁵S-Sulfate incorporation) during the final 48 hours for cultures terminated at days 8 and 12. The combination of GF-Fus3 with dexamethasone was more effective at stimulating cartilage matrix synthesis in bovine explants than either GF-Fus3 or dexamethasone administered alone. The term Anti-Infl-X refers to different versions of dexamethasone where X=1 is dexamethasone, X=2 is dexamethasone-21-palmiate, and X=3 is dexamethasone phosphate.

### Example 8

### Sustained Release of GF-Fus2 from Methylcellulose Hydrogels with or without Hyaluronic Acid

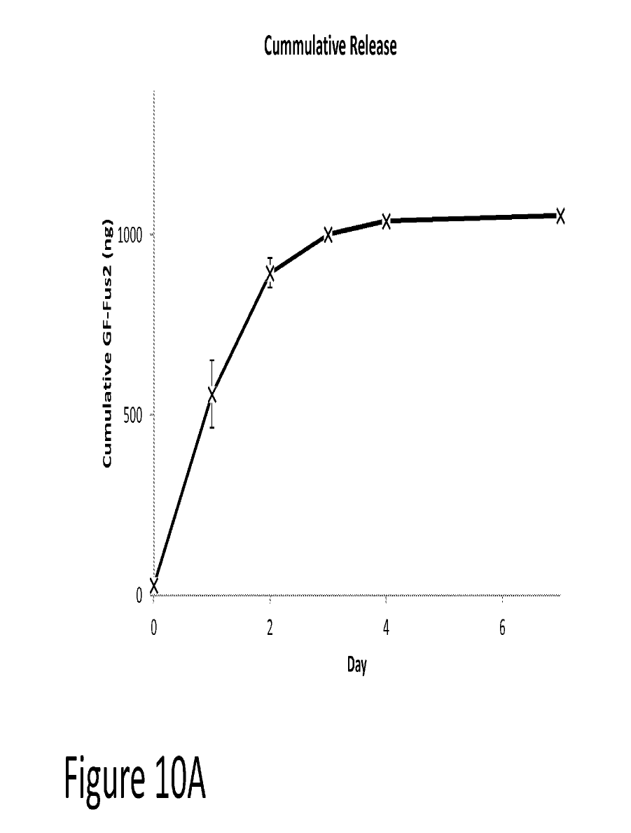

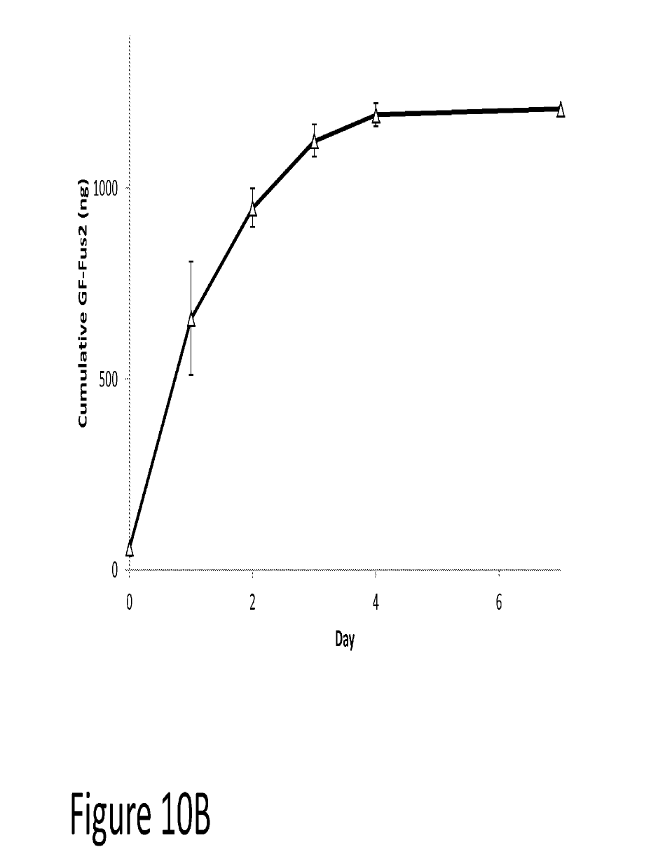

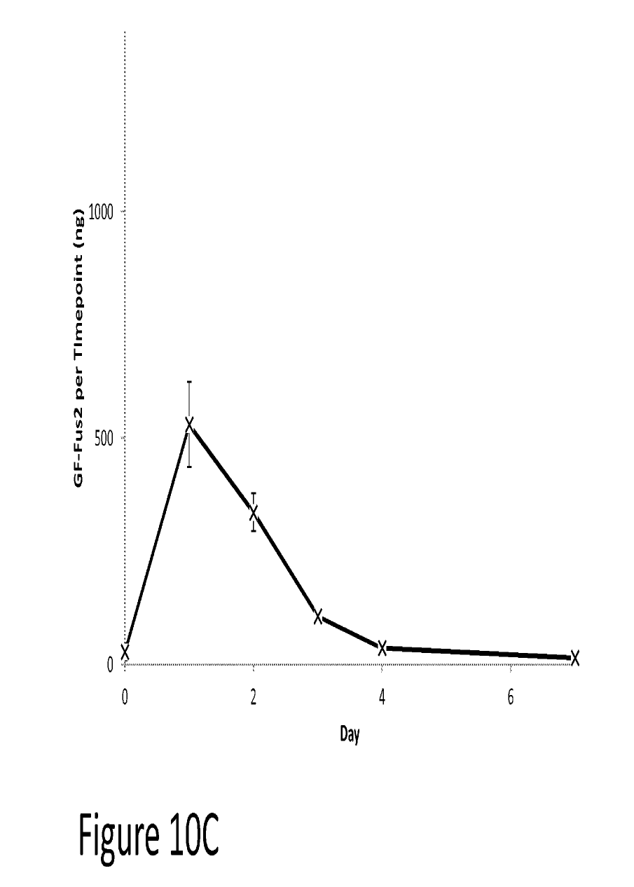

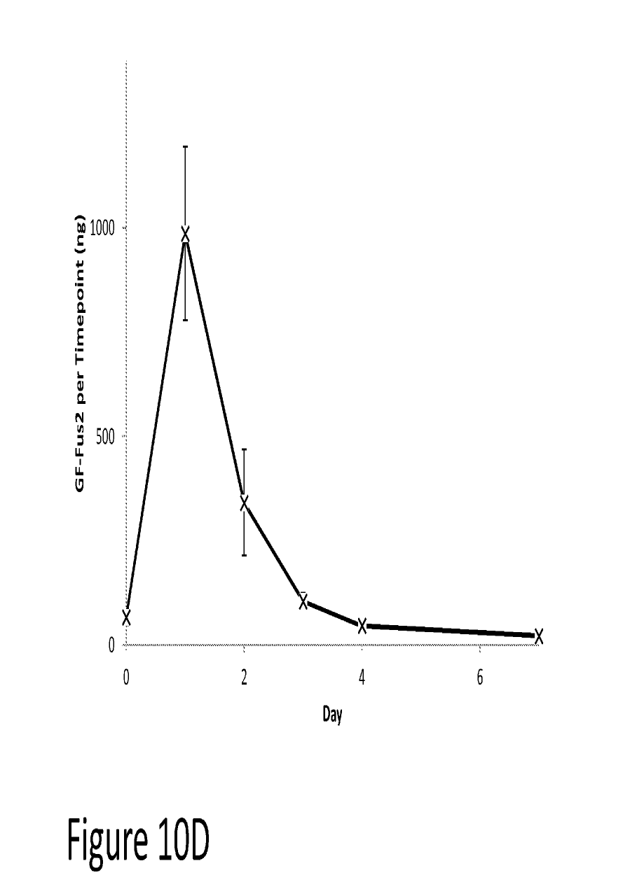

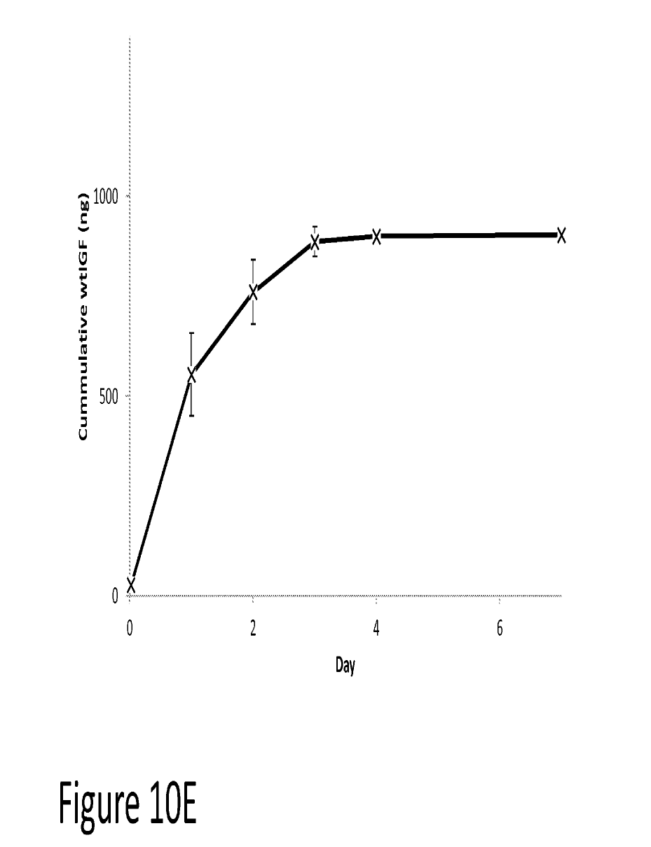

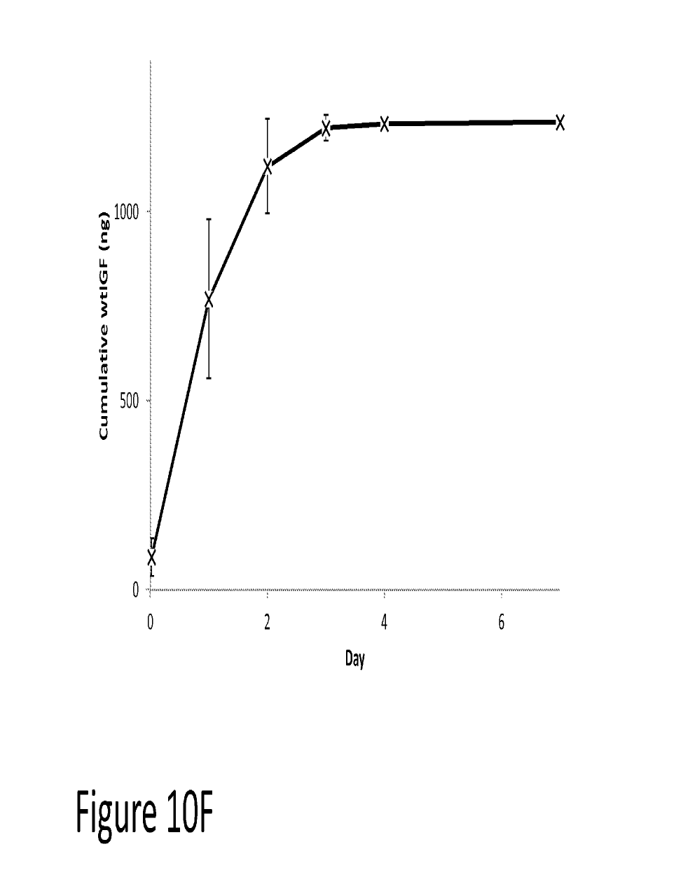

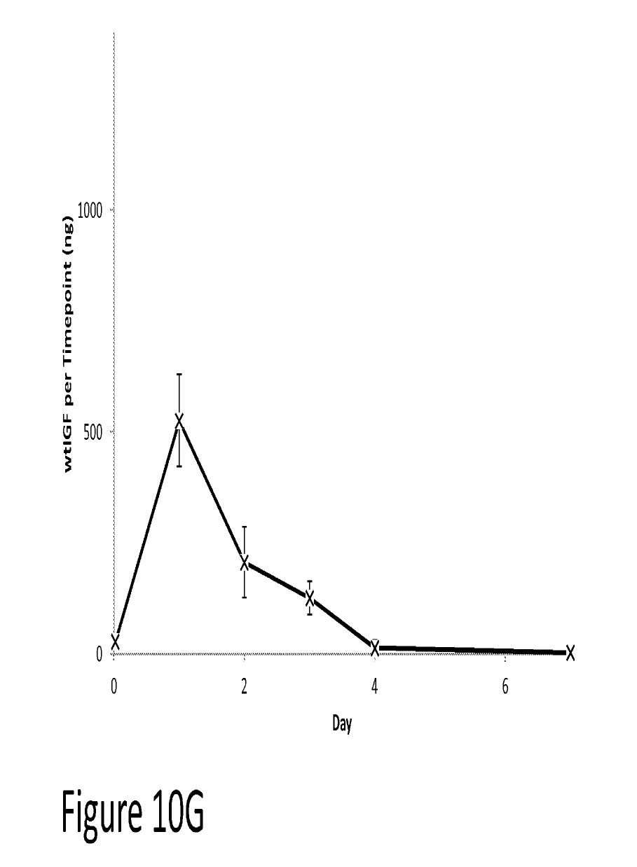

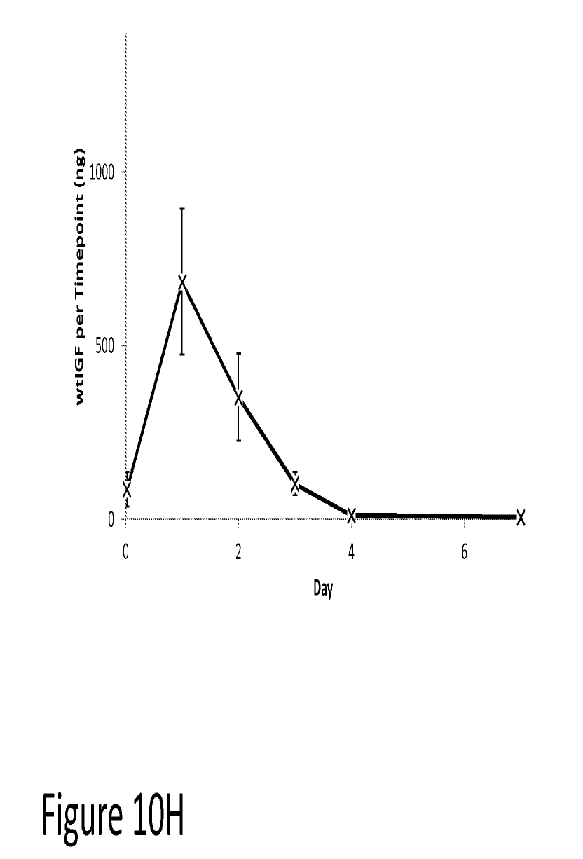

The in vitro sustained release of GF-Fus2 and wt-IGF from hydrogel formulations was assessed. Methylcellulose hydrogel, Gel 3 (6.1% (w/w) methylcellulose A15 (Sigma M7140) in HBS), and hyaluronan methylcellulose hydrogel, Gel 4 (1.8% (w/w) sodium hylauronate (Lifecore HA1M) and 6.1% methylcellulose in HBS buffer), were employed. Specifically, gels were cast in 50 μL total volume in flow cytometry tubes, with each gel containing 1 ug protein. Gels were incubated at 37° C. with agitation for 14 days in artificial synovial fluid (SF) (IgDMEM+Penicillin-Streptomycin+2.5% bovine serum albumin, ThermoSci Cat#37525). Artificial synovial fluid was assayed (6 repeats per condition) after 30 min, and thereafter on days: 1, 2, 3, 4, 7, 9, 11, 14. Protein release was determined using an anti-IGF ELISA (R&D Systems). The results are set forth in FIGS. 10A-H. GF-Fus2 and wild type IGF were released from both Gel 3 and Gel 4 at similar rates from day 0-3 with no further release after day 4 demonstrating sustained delivery of these proteins from the Gel 3 and Gel 4 over the first 3 days.

### Example 9

### Release of Dexamethasone-21-Palmitate (Anti-Infl-2) from Hydrogels with Embedded Lipid Nanoparticles

Hydrogels embedded with dexamethasone-21-palmitate containing lipid nanoparticles were produced and the release of dexamethasone-21-palmitate from these nanoparticles was measured.

Methylcellulose hydrogel, Gel 1 (9% (w/w) methylcellulose A15 (Sigma M7140) in HBS buffer (5 mM HEPES, 144 mM NaCl, pH 6.5)), and hyaluronan methylcellulose hydrogel, Gel 2 (2% (w/w) sodium hylauronate (Lifecore HA1M) and 7% methylcellulose in HBS buffer), were employed. Dexamethasone-21-palmitate lipid nanoparticles were produced with the lipid compositions set forth in Table 9.

TABLE 9
Dexamethasone-21-palmitate lipid nanoparticle composition (mg/mL particle
suspension in HBS)

| | | | Lipid Nanoparticle Type | | | | |
|---|---|---|---|---|---|---|---|
| Component | Source | Mol. wt | 1 | 2 | 3 | 4 | 5 |
| Dexamethasone- | TRC | 630.9 | 1 | 1 | 1 | 1 | 0 |
| 21-palmitate | D298830 | | | | | | |
| HSPC | Lipoid | 783.7 | 12.44 | 12.44 | 12.44 | 12.44 | 12.44 |
| Cholesterol | AlfaAesar* | 386.7 | 0 | 3.07 | 0 | 3.07 | 3.07 |
| PEG(2000)-DSPE | Lipoid | 2788 | 0 | 4.43 | 4.43 | 0 | 4.43 |

| | | | | | |
|---|---|---|---|---|---|
| Actual Dexamethasone-21-palmitate | 1.028 | 1.035 | 1.013 | 0.972 | 0 |
| concentration by HPLC: | | | | | |

*Recrystallized from ethanol

Specifically, a lipid film was formed by rotoevaporation from chloroform solution at 65° C. with overnight drying at 120 mm Hg. The film was hydrated in sterile HBS buffer (5 mM HEPES, 144 mM NaCl, pH 6.5) with hand swirling at 65° C. and vortexing at max speed for 30 sec. Dexamethasone-21-palmitate gels were formed by mixing of the resulting multilamellar vesicles (MLVs) with ice-chilled 1.1× gel stock to achieve a dexamethasone-21-palmitate concentration of 100 μg/g of the gel, except MC gel with 10512-4 which contains 97.2 μg/g gel.

Gels were dispensed into pre-weighed autoclaved 2-ml polypropylene cylindrical shell vials (National Scientific C4011-77P) with ethanol-treated polyethylene lids and allowed to form a “knob” on the bottom, then hardened at 37° C. for more than 24 hours. 5×300 mg gels were dispensed per lipid nanoparticle type per gel for a total of 50 gels. 700 μL of artificial synovial fluid consisting of the following components was added to each of the 50 vials: 1× Penicillin-Streptomycin, Gibco, Cat#15140-122; 2.5% BSA (Thermo Scientific, Cat#37525); and 1 g-DMEM, (Life Technologies, Cat#11885) and gels were incubated at 37° C. with gentle agitation. Artificial synovial fluid supernatant was removed and stored frozen at −80° C. and 700 μl of fresh artificial synovial fluid was added on days 1, 2, 3, 4, 7, 9.

Supernatants were analyzed by thawing, sonication and digestion with Lipase. Briefly, dexamethasone and dexamethasone-21-palmitate standard curves ranging from 320 nM to 5 nM, and including a zero point, were created in artificial synovial fluid to be treated in parallel with supernatants. 100 μL of these standards and supernatant from each gel were treated with 0.5% Triton-X-100. Treated samples and standards were placed in a 50° C. oven for 30 mins and then placed in a sonicator at room temperature for 5 minutes. Samples and standards were treated with Lipase, Chromobacterium viscosum (EMD Chemical catalog #437707) at 4 μg/mL. Plates were sealed and incubated at 37° C. overnight. Digests were analyzed using a dexamethasone ELISA kit from Neogen (catalog #101519) as follows: enzyme conjugate, wash buffer, and K-Blue substrate were used according to the instructions. A fresh standard curve of dexamethasone was created in artificial synovial fluid in the same range as above and left untreated as a control. Extra artificial synovial fluid was added to the standard such that the total volumes of the treated and untreated standards were identical. Samples and standards were either diluted 1:20 in EIA buffer (Neogen catalog #301277), or first samples were diluted 1:100 in artificial synovial fluid and then samples and standards were diluted 1:20 in EIA buffer. Samples and standards were placed into ELISA plates with enzyme conjugate for 45 mins at room temperature. Plates were washed 3 times with 300 μL wash buffer per well and inverted and tapped dry after each wash. 100 μL K-blue substrate was added and plates were incubated at room temperature for 30 mins 100 μL TMB stop solution (Kirkegaard & Perry Laboratories, Inc, Cat#50-85-06) was added to each well and plates were read at 450 nm on a Perkin Elmer Envision plate reader. Data from both samples diluted 1:100 and 1:20, and for 1:20 alone were regressed to the standard curve. Data from the 1:100 dilution was used unless the reading was below 1 ng/mL.

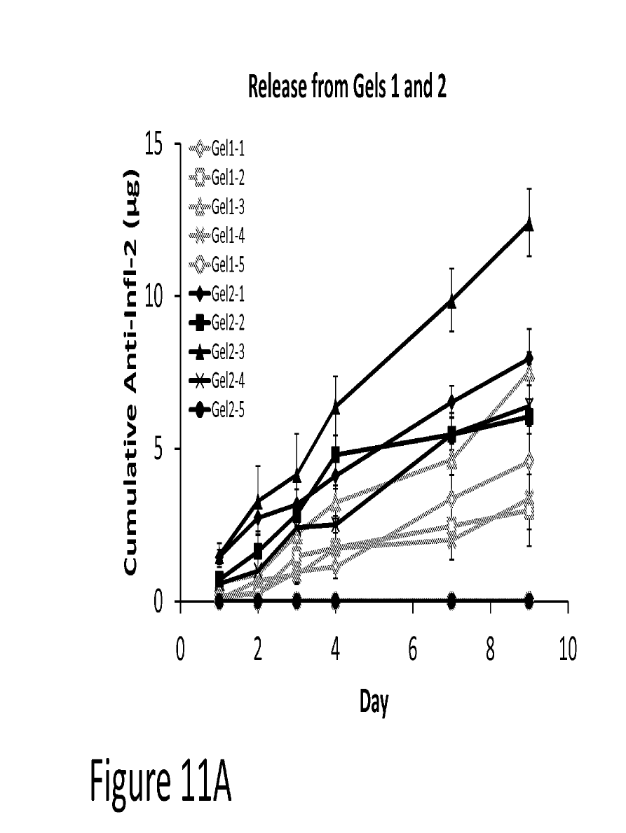

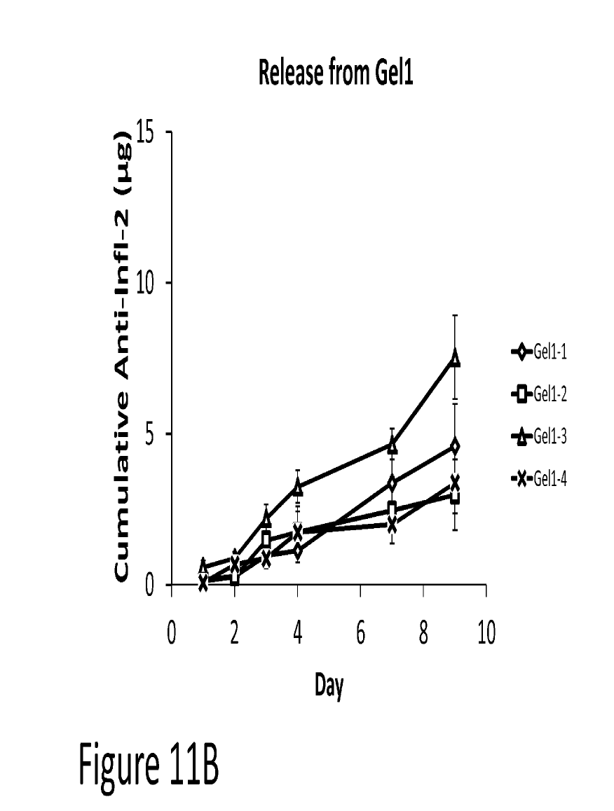

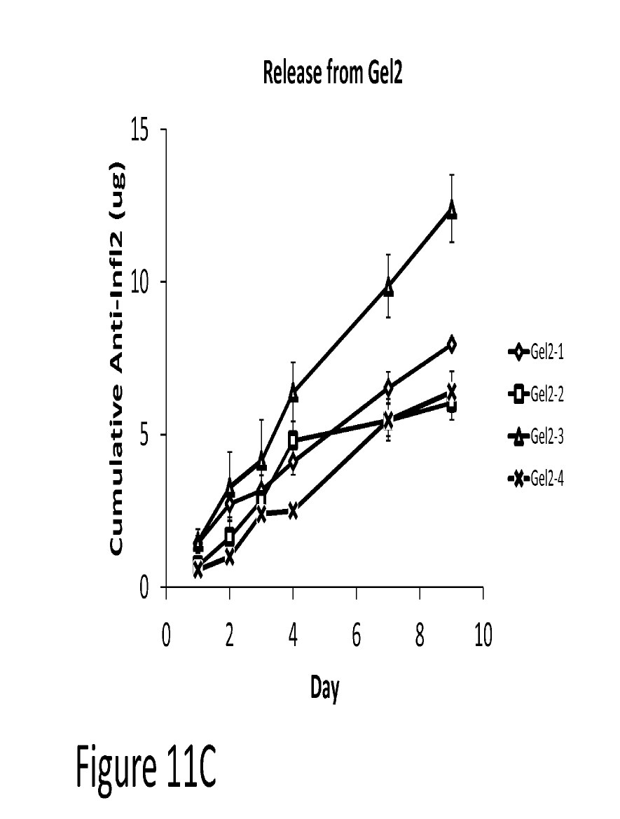

FIGS. 11 A, B, and C depict graphs of cumulative release of Anti-Infl-2 (dexamethasone-21-palmitate) against time (days) from hydrogel formulations Gel 1 or Gel 2 comprising lipid nanoparticle type 1, 2, 3, 4, or 5 as disclosed herein using the naming convention GelX-Y, where X is 1 or 2 for Gel 1 and 2, respectively, and Y is 1-5 to indicate nanoparticle type. The release rate of Anti-Infl-2 from these hydrogels is set forth in Table 10 herein.

The cumulative release at day 9 of Anti-Infl-2 for the slowest releasing formulations (Gel1-1 and Gel1-3) was approximately 4-fold lower than for the fastest releasing formulation (Gel2-3). Thus, by changing the composition of the gel and nanoparticle type the release rate of Anti-Infl-2 can be modulated.

TABLE 10
Release rate of Dexamethasone-21-palmitate from hydrogel

| | |
|---|---|
| | Release Rate |
| Nanoparticle | (ng/day) |

| Type | Gel 1 | Gel 2 |
|---|---|---|
| 1 | 582 | 797 |
| 2 | 356 | 659 |
| 3 | 834 | 1349 |
| 4 | 370 | 763 |

### Example 10

### How Uptake into Bovine Articular Cartilage of ¹²⁵I-Labeled GF-Fus3, GF-Fus1 and Wild-Type IGF Will be Determined

The partition coefficient, binding affinity and number of binding sites for GF-Fus3, GF-Fus1 and wild-type IGF in bovine and human articular cartilage is evaluated using methods previously described in: Garcia et al., Arch Biochem and Biophys 415 (2003) 69-79; Bhakta et al., J Biol Chem 275:8 (2000) 5860-5866; Byun et al., Arch of Biochem and Biophys 499 (2010) 32-39.

Briefly, bovine or human cartilage disks, 3 mm diameter by 400-2000 mm thick, are cored from cartilage slices using a dermal punch. These disks are subsequently distributed evenly into groups from among the different harvest sites on the joint and placed in fresh PBS (pH=7.4, containing protease inhibitors, Roche Cat#04 693 124 001) Immediately before use, ¹²⁵I-IGF-I (or ¹²⁵I-labeled GF-Fus3 or GF-Fus1) is purified to remove degraded fragments or free radioactivity as follows. The ¹²⁵I-protein is loaded onto a 0.6×30-cm Sephadex G50 column and eluted with a buffer consisting of PBS (pH=7.4) with 0.01 M acetic acid 1 0.1% BSA to ensure removal of any small molecular weight radiolabel. The void volume fractions corresponding to authentic labeled IGF-I or fusion protein are pooled.

A constant amount of ¹²⁵I-labeled protein (an average of 33 pM, specific activity 2000 Ci/mmol) and graded amounts of the corresponding unlabeled protein (0-200 nM) are then added to each group of disks. Following a 48-h incubation period at 37° C., the samples are briefly rinsed and then counted individually in a gamma counter along with the remaining buffers. The wet and dry weights of each disk are measured to determine water content. Dried samples are proteinase-K digested to assess glycosaminoglycan content as described in Example 5.

### Example 11

### Equine Osteoarthritis Model

The disease modifying activity of the fusion proteins disclosed herein will be characterized in an equine model of osteoarthritis. Suitable model systems include, without limitation, the model set forth in Mcllwraith et al. Bone Joint Res 2012; 1:297-309, which is incorporated herein by reference in its entirety. The subjects will be treated by intra-articular injection of IGF-fusion proteins with or without a glucocorticoid injection formulated for sustained retention or immediate release in the joint. Injection volume will be between 0.1-15 mL, with a concentration of dexamethasone palmitate in the injection volume between about 10 nM-10 mM. The concentration of IGF-fusion protein will be between 1 nM-1 mM. Between 1-10 injections will be given. If multiple injections are given, the time between injections will be between 3 days to 6 months.

To determine clinical outcomes, clinical examination of both forelimbs will be performed bi-weekly, including: lameness graded on a scale of 0 to 5 (0 being no lameness and 5 being non-weight-bearing lameness); and joint effusion graded on a scale of 0 to 4 (0 being no effusion and 4 being the most severe level).

Imaging can comprise: radiographs to observe features that may include radiological lysis, bony proliferation in the joint, and osteophytosis; CT imaging to observe changes that may include the volume of sclerotic bone in the trabecular area of the radial carpal bone; and MR imaging to observe changes that may include synovial fluid volume, synovial membrane proliferation, higher joint capsule thickening, joint capsule oedema, radial carpal bone oedema and radial carpal sclerosis.

Syno vial fluid will be collected at a frequency ranging from every 3 days to every month to assess the following outcomes: levels of synovial fluid protein, PGE2, CS846, CPII, sGAG, ColCEQ, C1,2C, osteocalcin, and Col-1. Serum levels of CS846, CPII, sGAG, osteocalcin, C1,2C and Col-1 will also be assessed.

### Example 12

### Sustained Activity of GF-Fus3 and Anti-Catabolic Activity of Dexamethasone (Anti-Infl-1) in an In Vitro Joint Disease Model Washout Experiment Using Explanted Human Cartilage

The activity of both the cartilage binding arm and the growth factor arm of GF-Fus3 (6×-Histidine tagged) was assessed in an in vitro joint disease model washout experiment using explanted human cartilage as described below. In addition, the anti-catabolic activity of Dexamethasone was determined both alone and in combination with both GF-Fus3 and GF-Fus1 (6×-Histidine tagged), which does not have a cartilage binding arm. This human cartilage joint disease model mimics the catabolic phase of joint damage driven by inflammatory cytokines that occurs after injury or in chronic disease. Treatments were also tested in the absence of inflammatory cytokines to assess their effect on cartilage tissue when inflammation is reduced or eliminated.

### Methods

Human cartilage explants are harvested from the knee and ankle of human cadaver donors within 24 hours postmortem. Joints are aseptically dissected by a pathologist to assess gross cartilage morphology by the modified Collins scale (Kuettner, et al., Cartilage degeneration in different human joints. Osteoarthritis Cartilage. 2005; 13(2):93-103) and only grade 0 or 1 joints are used. Full-thickness knee cartilage surfaces are harvested from the femoral-patellar groove and chondyles using a scalpel. Full thickness ankle cartilage is harvested from the dome of talus, proximal area under dome of talus, head of the talus, tibial malleolus and fibular malleolus using a scalpel. Using a 4 mm diameter disposable biopsy punch, full thickness cartilage cores are punched from the cartilage surfaces keeping the superficial zone intact and used as explants in culture.

Explants are cultured in 96-well plates in 300 μL of low glucose DMEM (1× Gibco 11885-084) with added Penicillin-Streptomycin (1×, Gibco 15140-122), ascorbic acid (20 μg/mL, Sigma A4403), proline (400 μM, Sigma P5607-256), non-essential amino acids (1×, Sigma M7145), and HEPES (100 mM, Gibco 15630-080). Explants from 2-3 donors (4-6 explants per donor) are used for each treatment condition for a total explant number of 12-18 per condition. Donor ages were: 67 year male ankle, 71 year male ankle, 76 year female ankle, 59 year male knee, 34 year male knee, and 63 year female knee. Medium is changed every 2 days. On day 14, the medium is supplemented with 5 μCi/mL of ³⁵S-sodium sulfate (Perkin Elmer NEX041H005MC, 5 mCi).

Time points are taken at day 16 as follows. Explant tissue is washed four times for 30 min each (2 hr total) in 1 mM unlabeled sodium sulfate in PBS. Each explant is weighed wet and frozen at −20° C. until digestion. Tissue digestion is performed with Proteinase K (Roche cat #3115879001) and each explant is digested in 1 mL 500 μg/mL Proteinase K in 50 mM Tris-HCL, 1 mM calcium chloride pH=8.0 buffer at 60° C. overnight. Measurement of sGAG content and DNA content is performed using standard assay methods such as those described in Hoemann, 2004, Methods in Molecular Medicine: Cartilage and Osteoarthritis. Totowa, N.J.: Humana Press Inc.; p. 127-52. ³⁵S-sulfate content of the digested cartilage explants is quantified by mixing 204, of digest with 250 μL of scintillation fluid (Perkin Elmer cat #1200-439) and counting with A WALLAC 1450 MICROBETA TRILUX scintillation counter.

### Results

Treatments were given at the following concentrations: GF-Fus1 13.3 nM=135 ng/mL; and GF-Fus3 13.3 nM=181 ng/mL; Dexamethasone 100 nM=39.2 ng/mL. Growth factor and steroid treatment conditions included the cytokines TNF-alpha (R&D Systems, Cat#210-TA, 25 ng/mL), IL-6 (R&D Systems, Cat#206-IL, 50 ng/mL), and IL-6R alpha (R&D Systems, Cat#227-SR, 250 ng/mL) which were supplied in each medium change. GF-Fus1 and GF-Fus3 were added to fresh medium for either the first 8 days (8 D) or the entire 16 day culture duration (16 D), whereas Dexamethasone was added for the entire 16 days in all cases. Controls were either no cytokines (Healthy) or with cytokines alone (Disease). The outcomes measured from the cartilage explants at day 16 were ³⁵S-sulfate incorporation, DNA, and sGAG content, and sGAG released to media for all media changes.

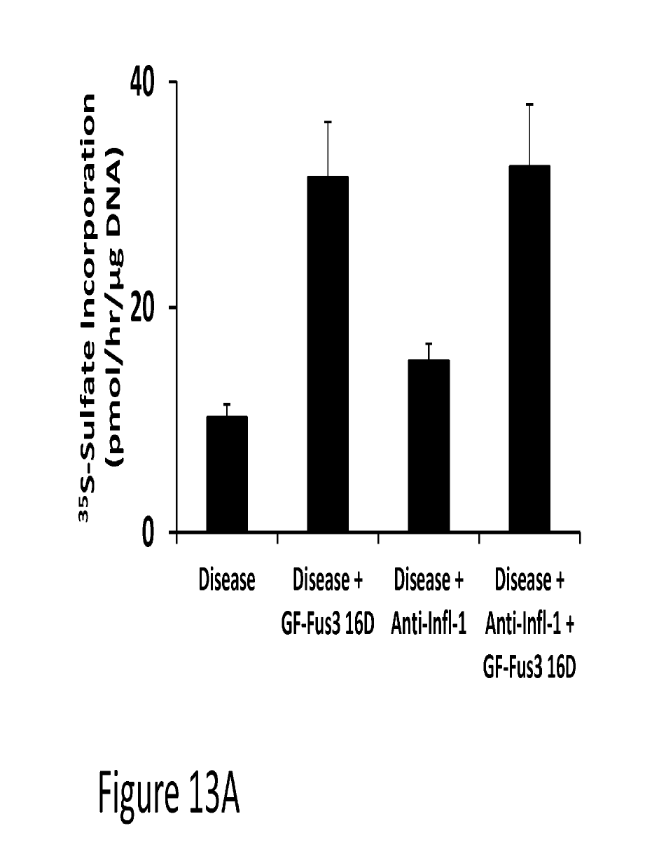

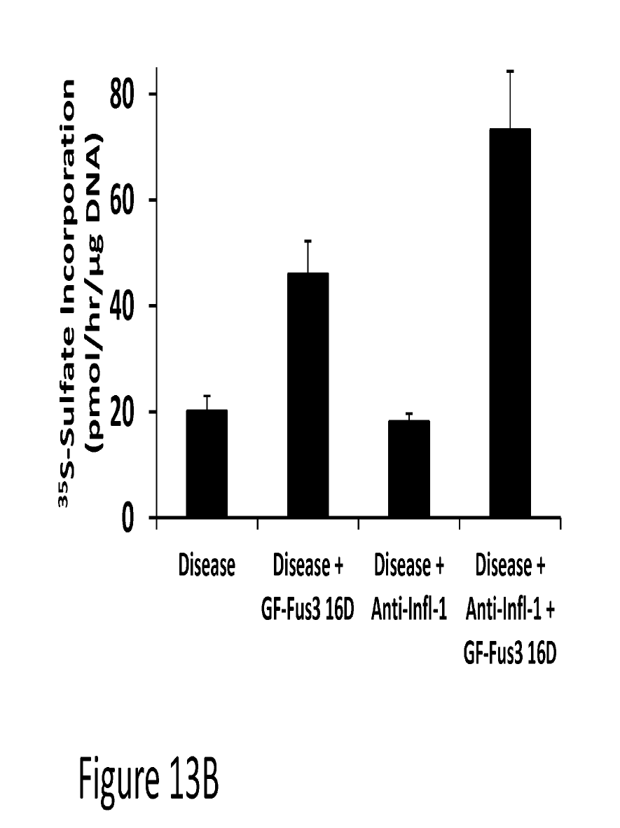

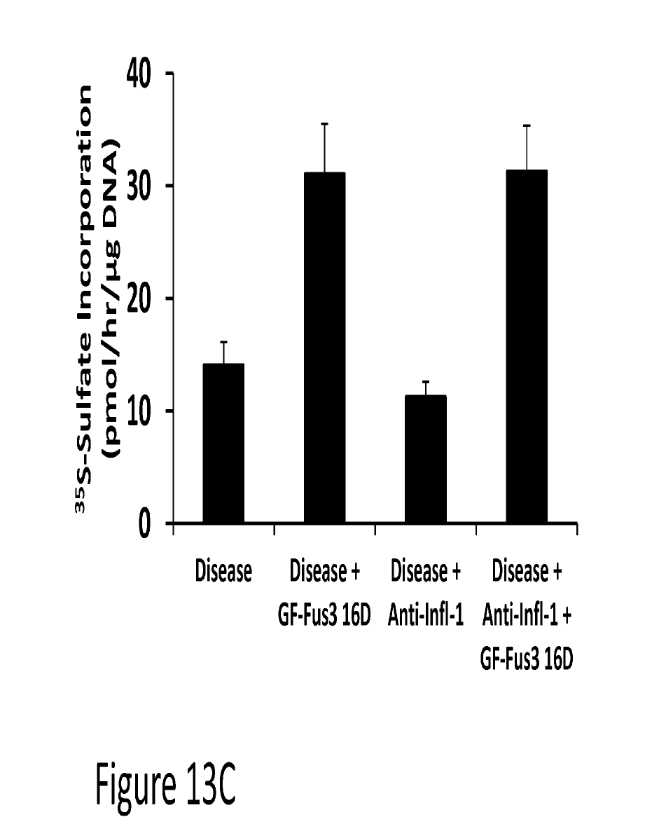

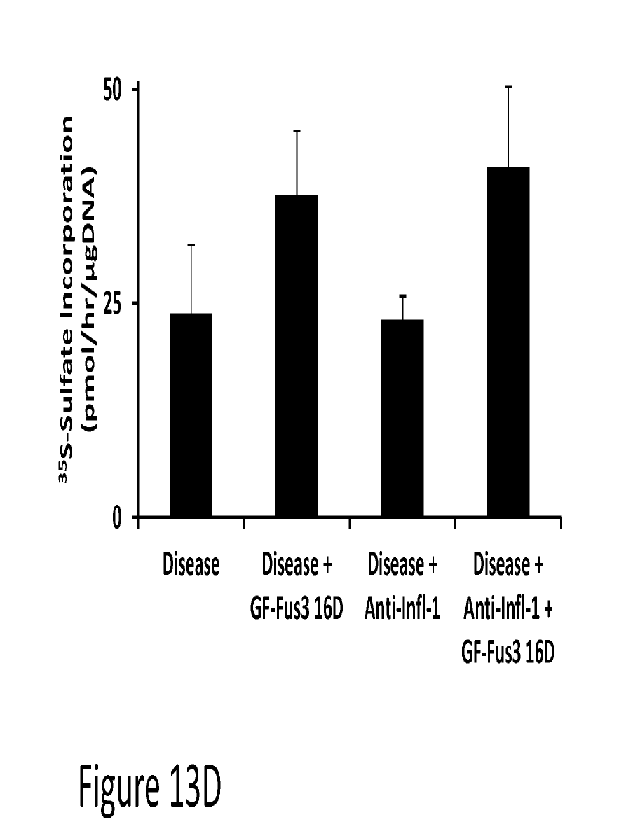

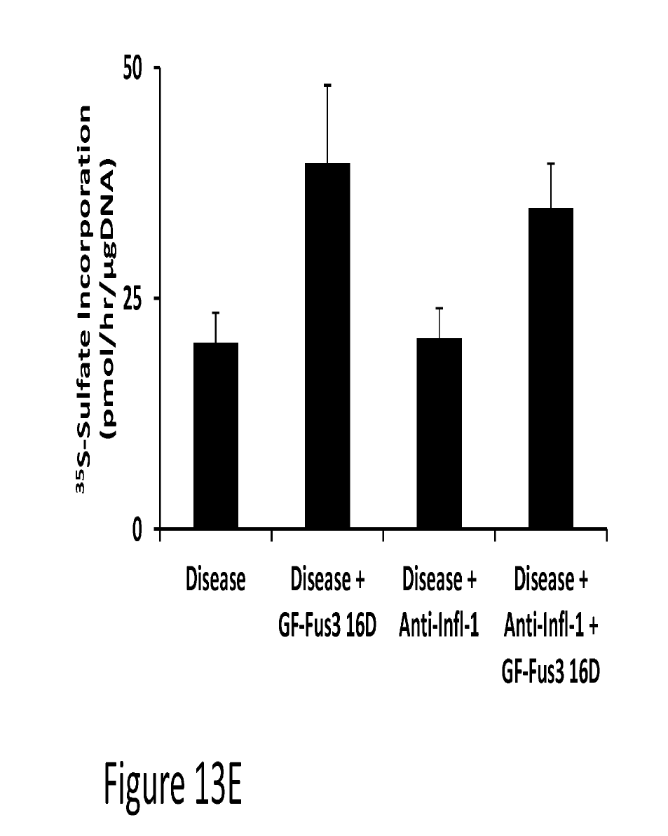

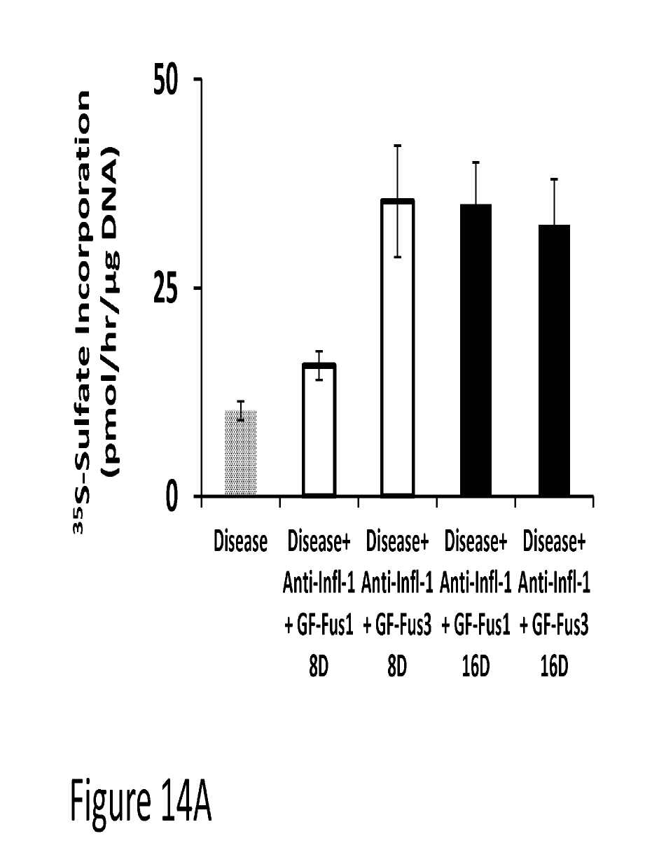

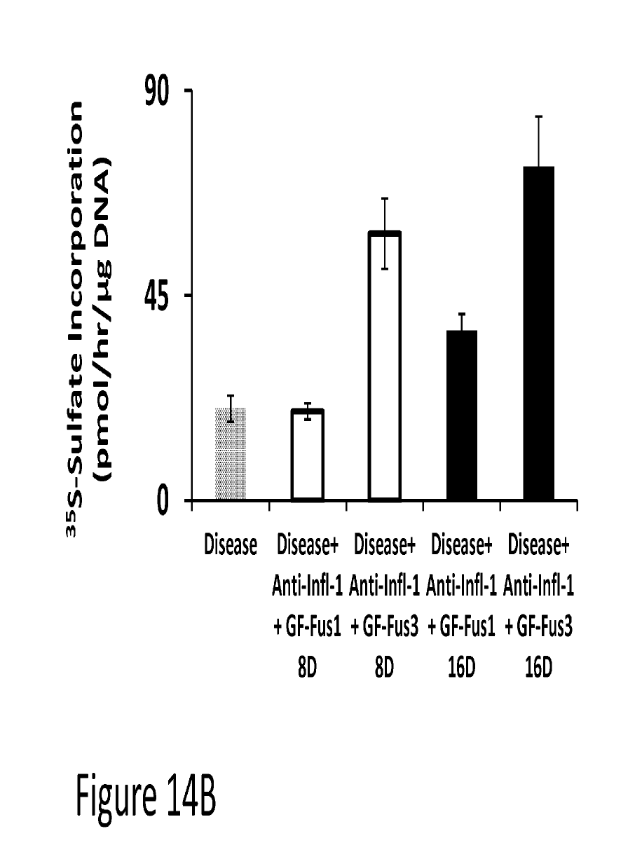

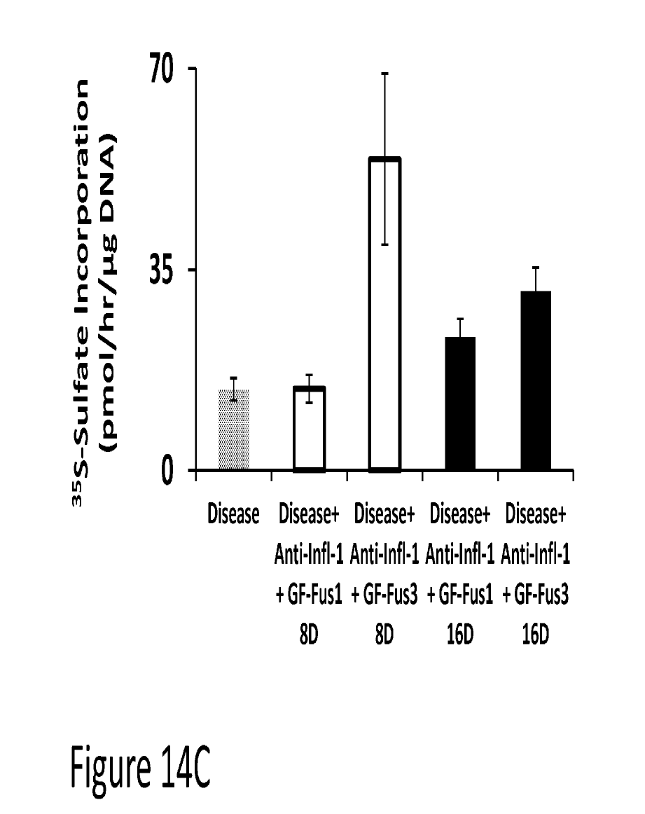

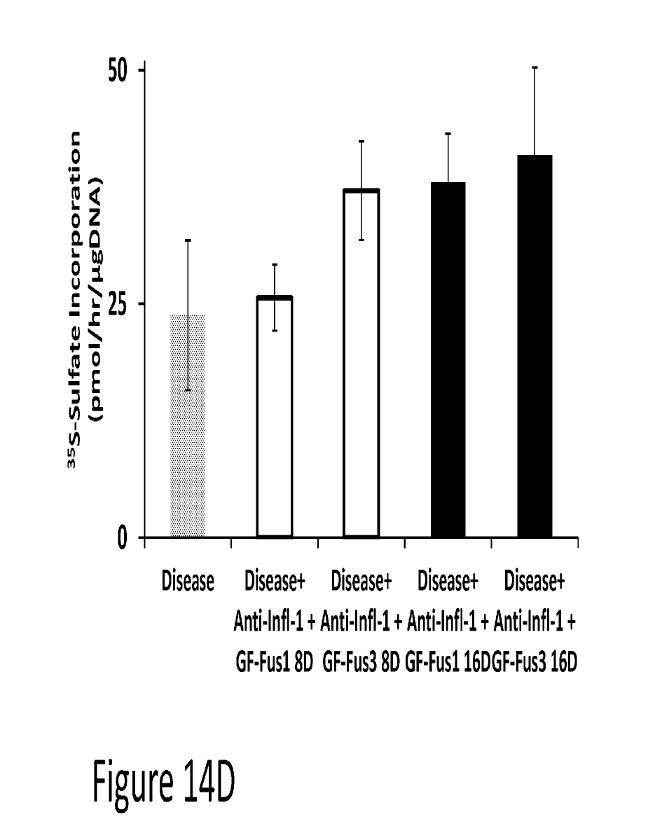

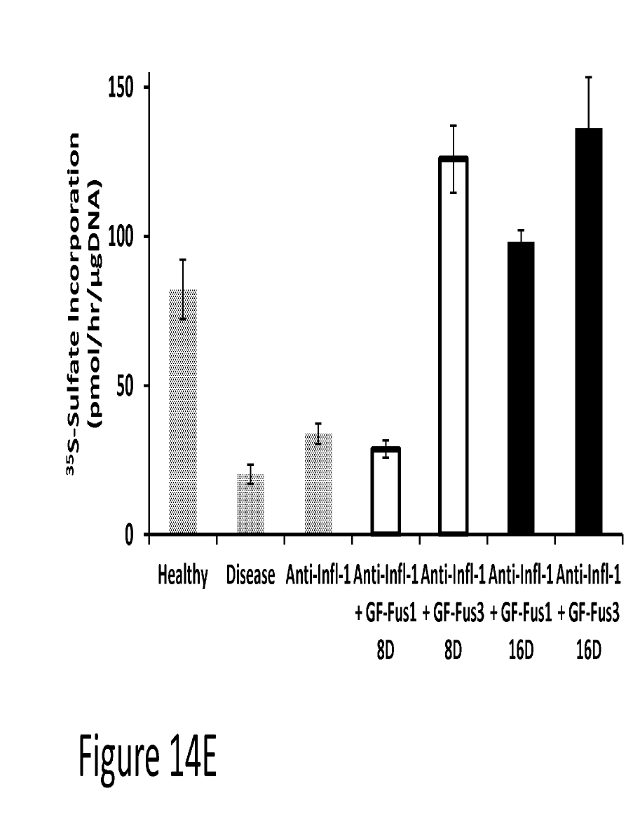

FIG. 12A-D shows that dexamethasone (Anti-Infl-1) reduces matrix catabolism of human ankle and knee cartilage compared to the Disease condition, both with and without the addition of GF-Fus3, suggesting the potential to protect cartilage from damage during cytokine driven disease in a highly translational model of human cartilage degradation. In addition, the robustness of this reduction in matrix loss is demonstrated by the consistent results for cartilage explants harvested from a range of anatomical sites in the ankle and knee (dome of talus, FIG. 12A, posterior talus, FIG. 12B, the head of the talus and the tibial and fibular malleolus, FIG. 12C, and femoral-patellar groove, FIG. 12D). FIG. 13A-E shows that GF-Fus3 upregulates the synthesis of new sulfated ankle and knee cartilage matrix compared to the Disease condition, both with and without the addition of Dexamethasone, suggesting the potential for cartilage repair with functional load-bearing matrix molecules. This effect is robust across cartilage harvested from a range of anatomical sites in the ankle and knee as well (dome of talus, FIG. 13A, posterior talus, FIG. 13B, the head of the talus and the tibial and fibular malleolus, FIG. 13C, femoral-patellar groove, FIG. 13D, and femoral chondyle, FIG. 13E). FIGS. 14A-D show that only GF-Fus3, but not GF-Fus1, sustains potential cartilage repair activity of human ankle and knee cartilage explants for 8 days in medium free of each respective protein (i.e. equivalence of white and black bars for GF-Fus 3 conditions, but not GF-Fus 1). This effect is robust across the dome of the talus (FIG. 14A), posterior talus (FIG. 14B), head of the talus and the tibial and fibular malleolus (FIG. 14C) and the femoral-patellar groove (FIG. 14D), where in all cases Disease+Anti-Infl1+GF-Fus1 8 D was similar to the Disease control while Disease+Anti-Infl1+GF-Fus3 8 D showed equivalent or superior potency to the continuously delivered proteins (i.e. Disease+Anti-Infl1+GF-Fus1 16 D and Disease+Anti-Infl1+GF-Fus3 16 D).

In the absence of active disease or recent acute injury, intra-articular cytokine levels may be reduced. Thus the activity of Dexamethasone alone and in combination with GF-Fus1 and GF-Fus3 was assessed in a cytokine free setting using human knee chondyle cartilage explants. FIG. 14E shows that in the absence of cytokines Dexamethasone reduces sulfated matrix biosynthesis, but the combination of GF-Fus1 or GF-Fus3 with Dexamethasone for the entire 16 day culture (black bars) restores sulfated matrix biosynthesis to a level at or above healthy controls. Furthermore, when GF-Fus1 (without a cartilage binding domain) and GF-Fus3 (with a cartilage binding domain fusion) are removed for the final 8 days of culture (white bars), only GF-Fus3 sustains sulfated matrix biosynthesis at the level equivalent to continuous 16 day treatment (unlike GF-Fus1) likely due to its increased binding, retention, and activity within human cartilage explants.

### Example 13

### Sustained Retention of Lipid Particle Encapsulated Dexamethasone, Lipid Particle Encapsulated Dexamethasone-21-Palmitate, and Immediate Release Dexamethasone Phosphate Mixed with Either GF-Fus3 or wtIGF after Intra-Articular Injection into Rat Knees

The retention after intra-articular injection into rat knees of 3 dexamethasone derivatives (see methods) was measured. Dexamethasone and dexamethasone-21-palmitate were encapsulated in lipid particles and compared to the non-encapsulated, soluble dexamethasone phosphate formulation, which is the currently marketed injectable molecular structure of dexamethasone. GF-Fus3, an engineered, cartilage-binding IGF fusion protein (without a His tag), was mixed with the dexamethasone-21-palmitate particle suspension and the dexamethasone phosphate solution to determine if the presence of lipid particles changed the retention of GF-Fus3 in the rat knee. Wild-type IGF was mixed with the dexamethasone particle suspension as a control to compare to the retention of GF-Fus3 (Table 12).

### Methods

The source of all materials is listed in Table 11. The formulations for Groups 1, 2, and 4 in Table 12 were prepared by rotoevaporation from chloroform solution at 60° C. with overnight drying at 110 μm Hg. The film was hydrated in sterile HBS-6.5 buffer (5 mM HEPES, 144 mM NaCl, pH 6.5) with hand swirling at 68° C. plus vortexing at max speed for 45-60 sec. The resulting lipid particle suspension was mixed 1:1 with the appropriate protein solution in 2×PBS at 2× the final concentration listed in Table 12. The formulation for Group 3 was prepared by making a sterile solution of dexamethasone phosphate in distilled water at 10× the concentration in Table 12. This was mixed 1:4 with HBS-6.5 and subsequently 1:1 with the appropriate 2× solution of protein in 2×PBS.

TABLE 11
Materials

| Material | Source | Cat # |
|---|---|---|
| Wild-type IGF-1 | R&D Systems | 291-G1 |
| LR3-IGF-PRELP (GF-Fus3) | Merrimack (in | |
| | house) | |
| HSPC | Lipoid | |
| Dexamethasone (Anti-Infl-1) | Sigma | D9184 |
| Dexamethasone 21-palmitate (Anti-Infl-2) | Toronto Research | D298830 |
| | Chemicals | |
| Dexamethasone phosphate (Anti-Infl-3) | Sigma | D1159 |
| PBS | Life Technologies | 10010-031 |
| Chloroform HPLC-grade alcohol- | EMD | CX1058-1 |
| stabilized | | |

TABLE 12
Injection Formulation

| Group | Drug/Pro-Drug | Protein | HSPC |
|---|---|---|---|
| Group 1 | Dexamethasone-21-palmitate | GF-Fus3 | 4.78 mg/mL |
| | (1.94 mM) | (63.9 μM) | |
| Group 2 | Dexamethasone (1.94 mM) | Wild-type IGF | 4.78 mg/mL |
| | | (77.1 μM) | |
| Group 3 | Dexamethasone phosphate | GF-Fus3 | None |
| | (1.94 mM) | (63.9 μM) | |
| Group 4 | none | None | 4.78 mg/mL |

Lewis rats (>275 grams) were administered 50 uL of the drug formulations in Table 12 by intra-articular injection into the right knee. Six rats were injected per condition per time point for a total of 96 rats. For Groups 1-3, animals were sacrificed immediately and at 1 hour, 4 hours, 24 hours, and 96 hours after injection. For Group 4, animals were sacrificed at 1 hr after injection only. Animals were anesthetized with isofluorane and bled through the descending aorta into a vacutainer to collect serum. Right knees were lavaged with 100 μL of saline. The cartilage, meniscus, cruciate ligament, and patella with surrounding synovium were collected, snap-frozen, and stored at −80° C. Cartilage, meniscus, ligament, and patella with surrounding synovium samples were pulverized in Covaris Tissue Tubes (Cat #520001) using a Covaris CryoPrep instrument after chilling in liquid nitrogen. Pulverized samples were suspended in Tissue Extraction Reagent (Life Technologies, Cat# FNN0071) (50 μL for cartilage, 100 μL for ligament, 200 μL for meniscus, and 400 μL for patella) and mixed on a rotary shaker at 4° C. for 12-18 hours. Lysates were centrifuged at 4000 g and clarified supernatants were removed.

Aliquots of clarified lysate for each tissue type, lavage, and serum samples were treated with Triton-X-100 and Lipase as described in Example 9 with alkaline phosphatase (Sigma-Aldrich cat# P0114) added at the same time as the lipase per the manufacturer's instructions. Treated lysates were diluted 1×, 10×, 100×, 1000×, and 10,000× in Tissue Extraction Reagent. Treated lavage and serum were diluted 1×, 10×, 100×, 1000×, and 10,000× in artificial synovial fluid. All dilutions were analyzed by dexamethasone ELISA (Neogen cat#101519) as described in Example 9.

Aliquots of clarified lysate samples were diluted 10×, 100×, 1000×, 10,000×, and 100,000× (lavage at immediate time point only) into PBS with Tween 20 (Sigma-Aldrich cat#274348, final concentration 5% v/v Tween 20, 10% Tissue Extraction Reagent in all dilutions). Lavage and serum samples aliquots were diluted 10×, 100×, 1000×, 10,000×, and 100,000× (lavage at immediate time point only) into PBS with Tween 20 (final concentration 5% v/v Tween 20, 10% Artificial Synovial Fluid in all dilutions). All dilutions were analyzed by human IGF-1 ELISA (R&D Systems Cat# DY291). The kit protocol was followed with the following exceptions: 384-well black microplates and SuperSignal ELISA Pico Chemiluminescent Substrate (Thermo Scientific Cat#37069) were used and the plates were read on a luminometer at 450 nm.

### Results

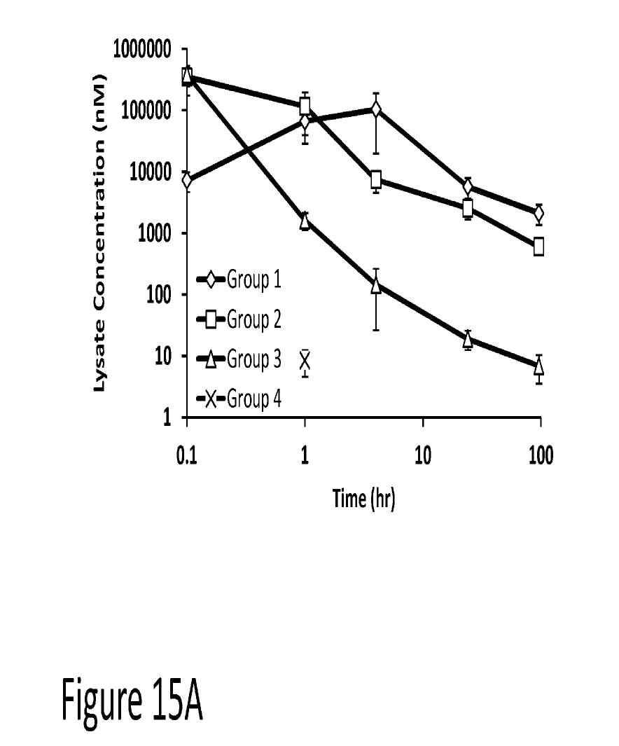

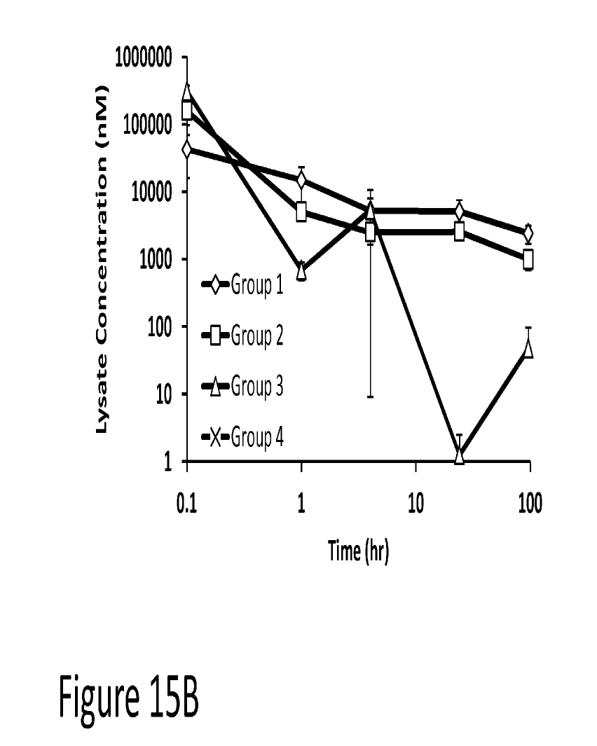

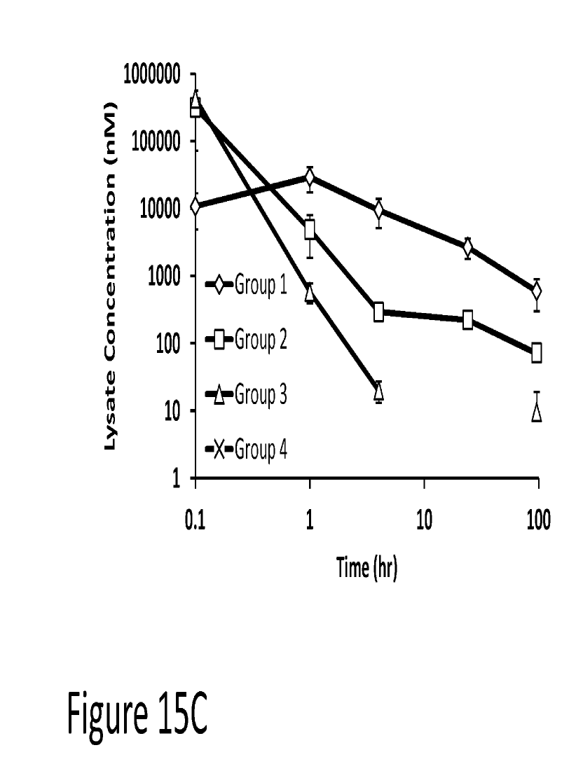

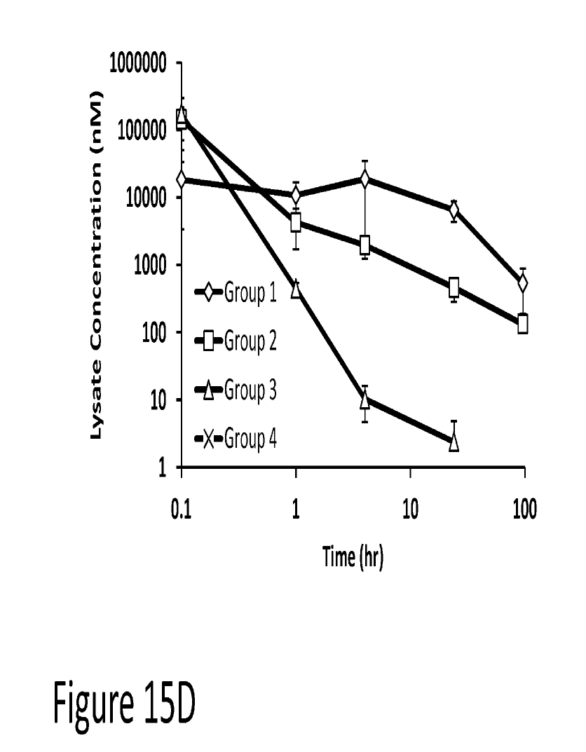

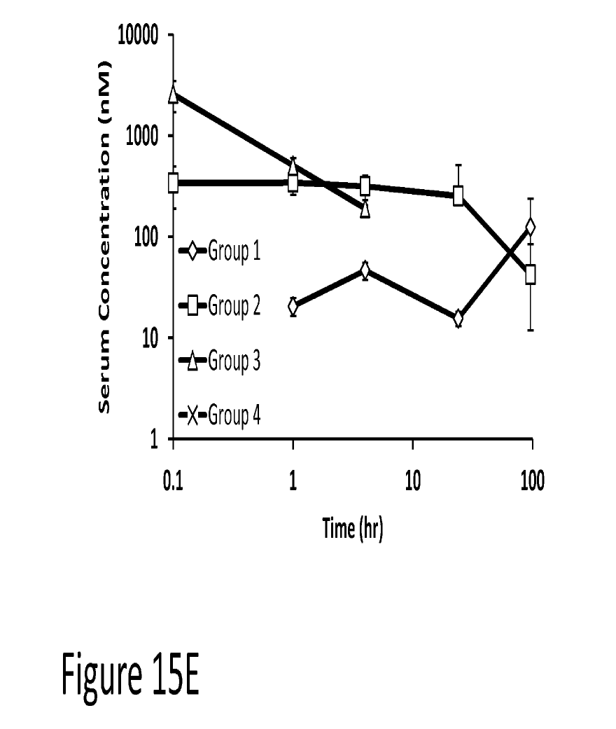

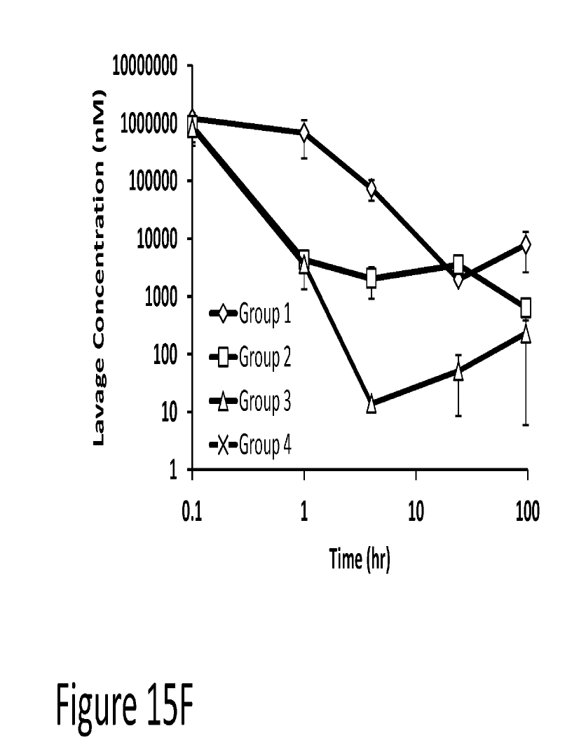

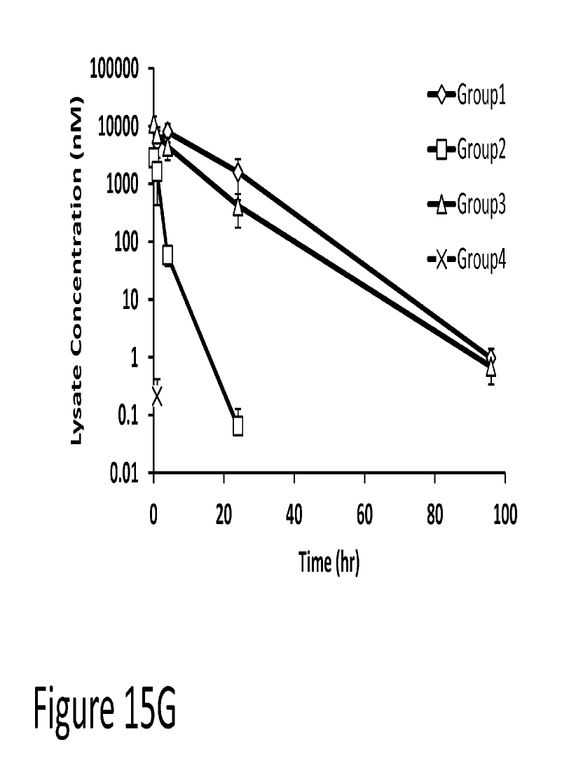

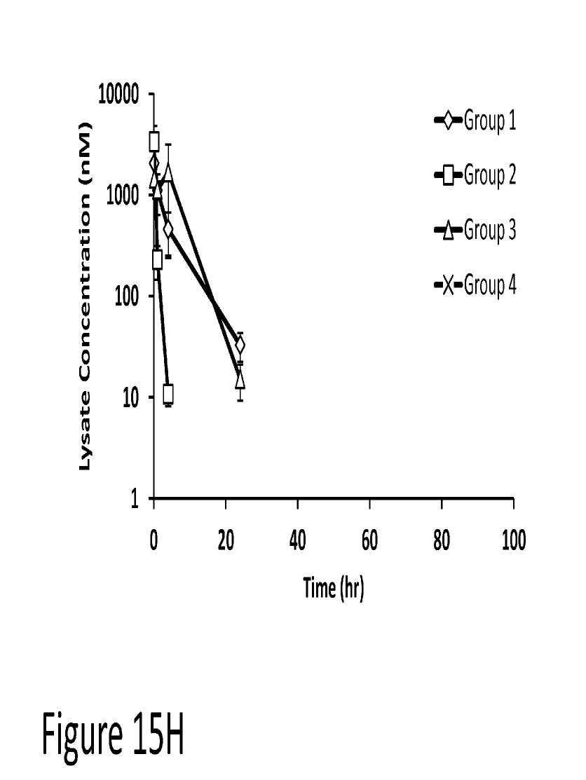

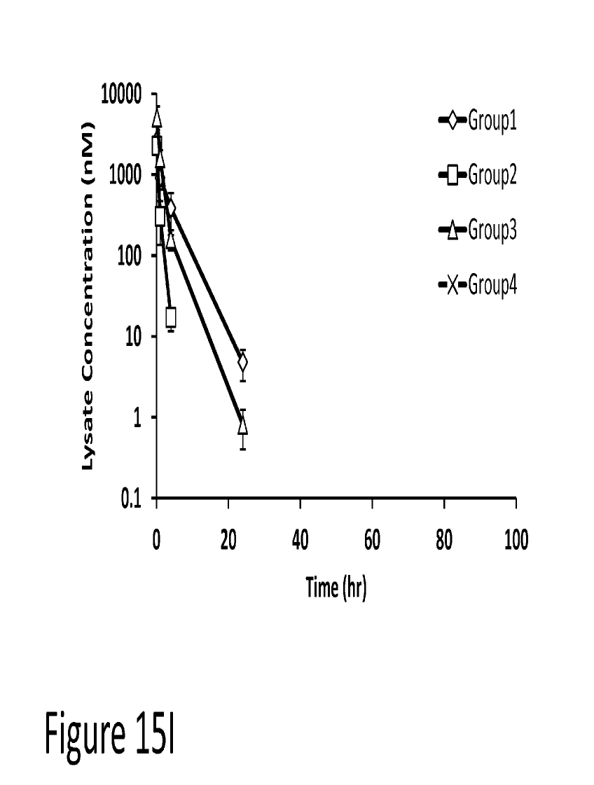

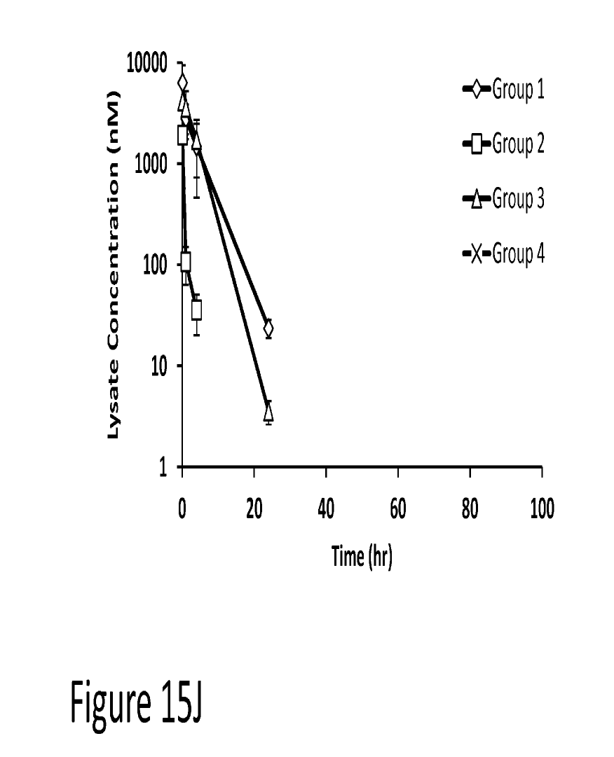

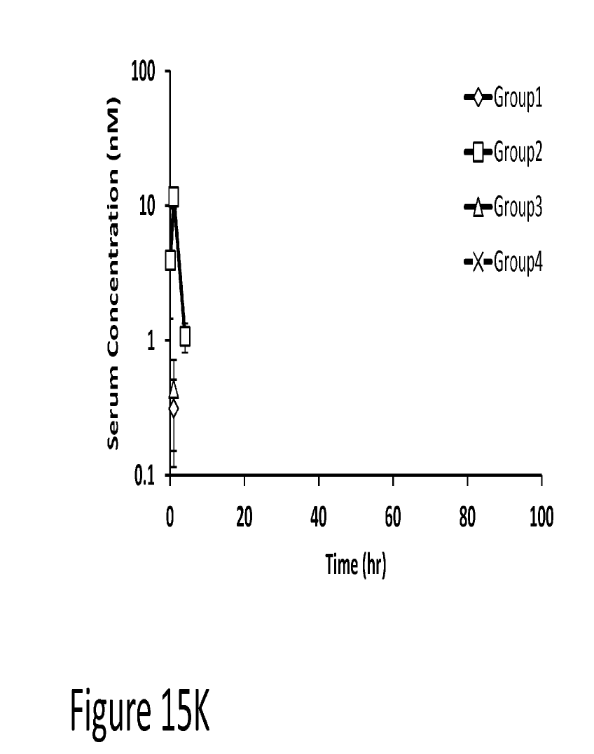

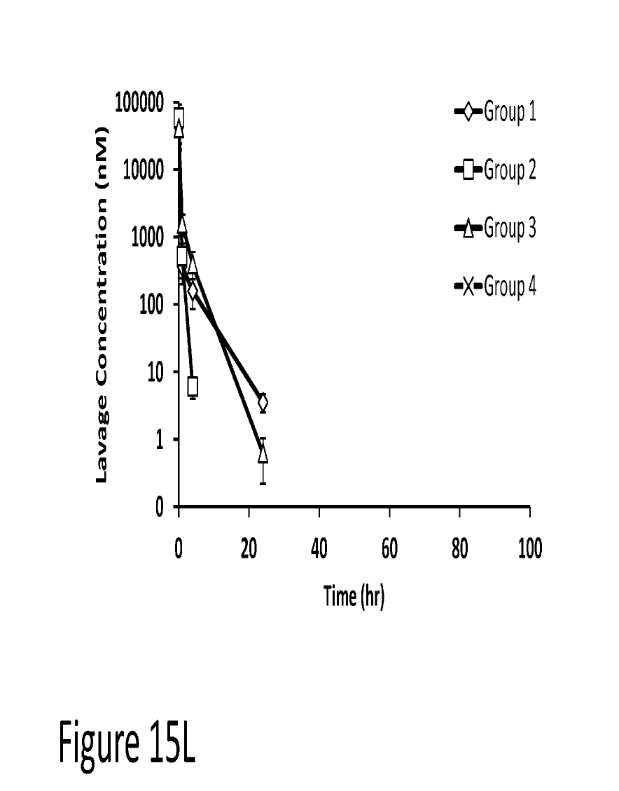

Dexamethasone retention for the Group 1 formulation was at least 10-fold lower at the immediate time point, but more than 10-fold higher than Group 3 at all subsequent time points for cartilage, meniscus, ligament and patella plus synovium lysates (FIGS. 15A-D, except for the 4 hour time point in meniscus, FIG. 15B). Group 2 was at least 10-fold higher than Group 3 at 1 hour and later time points in these tissues (except for the 4 hr time point in meniscus, FIG. 15B). Group 1 was also approximately 10-fold lower than Group 2 at the immediate time point, but the same or higher than Group 2 at all subsequent time points in these tissues (FIGS. 15A-D).

The Group 1 formulation was not detectable in the serum at the immediate time point and was approximately 10-fold lower than either Group 2 or Group 3 from 1-24 hours (FIG. 15E). In the synovial lavage, Group 1 was 10-fold higher than Group 2 at 1 hour and 4 hours and 1000-fold higher than Group 3 at 4 hours in the synovial lavage (FIG. 15F).

These results show increased retention of dexamethasone in cartilage, meniscus, ligament, and patella plus synovium when delivered by lipid particles in Groups 1 and 2 as compared to the immediate release injectable formulation in Group 3. In addition, the palmitate functionalized dexamethasone pro-drug in Group 1 (dexamethasone-21-palmitate) reduced the burst release at the immediate time point as compared to both unfunctionalized dexamethasone Group 2 particles (dexamethasone) and the Group 3 immediate release formulation (dexamethasone phosphate, Anti-Infl-3) as shown by the lower levels of Group 1 in cartilage, meniscus, ligament and patella plus synovium at the immediate time point and the undetectable or lower levels for Group 1 in the serum than for Groups 2 or 3. This lower burst likely contributed to the sustained retention of Group 1 dexamethasone levels in the synovial lavage at 1 hour and 4 hours.

IGF was detected in cartilage tissue at 24 and 96 hours only for Groups 1 and 3, while IGF was detected for Group 2 at 24 hours in only 1 of the 6 animals (FIG. 15G). At 1 hour and 4 hours in cartilage, Groups 1 and 3 were approximately 4- and 100-fold higher than Group 2, respectively. In meniscus, ligament, patella+synovium and synovial lavage, IGF was detected for Groups 1 and 3 at 24 hours, but Group 2 was not (FIGS. 15H-J and FIG. 15L). At 1 hour and 4 hours IGF levels for Groups 1 & 3 were 2-100-fold higher than for Group 2 in these tissues. Serum IGF levels were approximately 10-fold higher for Group 2 than for Groups 1 & 3 at the immediate time point with Group 2 levels remaining detectable at 1 hour and 4 hours, while Groups 1 & 3 were below the limit of detection (FIG. 15K). Similar IGF retention levels were observed for Groups 1 & 3 in all tissues at all time points.

These results show preferential retention of IGF within the knee joint of rats for Groups 1 & 3, which contain GF-Fus3, the cartilage-binding engineered IGF fusion protein, as compared to Group 2, which contains wild-type, nonbinding IGF. IGF was retained longer in cartilage for Groups 1 & 3 than in meniscus, ligament, and patella plus synovium, likely due to the dramatically higher GAG content of cartilage compared to the other tissues which preferentially retains the heparin binding domain in GF-Fus3. No differences were seen between Groups 1 & 3 showing that the presence of lipid particles does not affect the retention of GF-Fus3.

### Example 14

### Equivalent Cartilage Retention and Anabolic Stimulus for GF-Fus3 with and without a Purification Tae in an In Vitro Joint Disease Model Washout Experiment Using Explanted Bovine Cartilage

GF-Fus3 was prepared both with and without a 6×-Histidine tag (GF-Fus3-His and GF-Fus3, respectively). The bovine cartilage explants were prepared and cultured as described above in Example 5 and the fusion protein treatments were included in the medium as described in Table 13. All explants are from the same animal.

TABLE 13
Experimental Design for Example 14

| Treatment | Time point | | | | | # explants |
|---|---|---|---|---|---|---|
| Condition | (day) | IL1α | GF-Fus1 | GF-Fus3-His | GF-Fus3 | (n) |
| Healthy | 12 | − | − | − | − | 6 |
| Disease | 12 | + | − | − | − | 6 |
| GF-Fus1 4D | 12 | + | +4 days | − | − | 6 |
| GF-Fus1 12D | 12 | + | +12 days | − | − | 6 |
| GF-Fus3-His 4D | 12 | + | − | +4 days | − | 6 |
| GF-Fus3-His 12D | 12 | + | − | +12 days | − | 6 |
| GF-Fus3 4D | 12 | + | − | − | +4 days | 6 |
| GF-Fus3 12D | 12 | + | − | − | +12 days | 6 |

### Results

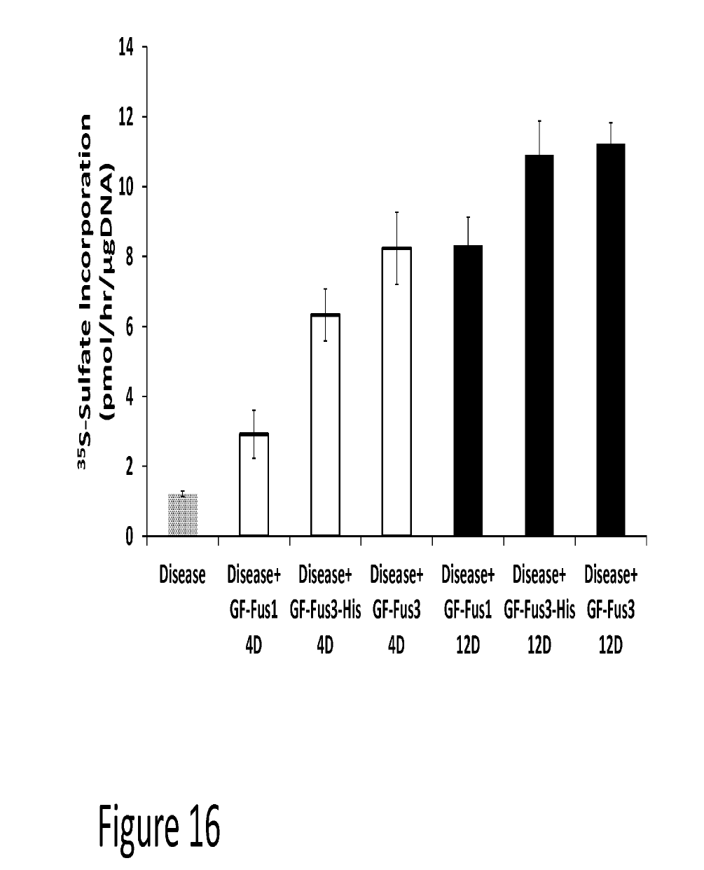

FIG. 16 shows that removal of the 6×-Histidine tag from GF-Fus3-His did not change the stimulation of cartilage matrix biosynthesis. In addition, consistent with previous examples, 12 days of continuous treatment with GF-Fus1, GF-Fus3-His, and GF-Fus3 stimulated cartilage matrix biosynthesis as compared to the Disease control. GF-Fus3-His and GF-Fus3 stimulated matrix biosynthesis as compared to the Disease control with 4 days of treatment more than 2-fold higher than the non-cartilage binding GF-Fus1.

### Example 15

### GF-Fus3 is Stable in Human Synovial Fluid from Donors with Minimal and Severe Cartilage Degeneration

Sustained treatment activity of damaged cartilage will require stability of cartilage targeted fusion proteins within the synovial cavity of a damaged joint. Therefore, the stability of GF-Fus3 with incubation in human synovial fluid from donors with cartilage degradation was determined

### Methods

Synovial fluid was harvested from two human donors (see Table 14) within 24 hours of death, flash frozen and stored at −80° C. 2 μL of GF-Fus3 (0.45 mg/mL in PBS, Life Technologies Cat#10010-031) was added to 18 μL of synovial fluid (final GF-Fus3 concentration is 45 μg/mL) in a sealed 200 μL PCR tube and incubated at 3TC for 0, 24, 48, 72, or 96 hours. Samples were diluted 1:10 in PBS and 10 μL (45 ng of GF-Fus3) was mixed with 3.3 μL of NuPAGE LDS Sample Buffer (Life Technologies, Cat# NP0007). GF-Fus3 standards were prepared at 100, 200, 400, 800, and 1600 ng/mL and 10 μL of each solution was mixed with 3.3 uL of NuPAGE LDS Sample Buffer. All samples and standards were loaded onto a 4-12% NuPAGE Bis-Tris Gel (Life Technologies, Cat# NP0321BOX), and run in NuPAGE MES SDS Running Buffer (Life Technologies, Cat# NP0002). Protein was transferred to a nitrocellulose membrane using an iBlot kit (Life Technologies, Cat# IB301001). Membrane was blocked in Odyssey Blocking Buffer (LI-COR, Cat#927-40000) for 1 hour at room temperature with agitation. Membrane was washed twice in PBS with 0.05% Tween20 (PBS-T) and incubated in 2 μg/mL anti-IGF-1 antibody (Millipore, Cat#05-172) overnight at 4° C. Membrane was washed three times in PBS-T and incubated with IRDye 800CW goat anti-mouse antibody (LI-COR, Cat#827-08364) diluted 1:2000 for 1 hour at room temperature. Membrane was washed three times with PBS-T and imaged on a LI-COR Odyssey CLx.

TABLE 14
Human Synovial Fluid Donors

| Age | Modified Collins Cartilage Grade | Sex |
|---|---|---|
| 63 | 1 | Female |
| 76 | 3 | Female |

### Results

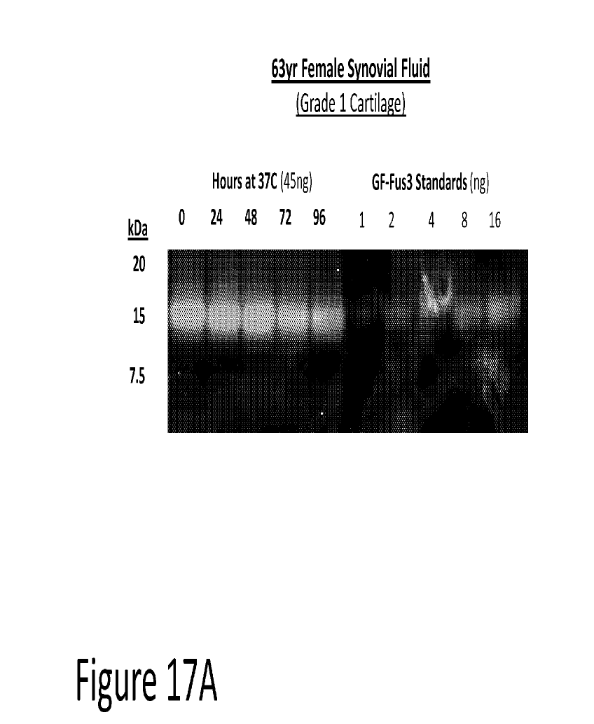

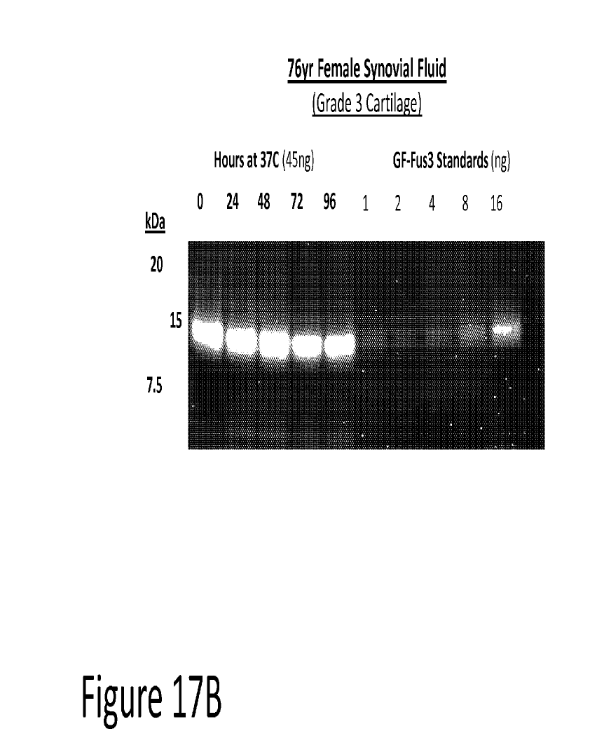

For donors with both grade 1 (minimal) and grade 3 (severe) cartilage degeneration (FIGS. 17A and 17B, respectively), 96 hours of 3TC incubation of GF-Fus3 in synovial fluid resulted in minimal protein degradation. When 45 μg of incubated protein was loaded, a faint band less than 7.5 kDa was visible in both FIGS. 17A and 17B, but the intensity of this band in both cases was less than or equivalent to the ing GF-Fus3 standard, suggesting 2% or less of the initial protein was degraded during synovial fluid incubation.

### EQUIVALENTS

Those skilled in the art will recognize, or be able to ascertain using no more than routine experimentation, many equivalents of the specific embodiments of the invention(s) described herein. Such equivalents are intended to be encompassed by the following claims. Any combination of one or more of the embodiments disclosed in any independent claim and any of the dependent claims is also contemplated to be within the scope of the invention.

### INCORPORATION BY REFERENCE

Each and every patent, pending patent application, and publication referred to herein is hereby incorporated herein by reference in its entirety.

1. A fusion protein comprising a first binding domain and a second binding domain, wherein, when present in the fusion protein, the first domain binds specifically to an extracellular domain of the growth factor receptor, and the second domain binds specifically to the cartilage matrix component.

2. The fusion protein of claim 1, wherein one or more of the following conditions are met:
a) the fusion protein is comprised of a single polypeptide chain;
b) the first binding domain is an IGF-1 receptor binding domain;
c) the second binding domain is a GAG (glycosaminoglycan) binding domain; and
d) the second binding domain is a collagen binding domain.

3-5. (canceled)

6. The fusion protein of claim 2, wherein the GAG binding domain comprises a sequence of, or a sequence homologous to, or substantially homologous to a GAG binding domain of proline-arginine-rich end leucine-rich repeat protein (PRELP), chondroadherin, oncostatin M, collagen IX, BMP-4, fibronectin, RAND1, RAND2, RAND3, RAND4, RAND5, RAND6, AKK15, RLR22, R1Q17, SEK20, ARK24, AKK24, ALI, AL2, AL3, LGT25, Pep184, Pep186, Pep185, Pep239, Pep246, ATIII, or FibB eta.

7. The fusion protein of claim 2, wherein one or more of the following conditions are met:
a) the GAG binding domain comprises an amino acid sequence selected from the group consisting of SEQ ID NOs:2-13, and 54-70;
b) the IGF-1 receptor binding domain comprises the amino acid sequence of human IGF-1; and
c) the collagen binding domain comprises a sequence of, or a sequence homologous to, or substantially homologous to the sequence of a collagen binding domain of CNA35, CNA344, thrombospondin, matrilin, cartilage oligomeric matrix protein, PRELP, cartilage oligomeric protein, chondroadherin, fibromodulin, decorin, or asporin.

8. The fusion protein of claim 2, wherein one or more of the following conditions are met:
a) the GAG binding domain comprises SEQ ID NO:2; and
b) the IGF-1 receptor binding domain comprises an amino acid sequence that comprises SEQ ID NO:1.

9-11. (canceled)

12. The fusion protein of claim 2, wherein the collagen binding domain comprises an amino acid sequence selected from the group consisting of SEQ ID NOs:14-16, and 21-27.

13. The fusion protein of claim 1, wherein one or more of the following conditions are met:
a) the fusion protein comprises an amino acid sequence selected from the group consisting of SEQ ID NOs:17-20, 28-53, and 71-87;
b) each binding domain, when present in the fusion protein, exhibits native binding activity; and
c) the fusion protein comprises fewer than 40,000, 35,000, 30,000, 25,000, 20,000, 15,000, 10,000, 7,500, 5,000, 2,500, 1,000, 500, or 250 amino acids.

14-15. (canceled)

16. The fusion protein of claim 1, wherein, upon injection into an intra-articular space of a joint of a mammal, the fusion protein is retained within cartilage tissue of the joint for a period of time that is at least: 1.5 times, 2 times, 3 times, four times, five times, six times, seven times, eight times, nine times, ten times, twenty times, forty times, fifty times, sixty times, seventy times, eighty times, ninety times, or one hundred times longer than a fusion mutein which differs from the fusion protein of claim 1 only in that the second binding domain is a mutant domain that does not specifically bind to the cartilage matrix component.

17. The fusion protein of claim 1, wherein one or more of the following conditions are met:
a) the joint is an injured joint or a diseased joint, and the amount of fusion protein retained in the cartilage tissue is at least about 5, about 10, about 20, or about 50 pmol/g of tissue;
b) the mammal is a rat or a horse, the joint is an injured joint or a diseased joint, and 8, 9, 10, 11, 12, 13, or 14 days following the injection, the joint exhibits a reduction in loss of 1) sGAG from the cartilage tissue, 2) cell content, 3) total cartilage tissue, or 4) bone quality, when compared to loss of 1), 2), 3) or 4) of a matched control joint that has been injected with a control protein;
c) the mammal is a rat or a horse, the joint is an injured joint or a diseased joint, and 8, 9, 10, 11, 12, 13, or 14 days following the injection the cartilage tissue is characterized by an increase in production of sGAG in the cartilage tissue, when compared to production of sGAG in cartilage tissue of a matched control joint that has been injected with a control protein; and
d) the mammal is a rat or a horse, the joint is an injured joint or a diseased joint, and 8, 9, 10, 11, 12, 13, or 14 days following the injection the cartilage tissue is characterized by an increase in levels of sGAG in the cartilage tissue, when compared to levels of sGAG in cartilage tissue of a matched control joint that has been injected with a control protein.

18-20. (canceled)

21. A composition comprising the fusion protein of claim 1, said composition further comprising a glucocorticoid, wherein optionally the glucocorticoid is selected from the group consisting of alclometasone, beclometasone, betamethasone, budesonide, chloroprednisone, ciclesonide, cortisol, cortisporin, cortivazol, deflazacort, dexamethasone, fludroxycortide, flunisolide, fluocinonide, fluocortolone, fluorometholone, fluticasone, hexacetonhydrocortamate, hydrocortisone, meprednisone, methylprednisolone, mometasone, paramethasone, prednisolone, prednisone, prednylidene, pregnadiene, pregnatriene, pregnene, proctosedyl, rimexolone, tetrahydrocorticosterone, triamcinolone and ulobetasol, and pharmaceutically acceptable salts, hydrates and esters thereof.

22. The composition of claim 21, wherein one or more of the following conditions are met:
a) the glucocorticoid is present at a concentration of 1-1000 μg/g of the composition;
b) the glucocorticoid is conjugated to a fatty acid and the conjugation to the fatty acid is optionally via an ester bond; and
c) the glucocorticoid is contained in a microparticle carrier.

23. (canceled)

24. The composition of claim 22, wherein one or more of the following conditions are met:
a) the fatty acid comprises palmitic acid;
b) the microparticle carrier is a liposome;
c) the microparticle carrier is a multilamellar vesicle;
d) the microparticle carrier comprises a high melting temperature lipid; and
e) the glucocorticoid is present in the microparticle carrier at a concentration of between 0.1-20 molar percent of the microparticle carrier lipid.

25-28. (canceled)

29. The composition of claim 24, wherein the lipid comprises distearoylphosphatidylcholine (DSPC), Dipalmitoylphosphatidylcholine (DPPC), or Hydro Soy phosphatidylcholine (HSPC).

30. (canceled)

31. A composition comprising a fusion protein having the amino acid sequence set forth in SEQ ID NO:18 and dexamethasone 21-palmitate, wherein the dexamethasone 21-palmitate is contained in an HSPC-containing multilamellar vesicle.

32. The composition of claim 21, wherein, after injection of the composition into an intra-articular space of an injured joint or a diseased joint, cartilage matrix synthesis readouts or cartilage degradation readouts are obtained, and the readouts show improvement over control readouts obtained after matched injection of a matched composition without glucocorticoid.

33. A method of treatment of a joint injury or disease, the method comprising administration into an intra-articular space of a joint a therapeutically effective amount of the fusion protein of claim 1.

34. The method of claim 33, wherein the joint injury or disease is selected from osteoarthritis, rheumatoid arthritis, cartilage degradation, acute inflammatory arthritis, infectious arthritis, osteoporosis, a drug toxicity-related cartilage defect, or a traumatic cartilage injury.

35. The composition of claim 31, wherein, after injection of the composition into an intra-articular space of an injured joint or a diseased joint, cartilage matrix synthesis readouts or cartilage degradation readouts are obtained, and the readouts show improvement over control readouts obtained after matched injection of a matched composition without glucocorticoid.

36. A method of treatment of a joint injury or disease, the method comprising administration into an intra-articular space of a joint a therapeutically effective amount of the composition of claim 21.

37. A method of treatment of a joint injury or disease, the method comprising administration into an intra-articular space of a joint a therapeutically effective amount of the composition of claim 31.
